(19) United States
(12) Patent Application Publication
(10) Pub. No.: US 2023/0081232 A1
(43) Pub. Date: Mar. 16, 2023

(54) SYSTEMS AND METHODS FOR MACHINE LEARNING FEATURES IN BIOLOGICAL SAMPLES

(71) Applicant: 10x Genomics, Inc., Pleasanton, CA (US)

(72) Inventors: Neil Ira Weisenfeld, Lynnfield, ME (US); Florian Baumgartner, Stockholm (SE); Alvaro J. Gonzalez Lozano, Livermore, CA (US)

(73) Assignee: 10x Genomics, Inc., Pleasanton, CA (US)

(21) Appl. No.: 17/799,198

(22) PCT Filed: Feb. 17, 2021
(86) PCT No.: PCT/US2021/018388

§ 371 Date: Aug. 11, 2022

Related U.S. Application Data
(60) Provisional application No. 62/977,565, filed on Feb. 17, 2020.

(51) Int. Cl. G06V 20/69; G06V 10/774; G06V 10/776; G16H 50/30

(52) U.S. Cl. CPC G06V 20/695; G06V 10/774; G06V 10/776; G16H 50/30

(57) ABSTRACT

Systems and methods for machine learning tissue classification are provided herein. Datasets for a plurality of biological samples are first generated. The dataset of each biological sample includes image data of the biological sample and molecular measurement data of the biological sample captured at a plurality of capture areas of the biological sample. The capture areas of the biological sample are registered to corresponding locations in the image data of the biological sample. Then, a machine learning module is trained with the datasets. Another dataset for another biological sample is generated (e.g., in the same or similar manner as the other datasets). And, the other dataset of the other biological sample is processed through the trained machine learning module to predict features in the other biological sample.

### CROSS REFERENCE TO RELATED APPLICATIONS

This patent application claims priority to, and thus the benefit of an earlier filing date from, U.S. Provisional Application No. 62/977,565, filed Feb. 17, 2020, the contents of which are hereby incorporated by reference.

### BACKGROUND

Resolution of analytes in complex tissues provides new insights into processes underlying biological function and morphology, such as cell fate and development, disease progression and detection, and cellular and tissue-level regulatory networks. Understanding patterns or other forms of relationships between analytes can provide information on differential cell behavior. This in turn, can help to elucidate complex conditions, such as complex diseases. For example, determining that the abundance of an analyte (e.g., a gene, a protein, etc.) is associated with a tissue subpopulation of a particular tissue class (e.g., disease tissue, healthy tissue, the boundary of disease and healthy tissue, etc.) can provide inferential evidence of the association of the analyte with a condition, such as complex disease. Similarly, determining that the abundance of an analyte is associated with a particular subpopulation of a heterogeneous cell population in a complex 2-dimensional or 3-dimensional tissue (e.g., a mammalian brain, liver, kidney, heart, an organoid, a tumor, a developing embryo of a model organism, or the like) can provide inferential evidence of the association of the analyte to a particular tissue subpopulation. Thus, analysis of analytes can provide information for the early detection of disease by identifying at-risk regions in complex tissues and characterizing the analyte profiles present in these regions.

### SUMMARY

The following presents a summary of the present disclosure in order to provide a basic understanding of some of the aspects of the present disclosure. This summary is not an extensive overview of the present disclosure. Rather, its purpose is to present some of the concepts of the present disclosure in a simplified form as a prelude to the more detailed description that is presented later.

With this in mind, certain technical solutions (e.g., computing systems, methods, and non-transitory computer readable storage mediums) for addressing tissue detection and binary tissue classification are provided in the present disclosure. In particular, the present disclosure provides systems and methods for the identification of and/or the prediction of analytes and other features in an image of a tissue section or slice via machine learning. Alternatively or additionally, the systems and methods presented herein may identify or otherwise predict images and/or disease states in the tissue section via machine learning.

In one embodiment, a dataset of a plurality of biological samples (e.g., sectioned tissue samples) is generated. Generally, this includes, for each biological sample, obtaining image data of the biological sample and molecular measurement data of the biological sample (e.g., one or more analytes of the biological sample) captured at a plurality of capture areas of the biological sample. In some embodiments, fiducial markers are used to align the molecular measure data of the biological sample with the image of the biological sample. In this regard, the capture areas of the biological sample are registered to corresponding locations in the image data of the biological sample. Then, a machine learning module is trained with the datasets. Another dataset for another biological sample is generated (e.g., in the same manner as the other datasets). And, the other dataset of the other biological sample is processed through the trained machine learning module to identify gene expression, protein expression, and/or other features in the other biological sample.

Another aspect of the present disclosure provides a computing system including one or more processors and memory storing one or more programs for tissue classification. The one or more programs are configured for execution by the one or more processors. The one or more programs include instructions for performing any of the methods disclosed above.

Still another aspect of the present disclosure provides a computer readable storage medium storing one or more programs to be executed by an electronic device. The one or more programs include instructions for the electronic device to perform binary tissue classification by any of the methods disclosed above.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary spatial analysis workflow.

FIG. 2 shows an exemplary spatial analysis workflow in which optional steps are indicated by dashed boxes.

FIG. 3 shows an exemplary spatial analysis workflow in which optional steps are indicated by dashed boxes.

FIG. 4 shows an exemplary spatial analysis workflow in which optional steps are indicated by dashed boxes.

FIG. 5 shows an exemplary spatial analysis workflow in which optional steps are indicated by dashed boxes.

FIG. 6 is a schematic diagram showing an example of a barcoded capture probe attached to a capture spot, as described herein.

FIG. 7 is a schematic illustrating a cleavable capture probe, in which the cleaved capture probe is configured to enter into a non-permeabilized cell and bind to target analytes within the sample.

FIG. 8 is a schematic diagram of an exemplary multiplexed spatially-labelled capture spot.

FIG. 9 is a schematic showing the arrangement of barcoded capture spots within an array.

FIG. 10 is a schematic illustrating a side view of a diffusion-resistant medium, e.g., a lid.

FIG. 11 is an example block diagram illustrating a computing device in accordance with some embodiments of the present disclosure.

FIGS. 12A-12F illustrate non-limiting methods for tissue classification in accordance with some embodiments of the present disclosure, in which optional steps are illustrated by dashed line boxes.

FIGS. 13A-13I illustrate the image input FIG. 13A of a tissue section overlayed on a substrate, the outputs of a variety of heuristic classifiers FIGS. 13B, 13C, 13D, 13E, 13F, 13G, and the outputs of a segmentation algorithm FIGS. 13H and 13I in accordance with some embodiments.

FIG. 14 is a block diagram of an exemplary system for machine learning features in a biological sample.

FIG. 15 is a block diagram illustrating an exemplary registration of image data to capture areas of a biological sample.

FIGS. 16-18 further illustrate the exemplary registration of image data to capture areas of a biological sample.

FIG. 19 is a flowchart of an exemplary registration process of the system of FIG. 14.

FIG. 20 is another block diagram of the system of FIG. 14 that may determine the accuracy of a machine learning module.

FIG. 21 is a block diagram of the system of FIG. 14 implemented in an exemplary cloud computing embodiment.

### DETAILED DESCRIPTION

### I. Introduction

This disclosure describes apparatus, systems, methods, and compositions for spatial analysis of biological samples. This section in particular describes certain general terminology, analytes, sample types, and preparative steps that are referred to in later sections of the disclosure.

Through spatial reconstruction (e.g., of gene expression, protein expression, DNA methylation, and/or single nucleotide polymorphisms, epigenetic perturbations, peptide localization, among others), a high-resolution spatial mapping of analytes to their specific location within a region or subregion reveals spatial expression of analytes, provides relational data, and further implicates analyte network interactions relating to disease or other morphologies or phenotypes of interest, resulting in a holistic understanding of cells in their morphological context.

Spatial analysis of analytes can be performed by capturing analytes and mapping them to known locations (e.g., using barcoded capture probes attached to a substrate) using a reference image indicating the tissues or regions of interest that correspond to the known locations. For example, in some implementations of spatial analysis, a sample is prepared (e.g., fresh-frozen tissue is sectioned, placed onto a preparation slide, fixed, and/or stained for imaging). Imaging of the sample provides the reference image to be used for spatial analysis. Molecular measurements may then be obtained using, e.g., analyte capture via barcoded capture probes, library construction, and/or sequencing. The resulting molecular measurement data and the reference image can be combined during data visualization for spatial analysis.

In addition to ensuring that a sample or an image of a sample (e.g., a tissue section or an image of a tissue section) is properly aligned with the capture techniques (e.g., using fiducial alignment), it may be necessary to determine which barcoded capture probes contain analyte data corresponding to the sample and which barcoded capture probes correspond to background. Thus, in order to map each analyte to a corresponding location in a sample or tissue (e.g., using the barcoded capture probes), it may be desirable to first be able to distinguish the regions of interest in the image (e.g., regions corresponding to sample and/or tissue) from regions that are not of interest (e.g., regions corresponding to background and/or non-tissue). Such a method reduces the amount of background signal noise during detection and spatial analysis of analytes, thus providing greater resolution when comparing analyte levels between regions of interest. For example, such a method can be used to compare analyte levels between a plurality of tissue subpopulations (e.g., mapping analyte profiles of disease tissue versus healthy tissue, such as a cancerous lesion in a tissue section) without the presence of confounding signals from background regions that minimize or distort true variations in the data (e.g., using normalization and/or reduction of high background signal to more distinctly reveal differential analyte levels in regions, to prevent low analyte signals from being discounted as background and/or to account for analyte diffusion away from the tissue on the substrate).

Technical limitations in the field are further compounded by the frequent introduction of imperfections in sample quality during conventional wet-lab methods for tissue sample preparation and sectioning. These issues arise either due to the nature of the tissue sample itself (including, inter alia, interstitial regions, vacuoles and/or general granularity that is often difficult to interpret after imaging) or from improper handling or sample degradation resulting in gaps or holes in the sample (e.g., tearing samples or obtaining only a partial sample such as from a biopsy). Additionally, wet-lab methods for imaging may result in further imperfections, including but not limited to air bubbles, debris, crystalline stain particles deposited on the substrate or tissue, inconsistent or poor-contrast staining, and/or microscopy limitations that produce image blur, over- or under-exposure, and/or poor resolution.

Conventional methods for image processing and identification of biological images typically require human input. For example, in order to identify a region of interest (e.g., a tissue section overlayed onto a substrate) using conventional tools (e.g., Magic Wand, Intelligent Scissors, Knockout 2, Graph Cut, among others), a practitioner may be required to select at least a part of a region that is desired (e.g., tissue) and/or undesired (e.g., non-tissue).

This necessity renders conventional tools less effective and substantially less robust because manual analysis by human eye requires a large amount of effort and time, is prone to human error and bias, and requires additional labor and cost of training in order for a practitioner to become skilled, e.g., in pathology or biological image analysis. Conventional methods for image analysis are also less efficient because the requirement for human input serves as a bottleneck that precludes rapid imaging, analyte capture and analysis during, for example, high-throughput applications where many tissue samples from multiple subjects must be processed at once. Such limitations severely reduce the power of high-throughput methods such as next-generation sequencing of nucleic acid analytes in tissue.

Therefore, there is a need in the art for a high-throughput, automated tissue classification systems and methods that can distinguish tissue from background in an image representative of a tissue overlayed onto a substrate (e.g., for analyte capture). Such systems and methods would allow reproducible identification of tissue samples in images without the need for extensive training and labor costs, and would further improve the accuracy of identification by removing human error due to subjective assessment. Such systems and methods would further provide a cost-effective, user-friendly tool for a practitioner to reliably perform spatial reconstruction of analytes in tissue sections without the need for additional user input during the spatial mapping step beyond providing the image.

(a) Spatial Analysis

Tissues and cells obtained from a mammal (e.g., a human) often have varied analyte levels (e.g., gene and/or protein expression) which can result in differences in cell morphology and/or function. The position of a cell within a tissue can affect, for example, cell fate, behavior, morphology, signaling and cross-talk with other cells in the tissue. Information regarding the differences in analyte levels within different cells in a tissue of a mammal can help physicians select or administer a treatment that will be effective in the mammal based on the detected differences in analyte levels within different cells in the tissue. Differences in analyte levels within different cells in a tissue of a mammal can also provide information on how tissues (e.g., healthy and diseased tissues) function and/or develop, on different mechanisms of disease pathogenesis in a tissue, or on the mechanism of action of a therapeutic treatment within a tissue. Furthermore, such differences in analyte levels can provide information on the mechanisms and development of drug resistance in mammalian tissues.

Spatial analysis methodologies herein provide for the detection of differences at the analyte level (e.g., gene and/or protein expression) within different cells in a tissue of a mammal or within a single cell from a mammal. For example, spatial analysis methodologies can be used to detect the differences in analyte levels within different cells in histological slide samples, the data from which can be reassembled to generate a three-dimensional map of the analyte levels of a tissue sample obtained from a mammal, with a degree of spatial resolution (e.g., single-cell resolution).

Spatial heterogeneity in developing systems has typically been studied using RNA hybridization, immunohistochemistry, fluorescent reporters, or purification or induction of pre-defined subpopulations and subsequent genomic profiling (e.g., RNA-seq). Such approaches, however, rely on a relatively small set of pre-defined markers, thus introducing selection bias that limits discovery. For example, spatial RNA assays traditionally rely on staining for a limited number of RNA species. In contrast, established methods for single-cell RNA-sequencing allows for broad, deep profiling of cellular gene expression but separate cells from their native spatial context.

Current spatial analysis methodologies provide a vast amount of data on analyte level and/or expression for a variety of multiple analytes within a sample at high spatial resolution, and, in some cases, while retaining the native spatial context. Such spatial analysis methods include, for example, the use of a capture probe including a spatial barcode (e.g., a nucleic acid sequence) that provides information as to the position of the capture probe within a cell or a tissue sample (e.g., a mammalian cell or a mammalian tissue sample) and a capture domain that is capable of binding to an analyte (e.g., a protein and/or nucleic acid) produced by and/or present in the cell. As described herein, a spatial barcode can be a nucleic acid that has a unique sequence, a unique fluorophore or a unique combination of fluorophores, or any other unique detectable agent. The capture domain can be any agent that is capable of binding to an analyte produced by and/or present in a cell (e.g., a nucleic acid that is capable of hybridizing to a nucleic acid from a cell (e.g., an mRNA, genomic DNA, mitochondrial DNA, or miRNA), a substrate or binding partner of an analyte, or an antibody that binds specifically to an analyte). A capture probe can also include a nucleic acid sequence that is complementary to a sequence of a universal forward and/or universal reverse primer. A capture probe can also include a cleavage site (e.g., a cleavage recognition site of a restriction endonuclease), or a photolabile or thermosensitive bond.

The binding of an analyte to a capture probe can be detected using a number of different methods, e.g., nucleic acid sequencing, fluorophore detection, nucleic acid amplification, detection of nucleic acid ligation, and/or detection of nucleic acid cleavage products. In some examples, the detection is used to associate a specific spatial barcode with a specific analyte produced by and/or present in a cell (e.g., a mammalian cell).

Capture probes can be, e.g., attached to a surface, e.g., a solid array, a bead, a flowcell, a wafer, or a coverslip. In some examples, capture probes are not attached to a surface.

In some examples, a cell or a tissue sample including a cell are contacted with capture probes attached to a substrate (e.g., a surface of a substrate), and the cell or tissue sample is permeabilized to allow analytes to be released from the cell and bind to the capture probes attached to the substrate. In some examples, analytes released from a cell can be actively directed to the capture probes attached to a substrate using a variety of methods, e.g., electrophoresis, chemical gradient, pressure gradient, fluid flow, or magnetic field.

In other examples, a capture probe is directed to interact with a cell or a tissue sample using a variety of methods, e.g., inclusion of a lipid anchoring agent in the capture probe or on the surface of the substrate, inclusion of an agent that binds specifically to, or forms a covalent bond with, a membrane protein.

(b) General Terminology

Specific terminology is used throughout this disclosure to explain various aspects of the apparatus, systems, methods, and compositions that are described. This sub-section includes explanations of certain terms that appear in later sections of the disclosure. To the extent that the descriptions in this section are in apparent conflict with usage in other sections of this disclosure, the definitions in this section will control.

(i) Subject

A “subject” is an animal, such as a mammal (e.g., human or a non-human simian), or avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum.

(ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or nucleic acid sequence, is intended as a semantic identifier for the nucleic acid or sequence in the context of a method or composition, and does not limit the structure or function of the nucleic acid or sequence beyond what is expressly indicated.

(iv) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

(v) Capture Spot

A “capture spot” (alternately, “feature” or “capture probe plurality”) is used herein to describe an entity that acts as a support or repository for various molecular entities used in sample analysis. Examples of capture spots include, but are not limited to, a bead, a spot of any two- or three-dimensional geometry (e.g., an ink jet spot, a masked spot, a square on a grid), a well, and a hydrogel pad. In some embodiments, a capture spot is an area on a substrate at which capture probes comprising spatial barcodes are clustered. Specific non-limiting embodiments of capture spots and substrates are further described below in the present disclosure.

(c) Analytes

The apparatus, systems, methods, and compositions described in this disclosure can be used to detect and analyze a wide variety of different analytes. For the purpose of this disclosure, an “analyte” can include any biological substance, structure, moiety, or component to be analyzed. The term “target” can be similarly used to refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins, lipoproteins, phosphoproteins, specific phosphorylated or acetylated variants of proteins, viral coat proteins, extracellular and intracellular proteins, antibodies, and antigen binding fragments. In some embodiments, the analyte can be an organelle (e.g., nuclei or mitochondria).

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, tagmented DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein).

Analytes can include a nucleic acid molecule with a nucleic acid sequence encoding at least a portion of a V(D)J sequence of an immune cell receptor (e.g., a TCR or BCR). In some embodiments, the nucleic acid molecule is cDNA first generated from reverse transcription of the corresponding mRNA, using a poly(T) containing primer. The generated cDNA can then be barcoded using a capture probe, featuring a barcode sequence (and optionally, a UMI sequence) that hybridizes with at least a portion of the generated cDNA. In some embodiments, a template switching oligonucleotide hybridizes to a poly(C) tail added to a 3′ end of the cDNA by a reverse transcriptase enzyme. The original mRNA template and template switching oligonucleotide can then be denatured from the cDNA and the barcoded capture probe can then hybridize with the cDNA and a complement of the cDNA generated. V(D)J analysis can also be completed with the use of one or more labelling agents that bind to particular surface features of immune cells and associated with barcode sequences. The one or more labelling agents can include an MHC or MHC multimer.

As described above, the analyte can include a nucleic acid capable of functioning as a component of a gene editing reaction, such as, for example, clustered regularly interspaced short palindromic repeats (CRISPR)-based gene editing. Accordingly, the capture probe can include a nucleic acid sequence that is complementary to the analyte (e.g., a sequence that can hybridize to the CRISPR RNA (crRNA), single guide RNA (sgRNA), or an adapter sequence engineered into a crRNA or sgRNA).

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

In general, the systems, apparatus, methods, and compositions can be used to analyze any number of the same or different analytes present in a region of the sample or within an individual capture spot of the substrate.

(d) Biological Samples

(i) Types of Biological Samples

A “biological sample” is obtained from the subject for analysis using any of a variety of techniques including, but not limited to, biopsy, surgery, and laser capture microscopy (LCM), and generally includes cells and/or other biological material from the subject. In addition to the subjects described above, a biological sample can also be obtained from a prokaryote such as a bacterium, e.g., Escherichia coli, Staphylococci or Mycoplasma pneumoniae; an archae; a virus such as Hepatitis C virus or human immunodeficiency virus; or a viroid. A biological sample can also be obtained from a eukaryote, such as a patient derived organoid (PDO) or patient derived xenograft (PDX). Subjects from which biological samples can be obtained can be healthy or asymptomatic individuals, individuals that have or are suspected of having a disease (e.g., a patient with a disease such as cancer) or a pre-disposition to a disease, and/or individuals that are in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient.

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

As discussed above, a biological sample can include a single analyte of interest, or more than one analyte of interest. Methods for performing multiplexed assays to analyze two or more different analytes in a single biological sample will be discussed in a subsequent section of this disclosure.

(ii) Preparation of Biological Samples

A variety of steps can be performed to prepare a biological sample for analysis. Except where indicated otherwise, the preparative steps described below can generally be combined in any manner to appropriately prepare a particular sample for analysis.

(1) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of the maximum cross-sectional dimension of a cell. However, tissue sections having a thickness that is larger than the maximum cross-section cell dimension can also be used. For example, cryostat sections can be used, which can be, e.g., 10-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1 micrometers. Thicker sections can also be used if desired or convenient, e.g., at least 70, micrometers or more. Typically, the thickness of a tissue section is between 1-100 micrometers, but sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

(2) Freezing

In some embodiments, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C.

(3) Formalin Fixation and Paraffin Embedding

In some embodiments, the biological sample can be prepared using formalin-fixation and paraffin-embedding (FFPE), which are established methods. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by rinsing (e.g., 99.5% ethanol for 2 minutes, 96% ethanol for 2 minutes, and 70% ethanol for 2 minutes).

(4) Fixation

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA), and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

(5) Embedding

As an alternative to paraffin embedding described above, a biological sample can be embedded in any of a variety of other embedding materials to provide structural substrate to the sample prior to sectioning and other handling steps. In general, the embedding material is removed prior to analysis of tissue sections obtained from the sample. Suitable embedding materials include, but are not limited to, waxes, resins (e.g., methacrylate resins), epoxies, hydrogels, and agar.

(6) Staining

To facilitate visualization, biological samples can be stained using a wide variety of stains and staining techniques. In some embodiments, for example, a sample can be stained using any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetraoxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

(7) Hydrogel Embedding

In some embodiments, the biological sample can be embedded in a hydrogel matrix. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation, etc.). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

(8) Isometric Expansion

In some embodiments, a biological sample embedded in a hydrogel can be isometrically expanded. Isometric expansion methods that can be used include hydration, a preparative step in expansion microscopy, as described in Chen et al., Science 347(6221):543-548, 2015. Other suitable expansion methods for analysis of proteins and RNA include those set forth by Asano et al., Curr Protoc Cell Bio 80(1): e56, 2018).

Isometric expansion can be performed by anchoring one or more components of a biological sample to a gel, followed by gel formation, proteolysis, and swelling. Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with capture probes, as will be discussed in greater detail in a subsequent section.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and LabelX (see, Chen et al., Science 347(6221):543-548, 2015).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least two times its non-expanded size. In some embodiments, the sample is isometrically expanded to at least 2× and less than 20× of its non-expanded size.

(9) Substrate Attachment

In some embodiments, the biological sample can be attached to a substrate. Examples of substrates suitable for this purpose are described in detail below. Attachment of the biological sample can be irreversible or reversible, depending upon the nature of the sample and subsequent steps in the analytical method.

In certain embodiments, the sample can be attached to the substrate reversibly by applying a suitable polymer coating to the substrate, and contacting the sample to the polymer coating. The sample can then be detached from the substrate using an organic solvent that at least partially dissolves the polymer coating. Hydrogels are examples of polymers that are suitable for this purpose.

(10) Disaggregation of Cells

In some embodiments, the biological sample corresponds to cells (e.g., derived from a cell culture or a tissue sample). In a cell sample with a plurality of cells, individual cells can be naturally unaggregated. For example, the cells can be derived from a suspension of cells and/or disassociated or disaggregated cells from a tissue or tissue section.

Alternatively, the cells in the sample may be aggregated, and may be disaggregated into individual cells using, for example, enzymatic or mechanical techniques. Examples of enzymes used in enzymatic disaggregation include, but are not limited to, dispase, collagenase, trypsin, and combinations thereof. Mechanical disaggregation can be performed, for example, using a tissue homogenizer.

(11) Suspended and Adherent Cells

In some embodiments, the biological sample can be derived from a cell culture grown in vitro. Samples derived from a cell culture can include one or more suspension cells which are anchorage-independent within the cell culture. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M.

Samples derived from a cell culture can include one or more adherent cells which grow on the surface of the vessel that contains the culture medium.

(12) Tissue Permeabilization

In some embodiments, a biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Any suitable method for sample permeabilization can generally be used in connection with the samples described herein.

In some embodiments, where a diffusion-resistant medium is used to limit migration of analytes or other species during the analytical procedure, the diffusion-resistant medium can include at least one permeabilization reagent. For example, the diffusion-resistant medium can include wells (e.g., micro-, nano-, or picowells) containing a permeabilization buffer or reagents. In some embodiments, where the diffusion-resistant medium is a hydrogel, the hydrogel can include a permeabilization buffer. In some embodiments, the hydrogel is soaked in permeabilization buffer prior to contacting the hydrogel with a sample. In some embodiments, the hydrogel or other diffusion-resistant medium can contain dried reagents or monomers to deliver permeabilization reagents when the diffusion-resistant medium is applied to a biological sample. In some embodiments, the diffusion-resistant medium, (i.e. hydrogel) is covalently attached to a solid substrate (e.g., an acrylated glass slide). In some embodiments, the hydrogel can be modified to both contain capture probes and deliver permeabilization reagents. For example, a hydrogel film can be modified to include spatially-barcoded capture probes. The spatially-barcoded hydrogel film is then soaked in permeabilization buffer before contacting the spatially-barcoded hydrogel film to the sample. The spatially-barcoded hydrogel film thus delivers permeabilization reagents to a sample surface in contact with the spatially-barcoded hydrogel, enhancing analyte migration and capture. In some embodiments, the spatially-barcoded hydrogel is applied to a sample and placed in a permeabilization bulk solution. In some embodiments, the hydrogel film soaked in permeabilization reagents is sandwiched between a sample and a spatially-barcoded array. In some embodiments, target analytes are able to diffuse through the permeabilizing reagent soaked hydrogel and hybridize or bind the capture probes on the other side of the hydrogel. In some embodiments, the thickness of the hydrogel is proportional to the resolution loss. In some embodiments, wells (e.g., micro-, nano-, or picowells) can contain spatially-barcoded capture probes and permeabilization reagents and/or buffer. In some embodiments, spatially-barcoded capture probes and permeabilization reagents are held between spacers. In some embodiments, the sample is punch, cut, or transferred into the well, where a target analyte diffuses through the permeabilization reagent/buffer and to the spatially-barcoded capture probes. In some embodiments, resolution loss may be proportional to gap thickness (e.g., the amount of permeabilization buffer between the sample and the capture probes).

In some embodiments, permeabilization solution can be delivered to a sample through a porous membrane. In some embodiments, a porous membrane is used to limit diffusive analyte losses, while allowing permeabilization reagents to reach a sample. Membrane chemistry and pore size can be manipulated to minimize analyte loss. In some embodiments, the porous membrane may be made of glass, silicon, paper, hydrogel, polymer monoliths, or other material. In some embodiments, the material may be naturally porous. In some embodiments, the material may have pores or wells etched into solid material. In some embodiments, the permeabilization reagents are flowed through a microfluidic chamber or channel over the porous membrane. In some embodiments, the flow controls the sample's access to the permeabilization reagents. In some embodiments, a porous membrane is sandwiched between a spatially-barcoded array and the sample, where permeabilization solution is applied over the porous membrane. The permeabilization reagents diffuse through the pores of the membrane and into the tissue.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS).

(12) Selective Enrichment of RNA Species

In some embodiments, where RNA is the analyte, one or more RNA species of interest can be selectively enriched. For example, one or more species of RNA of interest can be selected by addition of one or more oligonucleotides to the sample. In some embodiments, the additional oligonucleotide is a sequence used for priming a reaction by a polymerase. For example, one or more primer sequences with sequence complementarity to one or more RNAs of interest can be used to amplify the one or more RNAs of interest, thereby selectively enriching these RNAs. In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Subsequent application of capture probes to the sample can result in improved capture of other species of RNA due to the reduction in non-specific RNA (rRNA) present in the sample.

(13) Other Reagents

Additional reagents can be added to a biological sample to perform various functions prior to analysis of the sample. In some embodiments, DNase and RNase inactivating agents or inhibitors such as proteinase K, and/or chelating agents such as EDTA, can be added to the sample.

In some embodiments, the sample can be treated with one or more enzymes. For example, one or more endonucleases to fragment DNA, DNA polymerase enzymes, and dNTPs used to amplify nucleic acids can be added. Other enzymes that can also be added to the sample include, but are not limited to, polymerase, transposase, ligase, DNAse, and RNAse.

In some embodiments, reverse transcriptase enzymes can be added to the sample, including enzymes with terminal transferase activity, primers, and template switch oligonucleotides. Template switching can be used to increase the length of a cDNA, e.g., by appending a predefined nucleic acid sequence to the termini of the cDNA.

(14) Pre-processing for Capture Probe Interaction

In some embodiments, analytes in a biological sample can be pre-processed prior to interaction with a capture probe. For example, prior to interaction with capture probes, polymerization reactions catalyzed by a polymerase (e.g., DNA polymerase or reverse transcriptase) are performed in the biological sample. In some embodiments, a primer for the polymerization reaction includes a functional group that enhances hybridization with the capture probe. The capture probes can include appropriate capture domains to capture biological analytes of interest (e.g., poly-dT sequence to capture poly(A) mRNA).

In some embodiments, biological analytes are pre-processed for library generation via next generation sequencing. For example, analytes can be pre-processed by addition of a modification (e.g., ligation of sequences that allow interaction with capture probes). In some embodiments, analytes (e.g., DNA or RNA) are fragmented using fragmentation techniques (e.g., using transposases and/or fragmentation buffers).

Fragmentation can be followed by a modification of the analyte. For example, a modification can be the addition through ligation of an adapter sequence that allows hybridization with the capture probe. In some embodiments, where the analyte of interest is RNA, poly(A) tailing can be performed. Addition of a poly(A) tail to RNA that does not naturally contain a poly(A) tail (e.g., non-polyadenalyted RNA species) can facilitate hybridization with a capture probe that includes a capture domain with a functional amount of poly(dT) sequence.

In some embodiments, prior to interaction with capture probes, ligation reactions catalyzed by a ligase are performed in the biological sample. In some embodiments, the capture domain includes a DNA sequence that has complementarity to a RNA molecule, where the RNA molecule has complementarity to a second DNA sequence, and where the RNA-DNA sequence complementarity is used to ligate the second DNA sequence to the DNA sequence in the capture domain. In these embodiments, direct detection of RNA molecules is possible.

In some embodiments, prior to interaction with capture probes, target-specific reactions are performed in the biological sample. Examples of target specific reactions include, but are not limited to, ligation of target specific adaptors, probes and/or other oligonucleotides, target specific amplification using primers specific to one or more analytes, and target-specific detection using in situ hybridization, DNA microscopy, and/or antibody detection. In some embodiments, a capture probe includes capture domains targeted to target-specific products (e.g., amplification or ligation).

### II. General Spatial Array-Based Analytical Methodology

This section of the disclosure describes methods, apparatus, systems, and compositions for spatial array-based analysis of biological samples.

(a) Spatial Analysis Methods

Array-based spatial analysis methods generally involve the transfer of one or more analytes from a biological sample to an array of capture spots on a substrate, each of which is associated with a unique spatial location on the array. Subsequent analysis of the transferred analytes includes determining the identity of the analytes and the spatial location of each analyte within the sample. The spatial location of each analyte within the sample is determined based on the capture spot to which each analyte is bound in the array, and the capture spot's relative spatial location within the array.

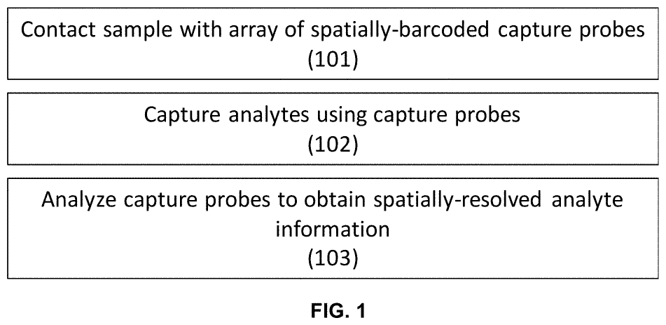

There are at least two general methods to associate a spatial barcode with one or more neighboring cells, such that the spatial barcode identifies the one or more cells, and/or contents of the one or more cells, as associated with a particular spatial location. One general method is to drive target analytes out of a cell and towards the spatially-barcoded array. FIG. 1 depicts an exemplary embodiment of this general method. In FIG. 1, the spatially-barcoded array populated with capture probes (as described further herein) is contacted with a sample 101, and sample is permeabilized 102, allowing the target analyte to migrate away from the sample and toward the array 102. The target analyte interacts with a capture probe on the spatially-barcoded array. Once the target analyte hybridizes/is bound to the capture probe, the sample is optionally removed from the array and the capture probes are analyzed in order to obtain spatially-resolved analyte information 103.

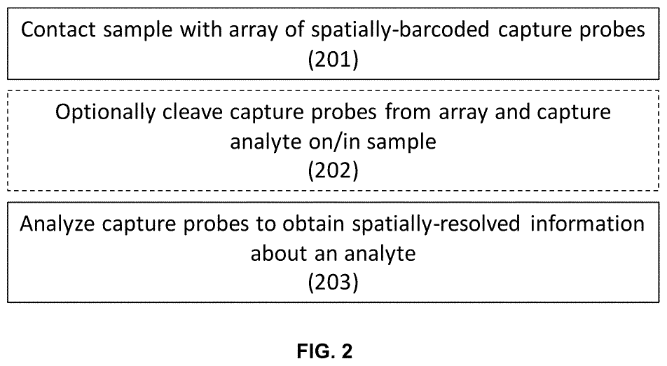

Another general method is to cleave the spatially-barcoded capture probes from an array, and drive the spatially-barcoded capture probes towards and/or into or onto the sample. FIG. 2 depicts an exemplary embodiment of this general method, the spatially-barcoded array populated with capture probes (as described further herein) can be contacted with a sample 201. The spatially-barcoded capture probes are cleaved and then interact with cells within the provided sample 202. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed to obtain spatially-resolved information about the tagged cell 203.

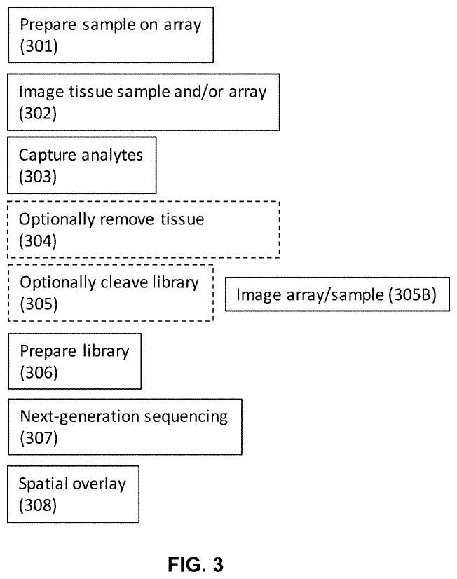

FIG. 3 shows an exemplary workflow that includes preparing a sample on a capture array 301. Sample preparation may include placing the sample on a slide, fixing the sample, and/or staining the sample for imaging. The stained sample is then imaged on the array 302 using both brightfield (to image the sample hematoxylin and eosin stain) and fluorescence (to image capture spots) modalities. In some embodiments, target analytes are then released from the sample and capture probes forming the spatial capture array hybridize or bind the released target analytes 303. The sample can be optionally removed from the array 304 and the capture probes can be optionally cleaved from the array 305. The sample and array are then imaged a second time in both modalities 305B while the analytes are reverse transcribed into cDNA, and an amplicon library is prepared 306 and sequenced 307. The two sets of images are then spatially-overlaid in order to correlate spatially-identified sample information 308.

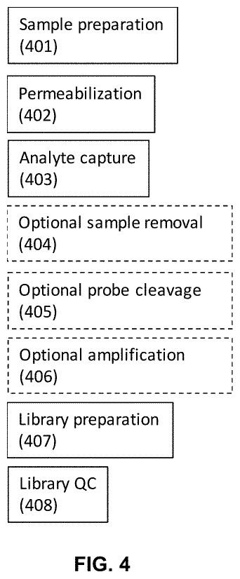

FIG. 4 shows another exemplary workflow that utilizes a spatially-labelled array on a substrate, where capture probes (e.g., labelled with spatial barcodes) are clustered at areas called capture spots. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatial capture array is contacted with a sample 401, and the sample is permeabilized through application of permeabilization reagents 402. Permeabilization reagents may be administered by placing the array/sample assembly within a bulk solution. Alternatively, permeabilization reagents may be administered to the sample via a diffusion-resistant medium and/or a physical barrier such as a lid, where the sample is sandwiched between the diffusion-resistant medium and/or barrier and the array-containing substrate. The analytes migrate toward the spatial capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatial capture probes, the capture probes can hybridize or otherwise bind a target analyte 403. The sample can be optionally removed from the array 404.

The capture probes can be optionally cleaved from the array 405, and the captured analytes can be spatially-tagged by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme. The original mRNA template and template switching oligonucleotide can then be denatured from the cDNA and the capture probe can then hybridize with the cDNA and a complement of the cDNA can be generated. The first stand cDNA can then be purified and collected for downstream amplification steps. The first strand cDNA can be optionally amplified using PCR 406, where the forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information 407, with an additional library quality control (QC) step 408.

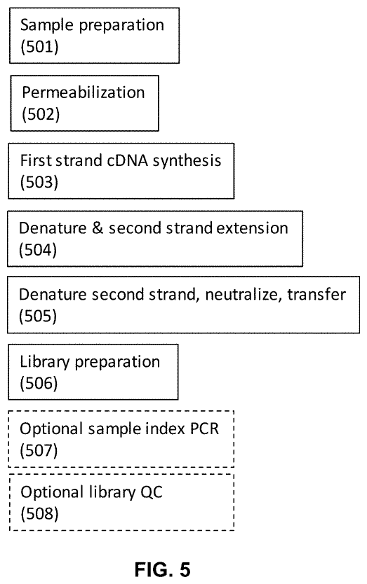

FIG. 5 depicts an exemplary workflow where the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. In this embodiment, sample preparation 501 and permeabilization 502 are performed as described elsewhere herein. Once the capture probes capture the target analyte(s), first strand cDNA created by template switching and reverse transcriptase 503 is then denatured and the second strand is then extended 504. The second strand cDNA is then denatured from the first strand cDNA, neutralized, and transferred to a tube 505. cDNA quantification and amplification can be performed using standard techniques discussed herein. The cDNA can then be subjected to library preparation 506 and optional indexing 507, including fragmentation, end-repair, and a-tailing, and indexing PCR steps. The library can also be optionally tested for quality control (QC) 508.

(b) Capture Probes

A “capture probe,” also interchangeably referred to herein as a “probe,” refers to any molecule capable of capturing (directly or indirectly) and/or labelling an analyte of interest in a biological sample. In some embodiments, the capture probe is a nucleic acid or a polypeptide. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

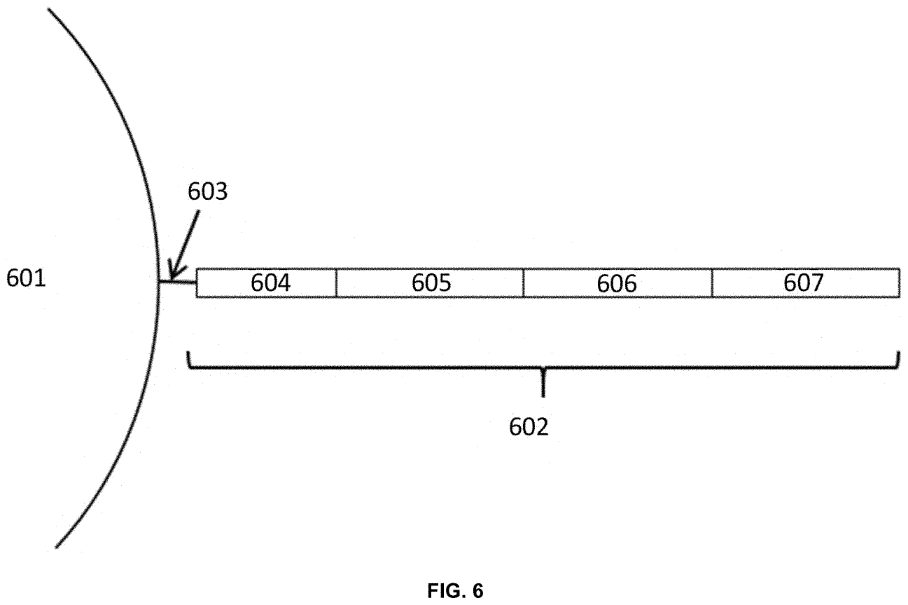

FIG. 6 is a schematic diagram showing one example of a capture probe. As shown, the capture probe 602 is optionally coupled to a capture spot 601 by a cleavage domain 603, such as a disulfide linker. The capture probe can include functional sequences that are useful for subsequent processing, such as functional sequence 604, which can include a sequencer specific flow cell attachment sequence, e.g., a P5 sequence, as well as functional sequence 606, which can include sequencing primer sequences, e.g., a R1 primer binding site. In some embodiments, sequence 604 is a P7 sequence and sequence 606 is a R2 primer binding site. A spatial barcode 605 can be included within the capture probe for use in barcoding the target analyte. The functional sequences can be selected for compatibility with a variety of different sequencing systems, e.g., 454 Sequencing, Ion Torrent Proton or PGM, Illumina X10, etc., and the requirements thereof. In some embodiments, the spatial barcode 605, functional sequences 604 (e.g., flow cell attachment sequence) and 606 (e.g., sequencing primer sequences) can be common to all of the probes attached to a given capture spot. The spatial barcode can also include a capture domain 607 to facilitate capture of a target analyte.

(i) Capture Domain

As discussed above, each capture probe includes at least one capture domain. The “capture domain” is an oligonucleotide, a polypeptide, a small molecule, or any combination thereof, that binds specifically to a desired analyte. In some embodiments, a capture domain can be used to capture or detect a desired analyte.

In some embodiments, the capture domain is a functional nucleic acid sequence configured to interact with one or more analytes, such as one or more different types of nucleic acids (e.g., RNA molecules and DNA molecules). In some embodiments, the functional nucleic acid sequence can include an N-mer sequence (e.g., a random or degenerate N-mer sequence), which N-mer sequences are configured to interact with a plurality of DNA molecules. In some embodiments, the functional sequence can include a poly(T) sequence, which poly(T) sequences are configured to interact with messenger RNA (mRNA) molecules via the poly(A) tail of an mRNA transcript. In some embodiments, the functional nucleic acid sequence is the binding target of a protein (e.g., a transcription factor, a DNA binding protein, or a RNA binding protein), where the analyte of interest is a protein.

Capture probes can include ribonucleotides and/or deoxyribonucleotides as well as synthetic nucleotide and nucleoside residues that are capable of participating in Watson-Crick type or analogous base pair interactions (e.g., inosine). In some embodiments, the capture domain is capable of priming a reverse transcription reaction to generate cDNA that is complementary to the captured RNA molecules. In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, SplintR ligase along with RNA or DNA sequences (e.g., degenerate RNA) can be used to ligate a single stranded DNA to the capture domain. In some embodiments, a capture domain includes a nucleotide sequence that is complementary to a splint oligonucleotide.

In some embodiments, the capture domain is located at the 3′ end of the capture probe and includes a free 3′ end that can be extended, e.g., by template dependent polymerization, to form an extended capture probe as described herein. In some embodiments, the capture domain includes a nucleotide sequence that is capable of hybridizing to nucleic acid, e.g., RNA or other analyte, present in the cells of the tissue sample contacted with the array. In some embodiments, the capture domain can be selected or designed to bind selectively or specifically to a target nucleic acid. For example, the capture domain can be selected or designed to capture mRNA by way of hybridization to the mRNA poly(A) tail. Thus, in some embodiments, the capture domain includes a poly(T) DNA oligonucleotide, i.e., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly-U oligonucleotide or an oligonucleotide included of deoxythymidine analogues. In some embodiments, the capture domain can include 10 or more nucleotides.

In some embodiments, random or degenerate sequences, e.g., random hexamers, random nonamers or similar sequences, can be used to form all or a part of the capture domain. For example, random or degenerate sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes poly(T)-like sequence and the capture domain of one or more capture probes includes random sequences. In some embodiments, a pool of two or more capture probes form a mixture where the capture domain of one or more capture probes includes a poly(T)-random sequences and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a particular gene sequence or particular motif sequence or common/conserved sequence, that it is designed to capture (i.e., a sequence-specific capture domain). Thus, in some embodiments, the capture domain is capable of binding selectively to a desired sub-type or subset of nucleic acid, for example a particular type of RNA, such as mRNA, rRNA, tRNA, SRP RNA, tmRNA, snRNA, snoRNA, SmY RNA, scaRNA, gRNA, RNase P, RNase MRP, TERC, SL RNA, aRNA, cis-NAT, crRNA, lncRNA, miRNA, piRNA, siRNA, shRNA, tasiRNA, rasiRNA, 7SK, eRNA, ncRNA or other types of RNA. In a non-limiting example, the capture domain can be capable of binding selectively to a desired subset of ribonucleic acids, for example, microbiome RNA, such as 16S rRNA.

In some embodiments, a capture domain includes an “anchor” or “anchoring sequence”, which is a sequence of nucleotides that is designed to ensure that the capture domain hybridizes to the intended biological analyte. In some embodiments, an anchor sequence includes a sequence of nucleotides, including a 1-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. Alternatively, the sequence can be designed using a specific sequence of nucleotides. In some embodiments, the anchor sequence is at the 3′ end of the capture domain. In some embodiments, the anchor sequence is at the 5′ end of the capture domain.

In some embodiments, capture domains of capture probes are blocked prior to contacting the biological sample with the array, and blocking probes are used when the nucleic acid in the biological sample is modified prior to its capture on the array. In some embodiments, the blocking probe is used to block or modify the free 3′ end of the capture domain. In some embodiments, blocking probes can be hybridized to the capture probes to mask the free 3′ end of the capture domain, e.g., hairpin probes or partially double stranded probes. In some embodiments, the free 3′ end of the capture domain can be blocked by chemical modification, e.g., addition of an azidomethyl group as a chemically reversible capping moiety such that the capture probes do not include a free 3′ end. Blocking or modifying the capture probes, particularly at the free 3′ end of the capture domain, prior to contacting the biological sample with the array, prevents modification of the capture probes, e.g., prevents the addition of a poly(A) tail to the free 3′ end of the capture probes.

Non-limiting examples of 3′ modifications include dideoxy C-3′ (3′-ddC), 3′ inverted dT, 3′ C3 spacer, 3′Amino, and 3′ phosphorylation. In some embodiments, the nucleic acid in the biological sample can be modified such that it can be captured by the capture domain. For example, an adaptor sequence (including a binding domain capable of binding to the capture domain of the capture probe) can be added to the end of the nucleic acid, e.g., fragmented genomic DNA. In some embodiments, this is achieved by ligation of the adaptor sequence or extension of the nucleic acid. In some embodiments, an enzyme is used to incorporate additional nucleotides at the end of the nucleic acid sequence, e.g., a poly(A) tail. In some embodiments, the capture probes can be reversibly masked or modified such that the capture domain of the capture probe does not include a free 3′ end. In some embodiments, the 3′ end is removed, modified, or made inaccessible so that the capture domain is not susceptible to the process used to modify the nucleic acid of the biological sample, e.g., ligation or extension.

In some embodiments, the capture domain of the capture probe is modified to allow the removal of any modifications of the capture probe that occur during modification of the nucleic acid molecules of the biological sample. In some embodiments, the capture probes can include an additional sequence downstream of the capture domain, i.e., 3′ to the capture domain, namely a blocking domain.

(ii) Cleavage Domain

Each capture probe can optionally include at least one cleavage domain. The cleavage domain represents the portion of the probe that is used to reversibly attach the probe to an array capture spot, as will be described further below. Further, one or more segments or regions of the capture probe can optionally be released from the array capture spot by cleavage of the cleavage domain. As an example spatial barcodes and/or universal molecular identifiers (UMIs) can be released by cleavage of the cleavage domain.

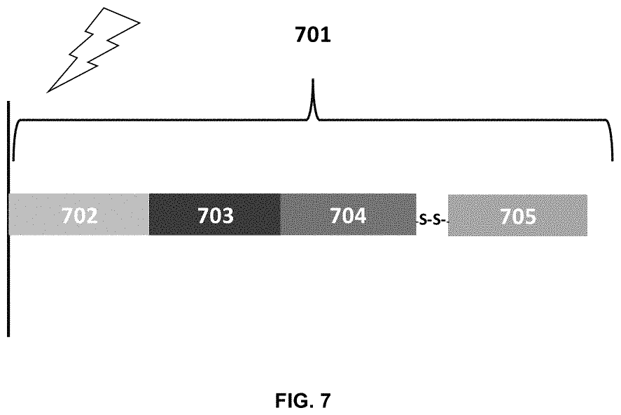

FIG. 7 is a schematic illustrating a cleavable capture probe, where the cleaved capture probe can enter into a non-permeabilized cell and bind to target analytes within the sample. The capture probe 701 contains a cleavage domain 702, a cell penetrating peptide 703, a reporter molecule 704, and a disulfide bond (—S—S—). 705 represents all other parts of a capture probe, for example a spatial barcode and a capture domain.

In some embodiments, the cleavage domain is a propylene residue (e.g., Spacer C3). In some embodiments, the cleavage domain linking the capture probe to a capture spot is a disulfide bond. A reducing agent can be added to break the disulfide bonds, resulting in release of the capture probe from the capture spot. As another example, heating can also result in degradation of the cleavage domain and release of the attached capture probe from the array capture spot. In some embodiments, laser radiation is used to heat and degrade cleavage domains of capture probes at specific locations. In some embodiments, the cleavage domain is a photo-sensitive chemical bond (i.e., a chemical bond that dissociates when exposed to light such as ultraviolet light).

Other examples of cleavage domains include labile chemical bonds such as, but not limited to, ester linkages (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

In some embodiments, the cleavage domain includes a sequence that is recognized by one or more enzymes capable of cleaving a nucleic acid molecule, e.g., capable of breaking the phosphodiester linkage between two or more nucleotides. A bond can be cleavable via other nucleic acid molecule targeting enzymes, such as restriction enzymes (e.g., restriction endonucleases). For example, the cleavage domain can include a restriction endonuclease (restriction enzyme) recognition sequence. Restriction enzymes cut double-stranded or single stranded DNA at specific recognition nucleotide sequences known as restriction sites. In some embodiments, a rare-cutting restriction enzyme, i.e., enzymes with a long recognition site (at least 8 base pairs in length), is used to reduce the possibility of cleaving elsewhere in the capture probe.

In some embodiments, the cleavage domain includes a poly-U sequence which can be cleaved by a mixture of Uracil DNA glycosylase (UDG) and the DNA glycosylase-lyase Endonuclease VIII, commercially known as the USER™ enzyme. Releasable capture probes can be available for reaction once released. Thus, for example, an activatable capture probe can be activated by releasing the capture probes from a capture spot.

In some embodiments, where the capture probe is attached indirectly to a substrate, e.g., via a surface probe, the cleavage domain includes one or more mismatch nucleotides, so that the complementary parts of the surface probe and the capture probe are not 100% complementary (for example, the number of mismatched base pairs can one, two, or three base pairs). Such a mismatch is recognized, e.g., by the MutY and T7 endonuclease I enzymes, which results in cleavage of the nucleic acid molecule at the position of the mismatch.

In some embodiments, where the capture probe is attached to a capture spot indirectly, e.g., via a surface probe, the cleavage domain includes a nickase recognition site or sequence. Nickases are endonucleases which cleave only a single strand of a DNA duplex. Thus, the cleavage domain can include a nickase recognition site close to the 5′ end of the surface probe (and/or the 5′ end of the capture probe) such that cleavage of the surface probe or capture probe destabilises the duplex between the surface probe and capture probe thereby releasing the capture probe) from the capture spot.

Nickase enzymes can also be used in some embodiments where the capture probe is attached to the capture spot directly. For example, the substrate can be contacted with a nucleic acid molecule that hybridizes to the cleavage domain of the capture probe to provide or reconstitute a nickase recognition site, e.g., a cleavage helper probe. Thus, contact with a nickase enzyme will result in cleavage of the cleavage domain thereby releasing the capture probe from the capture spot. Such cleavage helper probes can also be used to provide or reconstitute cleavage recognition sites for other cleavage enzymes, e.g., restriction enzymes.

Some nickases introduce single-stranded nicks only at particular sites on a DNA molecule, by binding to and recognizing a particular nucleotide recognition sequence. A number of naturally-occurring nickases have been discovered, of which at present the sequence recognition properties have been determined for at least four. In general, any suitable nickase can be used to bind to a complementary nickase recognition site of a cleavage domain. Following use, the nickase enzyme can be removed from the assay or inactivated following release of the capture probes to prevent unwanted cleavage of the capture probes.

In some embodiments, a cleavage domain is absent from the capture probe.

In some embodiments, the region of the capture probe corresponding to the cleavage domain can be used for some other function. For example, an additional region for nucleic acid extension or amplification can be included where the cleavage domain would normally be positioned. In such embodiments, the region can supplement the functional domain or even exist as an additional functional domain. In some embodiments, the cleavage domain is present but its use is optional.

(iii) Functional Domain

Each capture probe can optionally include at least one functional domain. Each functional domain typically includes a functional nucleotide sequence for a downstream analytical step in the overall analysis procedure.

(iv) Spatial Barcode

As discussed above, the capture probe can include one or more spatial barcodes spatial barcodes. A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate.

A spatial barcode can be part of an analyte, or independent from an analyte (i.e., part of the capture probe). A spatial barcode can be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe.

Spatial barcodes can have a variety of different formats. For example, spatial barcodes can include polynucleotide spatial barcodes, random nucleic acid and/or amino acid sequences, and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before, during, and/or after sequencing of the sample. In some embodiments, a spatial barcode allows for identification and/or quantification of individual sequencing-reads. In some embodiments, a spatial barcode is used as a fluorescent barcode for which fluorescently labeled oligonucleotide probes hybridize to the spatial barcode.

In some embodiments, the spatial barcode is a nucleic acid sequence that does not substantially hybridize to analyte nucleic acid molecules in a biological sample. In some embodiments, the spatial barcode has less than 80% sequence identity to the nucleic acid sequences across a substantial part (e.g., 80% or more) of the nucleic acid molecules in the biological sample.

The spatial barcode sequences can include from about 6 to about 20 or more nucleotides within the sequence of the capture probes, but can include more. These nucleotides can be completely contiguous, i.e., in a single stretch of adjacent nucleotides, or they can be separated into two or more separate subsequences that are separated by 1 or more nucleotides. Separated spatial barcode subsequences can be from about 4 to about 16 nucleotides in length, but can be longer.

For multiple capture probes that are attached to a common array capture spot, the one or more spatial barcode sequences of the multiple capture probes can include sequences that are the same for all capture probes coupled to the capture spot, and/or sequences that are different across all capture probes coupled to the capture spot.

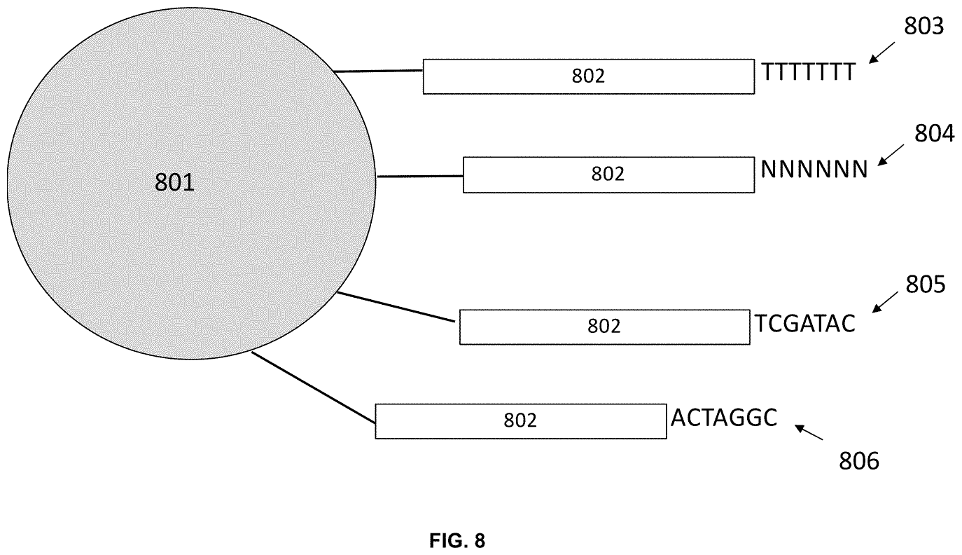

FIG. 8 is a schematic diagram of an exemplary multiplexed spatially-labelled capture spot. In FIG. 8, the capture spot 801 can be coupled to spatially-barcoded capture probes, where the spatially-barcoded probes of a particular capture spot can possess the same spatial barcode, but have different capture domains designed to associate the spatial barcode of the capture spot with more than one target analyte. For example, a capture spot may be coupled to four different types of spatially-barcoded capture probes, each type of spatially-barcoded capture probe possessing the spatial barcode 802. One type of capture probe associated with the capture spot includes the spatial barcode 802 in combination with a poly(T) capture domain 803, designed to capture mRNA target analytes. A second type of capture probe associated with the capture spot includes the spatial barcode 802 in combination with a random or degenerate N-mer capture domain 804 for gDNA analysis. A third type of capture probe associated with the capture spot includes the spatial barcode 802 in combination with a capture domain complementary to the capture domain on an analyte capture agent 805. A fourth type of capture probe associated with the capture spot includes the spatial barcode 802 in combination with a capture probe that can specifically bind a nucleic acid molecule 806 that can function in a CRISPR assay (e.g., CRISPR/Cas9). While only four different capture probe-barcoded constructs are shown in FIG. 8, capture-probe barcoded constructs can be tailored for analyses of any given analyte associated with a nucleic acid and capable of binding with such a construct. For example, the schemes shown in FIG. 8 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor).

Capture probes attached to a single array capture spot can include identical (or common) spatial barcode sequences, different spatial barcode sequences, or a combination of both. Capture probes attached to a capture spot can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences (e.g., nucleic acid barcode sequences) that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

(v) Unique Molecular Identifier

The capture probe can include one or more Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

(vi) Other Aspects of Capture Probes

For capture probes that are attached to an array capture spot, an individual array capture spot can include one or more capture probes. In some embodiments, an individual array capture spot includes hundreds, thousands, or millions of capture probes. In some embodiments, the capture probes are associated with a particular individual capture spot, where the individual capture spot contains a capture probe including a spatial barcode unique to a defined region or location on the array.

In some embodiments, a particular capture spot contains capture probes including more than one spatial barcode (e.g., one capture probe at a particular capture spot can include a spatial barcode that is different than the spatial barcode included in another capture probe at the same particular capture spot, while both capture probes include a second, common spatial barcode), where each spatial barcode corresponds to a particular defined region or location on the array. For example, multiple spatial barcode sequences associated with one particular capture spot on an array can provide a stronger address or attribution to a given location by providing duplicate or independent confirmation of the location. In some embodiments, the multiple spatial barcodes represent increasing specificity of the location of the particular array point. In a non-limiting example, a particular array point can be coded with two different spatial barcodes, where each spatial barcode identifies a particular defined region within the array, and an array point possessing both spatial barcodes identifies the sub-region where two defined regions overlap, e.g., such as the overlapping portion of a Venn diagram.

In another non-limiting example, a particular array point can be coded with three different spatial barcodes, where the first spatial barcode identifies a first region within the array, the second spatial barcode identifies a second region, where the second region is a subregion entirely within the first region, and the third spatial barcode identifies a third region, where the third region is a subregion entirely within the first and second subregions.

In some embodiments, capture probes attached to array capture spots are released from the array capture spots for sequencing. Alternatively, in some embodiments, capture probes remain attached to the array capture spots, and the probes are sequenced while remaining attached to the array capture spots (e.g., via in-situ sequencing). Further aspects of the sequencing of capture probes are described in subsequent sections of this disclosure.

In some embodiments, an array capture spot can include different types of capture probes attached to the capture spot. For example, the array capture spot can include a first type of capture probe with a capture domain designed to bind to one type of analyte, and a second type of capture probe with a capture domain designed to bind to a second type of analyte. In general, array capture spots can include one or more different types of capture probes attached to a single array capture spot.

In some embodiments, the capture probe is nucleic acid. In some embodiments, the capture probe is attached to the array capture spot via its 5′ end. In some embodiments, the capture probe includes from the 5′ to 3′ end: one or more barcodes (e.g., a spatial barcode and/or a UMI) and one or more capture domains. In some embodiments, the capture probe includes from the 5′ to 3′ end: one barcode (e.g., a spatial barcode or a UMI) and one capture domain. In some embodiments, the capture probe includes from the 5′ to 3′ end: a cleavage domain, a functional domain, one or more barcodes (e.g., a spatial barcode and/or a UMI), and a capture domain. In some embodiments, the capture probe includes from the 5′ to 3′ end: a cleavage domain, a functional domain, one or more barcodes (e.g., a spatial barcode and/or a UMI), a second functional domain, and a capture domain. In some embodiments, the capture probe includes from the 5′ to 3′ end: a cleavage domain, a functional domain, a spatial barcode, a UMI, and a capture domain. In some embodiments, the capture probe does not include a spatial barcode. In some embodiments, the capture probe does not include a UMI. In some embodiments, the capture probe includes a sequence for initiating a sequencing reaction.

In some embodiments, the capture probe is immobilized on a capture spot via its 3′ end. In some embodiments, the capture probe includes from the 3′ to 5′ end: one or more barcodes (e.g., a spatial barcode and/or a UMI) and one or more capture domains. In some embodiments, the capture probe includes from the 3′ to 5′ end: one barcode (e.g., a spatial barcode or a UMI) and one capture domain. In some embodiments, the capture probe includes from the 3′ to 5′ end: a cleavage domain, a functional domain, one or more barcodes (e.g., a spatial barcode and/or a UMI), and a capture domain. In some embodiments, the capture probe includes from the 3′ to 5′ end: a cleavage domain, a functional domain, a spatial barcode, a UMI, and a capture domain.

In some embodiments, a capture probe includes an in situ synthesized oligonucleotide. In some embodiments, the in situ synthesized oligonucleotide includes one or more constant sequences, one or more of which serves as a priming sequence (e.g., a primer for amplifying target nucleic acids). In some embodiments, a constant sequence is a cleavable sequence. In some embodiments, the in situ synthesized oligonucleotide includes a barcode sequence, e.g., a variable barcode sequence. In some embodiments, the in situ synthesized oligonucleotide is attached to a capture spot of an array.

In some embodiments, a capture probe is a product of two or more oligonucleotide sequences, e.g., two or more oligonucleotide sequences that are ligated together. In some embodiments, one of the oligonucleotide sequences is an in situ synthesized oligonucleotide.

In some embodiments, the capture probe includes a sequence that is complementary to a splint oligonucleotide. Two or more oligonucleotides can be ligated together using a splint oligonucleotide and any variety of ligases known in the art or described herein (e.g., SplintR ligase).

In some embodiments, one of the oligonucleotides includes: a constant sequence (e.g., a sequence complementary to a portion of a splint oligonucleotide), a degenerate sequence, and a capture domain (e.g., as described herein). In some embodiments, the capture probe is generated by having an enzyme add polynucleotides at the end of an oligonucleotide sequence. The capture probe can include a degenerate sequence, which can function as a unique molecular identifier.

A capture probe can include a degenerate sequence, which is a sequence in which some positions of a nucleotide sequence contain a number of possible bases. A degenerate sequence can be a degenerate nucleotide sequence including about five or more nucleotides. In some embodiments, a nucleotide sequence contains one or more degenerate positions within the nucleotide sequence. In some embodiments, the degenerate sequence is used as a UMI.

In some embodiments, a capture probe includes a restriction endonuclease recognition sequence or a sequence of nucleotides cleavable by specific enzyme activities, e.g., uracil. The capture probes can be subjected to an enzymatic cleavage, which removes the blocking domain and any of the additional nucleotides that are added to the 3′ end of the capture probe during the modification process. The removal of the blocking domain reveals and/or restores the free 3′ end of the capture domain of the capture probe. In some embodiments, additional nucleotides can be removed to reveal and/or restore the 3′ end of the capture domain of the capture probe.

In some embodiments, a blocking domain can be incorporated into the capture probe when it is synthesized, or after its synthesis. The terminal nucleotide of the capture domain is a reversible terminator nucleotide (e.g., 3′-O-blocked reversible terminator and 3′-unblocked reversible terminator), and can be included in the capture probe during or after probe synthesis.

(c) Substrate

For the spatial array-based analytical methods described in this section, the substrate functions as a support for direct or indirect attachment of capture probes to capture spots of the array. In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and that allows for positioning of biological samples, analytes, capture spots, and/or capture probes on the substrate.

A wide variety of different substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™ cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate.

The substrate can also correspond to a flow cell. Flow cells can be formed of any of the foregoing materials, and can include channels that permit reagents, solvents, capture spots, and molecules to pass through the cell.

Among the examples of substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In some embodiments, a substrate is coated with a surface treatment such as poly-L-lysine. Additionally or alternatively, the substrate can be treated by silanation, e.g., with epoxy-silane, amino-silane, and/or by a treatment with polyacrylamide.

The substrate can generally have any suitable form or format. For example, the substrate can be flat, curved, e.g., convexly or concavely curved towards the area where the interaction between a biological sample, e.g., tissue sample, and the substrate takes place. In some embodiments, the substrate is a flat, e.g., planar, chip, wafer, or slide. The substrate can contain one or more patterned surfaces within the substrate (e.g., channels, wells, projections, ridges, divots, etc.).

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

Substrates can optionally include various structures such as, but not limited to, projections, ridges, and channels. A substrate can be micropatterned to limit lateral diffusion (e.g., to prevent overlap of spatial barcodes). A substrate modified with such structures can be modified to allow association of analytes, capture spots (e.g., beads), or probes at individual sites. For example, the sites where a substrate is modified with various structures can be contiguous or non-contiguous with other sites.

In some embodiments, the surface of a substrate can be modified so that discrete sites are formed that can only have or accommodate a single capture spot. In some embodiments, the surface of a substrate can be modified so that capture spots adhere to random sites.

In some embodiments, the surface of a substrate is modified to contain one or more wells, using techniques such as (but not limited to) stamping techniques, microetching techniques, and molding techniques. In some embodiments in which a substrate includes one or more wells, the substrate can be a concavity slide or cavity slide. For example, wells can be formed by one or more shallow depressions on the surface of the substrate. In some embodiments, where a substrate includes one or more wells, the wells can be formed by attaching a cassette (e.g., a cassette containing one or more chambers) to a surface of the substrate structure.

In some embodiments, the structures of a substrate (e.g., wells) can each bear a different capture probe. Different capture probes attached to each structure can be identified according to the locations of the structures in or on the surface of the substrate. Exemplary substrates include arrays in which separate structures are located on the substrate including, for example, those having wells that accommodate capture spots.

In some embodiments, a substrate includes one or more markings on a surface of the substrate, e.g., to provide guidance for correlating spatial information with the characterization of the analyte of interest. For example, a substrate can be marked with a grid of lines (e.g., to allow the size of objects seen under magnification to be easily estimated and/or to provide reference areas for counting objects). In some embodiments, fiducial markers can be included on the substrate. Such markings can be made using techniques including, but not limited to, printing, sand-blasting, and depositing on the surface.

In some embodiments where the substrate is modified to contain one or more structures, including but not limited to wells, projections, ridges, or markings, the structures can include physically altered sites. For example, a substrate modified with various structures can include physical properties, including, but not limited to, physical configurations, magnetic or compressive forces, chemically functionalized sites, chemically altered sites, and/or electrostatically altered sites.

In some embodiments where the substrate is modified to contain various structures, including but not limited to wells, projections, ridges, or markings, the structures are applied in a pattern. Alternatively, the structures can be randomly distributed.

In some embodiments, a substrate is treated in order to minimize or reduce non-specific analyte hybridization within or between capture spots. For example, treatment can include coating the substrate with a hydrogel, film, and/or membrane that creates a physical barrier to non-specific hybridization. Any suitable hydrogel can be used.

Treatment can include adding a functional group that is reactive or capable of being activated such that it becomes reactive after receiving a stimulus (e.g., photoreactive). Treatment can include treating with polymers having one or more physical properties (e.g., mechanical, electrical, magnetic, and/or thermal) that minimize non-specific binding (e.g., that activate a substrate at certain locations to allow analyte hybridization at those locations).

The substrate (e.g., a bead or a capture spot on an array) can include tens to hundreds of thousands or millions of individual oligonucleotide molecules.

In some embodiments, the surface of the substrate is coated with a cell permissive coating to allow adherence of live cells. A “cell-permissive coating” is a coating that allows or helps cells to maintain cell viability (e.g., remain viable) on the substrate. For example, a cell-permissive coating can enhance cell attachment, cell growth, and/or cell differentiation, e.g., a cell-permissive coating can provide nutrients to the live cells. A cell-permissive coating can include a biological material and/or a synthetic material. Non-limiting examples of a cell-permissive coating include coatings that feature one or more extracellular matrix (ECM) components (e.g., proteoglycans and fibrous proteins such as collagen, elastin, fibronectin and laminin), poly-lysine, poly-L-ornithine, and/or a biocompatible silicone (e.g., CYTOSOFT®). For example, a cell-permissive coating that includes one or more extracellular matrix components can include collagen Type I, collagen Type II, collagen Type IV, elastin, fibronectin, laminin, and/or vitronectin. In some embodiments, the cell-permissive coating includes a solubilized basement membrane preparation extracted from the Engelbreth-Holm-Swarm (EHS) mouse sarcoma (e.g., MATRIGEL®). In some embodiments, the cell-permissive coating includes collagen.

Where the substrate includes a gel (e.g., a hydrogel or gel matrix), oligonucleotides within the gel can attach to the substrate. The terms “hydrogel” and “hydrogel matrix” are used interchangeably herein to refer to a macromolecular polymer gel including a network. Within the network, some polymer chains can optionally be cross-linked, although cross-linking does not always occur.

(d) Arrays

An “array” is an arrangement of a plurality of capture spots that is either irregular or forms a regular pattern. Individual capture spots in the array differ from one another based on their relative spatial locations. In general, at least two of the plurality of capture spots in the array include a distinct capture probe (e.g., any of the examples of capture probes described herein).

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given capture spot in the array. In some embodiments, a given capture spot in the array includes two or more species of oligonucleotides (e.g., capture probes). In some embodiments, the two or more species of oligonucleotides (e.g., capture probes) attached directly or indirectly to a given capture spot on the array include a common (e.g., identical) spatial barcode.

As defined above, a “capture spot” is an entity that acts as a support or repository for various molecular entities used in sample analysis. Examples of capture spots include, but are not limited to, a bead, a spot of any two- or three-dimensional geometry (e.g., an ink jet spot, a masked spot, a square on a grid), a well, and a hydrogel pad. In some embodiments, capture spots are directly or indirectly attached or fixed to a substrate. In some embodiments, the capture spots are not directly or indirectly attached or fixed to a substrate, but instead, for example, are disposed within an enclosed or partially enclosed three dimensional space (e.g., wells or divots).

In some embodiments, capture spots are directly or indirectly attached or fixed to a substrate that is liquid permeable. In some embodiments, capture spots are directly or indirectly attached or fixed to a substrate that is biocompatible. In some embodiments, capture spots are directly or indirectly attached or fixed to a substrate that is a hydrogel.

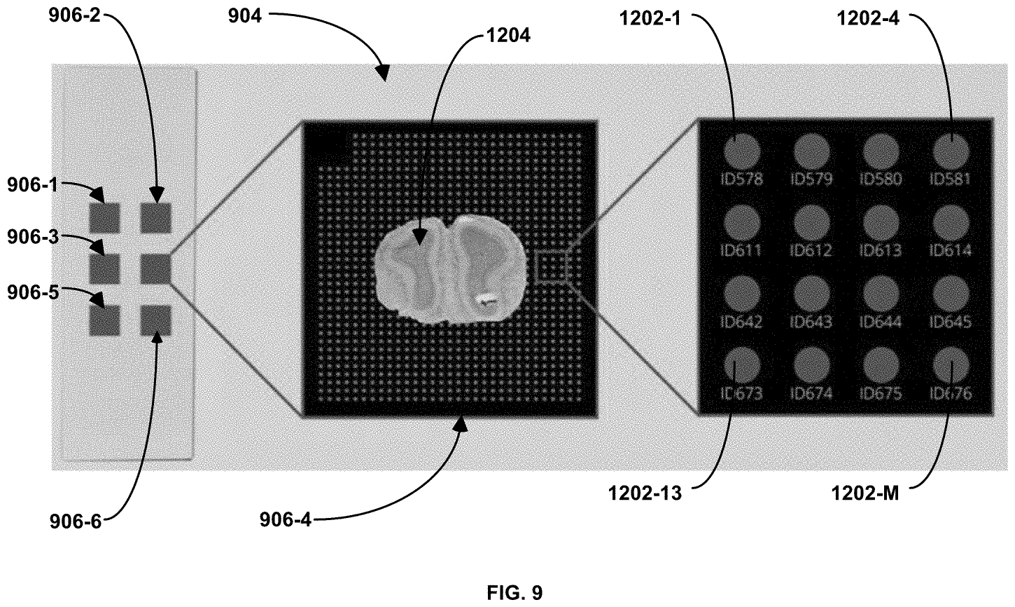

FIG. 9 depicts an exemplary arrangement of barcoded capture spots within an array. From left to right, FIG. 9 shows (L) a slide including six spatially-barcoded arrays, (C) an enlarged schematic of one of the six spatially-barcoded arrays 906-4, showing a grid of barcoded capture spots in relation to a biological sample, and (R) an enlarged schematic of one portion of an array, showing the specific identification of multiple capture spots within the array (labelled as ID578, ID579, ID580, etc.).

As used herein, the term “bead array” refers to an array that includes a plurality of beads as the capture spots in the array. In some embodiments, the beads are attached to a substrate. For example, the beads can optionally attach to a substrate such as a microscope slide and in proximity to a biological sample (e.g., a tissue section that includes cells). The beads can also be suspended in a solution and deposited on a surface (e.g., a membrane, a tissue section, or a substrate (e.g., a microscope slide)).

Examples of arrays of beads on or within a substrate include beads located in wells such as the BeadChip array (available from Illumina Inc., San Diego, Calif.), arrays used in sequencing platforms from 454 LifeSciences (a subsidiary of Roche, Basel, Switzerland), and array used in sequencing platforms from Ion Torrent (a subsidiary of Life Technologies, Carlsbad, Calif.).

In some embodiments, some or all capture spots in an array include a capture probe. In some embodiments, an array can include a capture probe attached directly or indirectly to the substrate.

The capture probe includes a capture domain (e.g., a nucleotide sequence) that can specifically bind (e.g., hybridize) to a target analyte (e.g., mRNA, DNA, or protein) within a sample. In some embodiments, the binding of the capture probe to the target (e.g., hybridization) can be detected and quantified by detection of a visual signal, e.g., a fluorophore, a heavy metal (e.g., silver ion), or chemiluminescent label, which has been incorporated into the target. In some embodiments, the intensity of the visual signal correlates with the relative abundance of each analyte in the biological sample. Since an array can contain thousands or millions of capture probes (or more), an array of capture spots with capture probes can interrogate many analytes in parallel.

In some embodiments, a substrate includes one or more capture probes that are designed to capture analytes from one or more organisms. In a non-limiting example, a substrate can contain one or more capture probes designed to capture mRNA from one organism (e.g., a human) and one or more capture probes designed to capture DNA from a second organism (e.g., a bacterium).

The capture probes can be attached to a substrate or capture spot using a variety of techniques. In some embodiments, the capture probe is directly attached to a capture spot that is fixed on an array. In some embodiments, the capture probes are immobilized to a substrate by chemical immobilization. For example, a chemical immobilization can take place between functional groups on the substrate and corresponding functional elements on the capture probes. Exemplary corresponding functional elements in the capture probes can either be an inherent chemical group of the capture probe, e.g., a hydroxyl group, or a functional element can be introduced on to the capture probe. An example of a functional group on the substrate is an amine group. In some embodiments, the capture probe to be immobilized includes a functional amine group or is chemically modified in order to include a functional amine group.

In some embodiments, the capture probe is a nucleic acid. In some embodiments, the capture probe is immobilized on the capture spot or the substrate via its 5′ end. In some embodiments, the capture probe is immobilized on a capture spot or a substrate via its 5′ end and includes from the 5′ to 3′ end: one or more barcodes (e.g., a spatial barcode and/or a UMI) and one or more capture domains. In some embodiments, the capture probe is immobilized on a capture spot via its 5′ end and includes from the 5′ to 3′ end: one barcode (e.g., a spatial barcode or a UMI) and one capture domain. In some embodiments, the capture probe is immobilized on a capture spot or a substrate via its 5′ end and includes from the 5′ to 3′ end: a cleavage domain, a functional domain, one or more barcodes (e.g., a spatial barcode and/or a UMI), and a capture domain.

In some embodiments, the capture probe is immobilized on a capture spot or a substrate via its 5′ end and includes from the 5′ to 3′ end: a cleavage domain, a functional domain, one or more barcodes (e.g., a spatial barcode and/or a UMI), a second functional domain, and a capture domain. In some embodiments, the capture probe is immobilized on a capture spot or a substrate via its 5′ end and includes from the 5′ to 3′ end: a cleavage domain, a functional domain, a spatial barcode, a UMI, and a capture domain. In some embodiments, the capture probe is immobilized on a capture spot or a substrate via its 5′ end and does not include a spatial barcode. In some embodiments, the capture probe is immobilized on a capture spot or a substrate via its 5′ end and does not include a UMI. In some embodiments, the capture probe includes a sequence for initiating a sequencing reaction.

In some embodiments, the capture probe is immobilized on a capture spot or a substrate via its 3′ end. In some embodiments, the capture probe is immobilized on a capture spot or a substrate via its 3′ end and includes from the 3′ to 5′ end: one or more barcodes (e.g., a spatial barcode and/or a UMI) and one or more capture domains. In some embodiments, the capture probe is immobilized on a capture spot or a substrate via its 3′ end and includes from the 3′ to 5′ end: one barcode (e.g., a spatial barcode or a UMI) and one capture domain. In some embodiments, the capture probe is immobilized on a capture spot or a substrate via its 3′ end and includes from the 3′ to 5′ end: a cleavage domain, a functional domain, one or more barcodes (e.g., a spatial barcode and/or a UMI), and a capture domain. In some embodiments, the capture probe is immobilized on a capture spot or a substrate via its 3′ end and includes from the 3′ to 5′ end: a cleavage domain, a functional domain, a spatial barcode, a UMI, and a capture domain.

The localization of the functional group within the capture probe to be immobilized can be used to control and shape the binding behavior and/or orientation of the capture probe, e.g., the functional group can be placed at the 5′ or 3′ end of the capture probe or within the sequence of the capture probe. In some embodiments, a capture probe can further include a support (e.g., a support attached to the capture probe, a support attached to the capture spot, or a support attached to the substrate). A typical support for a capture probe to be immobilized includes moieties which are capable of binding to such capture probes, e.g., to amine-functionalized nucleic acids. Examples of such supports are carboxy, aldehyde, or epoxy supports.

In some embodiments, the substrates on which capture probes can be immobilized can be chemically activated, e.g., by the activation of functional groups, available on the substrate. The term “activated substrate” relates to a material in which interacting or reactive chemical functional groups are established or enabled by chemical modification procedures. For example, a substrate including carboxyl groups can be activated before use. Furthermore, certain substrates contain functional groups that can react with specific moieties already present in the capture probes.

In some embodiments, a covalent linkage is used to directly couple a capture probe to a substrate. In some embodiments a capture probe is indirectly coupled to a substrate through a linker separating the “first” nucleotide of the capture probe from the support, i.e., a chemical linker. In some embodiments, a capture probe does not bind directly to the array, but interacts indirectly, for example by binding to a molecule which itself binds directly or indirectly to the array. In some embodiments, the capture probe is indirectly attached to a substrate (e.g., via a solution including a polymer).

In some embodiments, where the capture probe is immobilized on the capture spot of the array indirectly, e.g., via hybridization to a surface probe capable of binding the capture probe, the capture probe can further include an upstream sequence (5′ to the sequence that hybridizes to the nucleic acid, e.g., RNA of the tissue sample) that is capable of hybridizing to 5′ end of the surface probe. Alone, the capture domain of the capture probe can be seen as a capture domain oligonucleotide, which can be used in the synthesis of the capture probe in embodiments where the capture probe is immobilized on the array indirectly.

In some embodiments, a substrate is comprised of an inert material or matrix (e.g., glass slides) that has been functionalized by, for example, treatment with a material comprising reactive groups which enable immobilization of capture probes. Non-limiting examples include polyacrylamide hydrogels supported on an inert substrate (e.g., glass slide).

In some embodiments, functionalized biomolecules (e.g., capture probes) are immobilized on a functionalized substrate using covalent methods. Methods for covalent attachment include, for example, condensation of amines and activated carboxylic esters (e.g., N-hydroxysuccinimide esters); condensation of amine and aldehydes under reductive amination conditions; and cycloaddition reactions such as the Diels-Alder [4+2] reaction, 1,3-dipolar cycloaddition reactions, and [2+2] cycloaddition reactions. Methods for covalent attachment also include, for example, click chemistry reactions, including [3+2] cycloaddition reactions (e.g., Huisgen 1,3-dipolar cycloaddition reaction and copper(I)-catalyzed azide-alkyne cycloaddition (CuAAC)); thiol-ene reactions; the Diels-Alder reaction and inverse electron demand Diels-Alder reaction; [4+1]cycloaddition of isonitriles and tetrazines; and nucleophilic ring-opening of small carbocycles (e.g., epoxide opening with amino oligonucleotides). Methods for covalent attachment also include, for example, maleimides and thiols; and para-nitrophenyl ester-functionalized oligonucleotides and polylysine-functionalized substrate. Methods for covalent attachment also include, for example, disulfide reactions; radical reactions; and hydrazide-functionalized substrate (e.g., wherein the hydrazide functional group is directly or indirectly attached to the substrate) and aldehyde-functionalized oligonucleotides.

In some embodiments, functionalized biomolecules (e.g., capture probes) are immobilized on a functionalized substrate using photochemical covalent methods. Methods for photochemical covalent attachment include, for example, immobilization of antraquinone-conjugated oliognucleotides.

In some embodiments, functionalized biomolecules (e.g., capture probes) are immobilized on a functionalized substrate using non-covalent methods. Methods for non-covalent attachment include, for example, biotin-functionalized oligonucleotides and streptavidin-treated substrates.

In some embodiments, an oligonucleotide (e.g., a capture probe) can be attached to a substrate or capture spot.

In some embodiments, the surface of a substrate is coated with a cell permissive coating to facilitate adherence of live cells. A “cell-permissive coating” is a coating that allows or helps cells to maintain cell viability (e.g., remain viable) on the substrate. For example, a cell-permissive coating can enhance cell attachment, cell growth, and/or cell differentiation, e.g., a cell-permissive coating can provide nutrients to the live cells. A cell-permissive coating can include a biological material and/or a synthetic material. Non-limiting examples of a cell-permissive coating include coatings that feature one or more extracellular matrix (ECM) components (e.g., proteoglycans and fibrous proteins such as collagen, elastin, fibronectin and laminin), poly-lysine, poly-L-ornithine, and/or a biocompatible silicone (e.g., CYTOSOFT®). For example, a cell-permissive coating that includes one or more extracellular matrix components can include collagen Type I, collagen Type II, collagen Type IV, elastin, fibronectin, laminin, and/or vitronectin. In some embodiments, the cell-permissive coating includes a solubilized basement membrane preparation extracted from the Engelbreth-Holm-Swarm (EHS) mouse sarcoma (e.g., MATRIGEL®). In some embodiments, the cell-permissive coating includes collagen.

A “conditionally removable coating” is a coating that can be removed from the surface of a substrate upon application of a releasing agent. In some embodiments, a conditionally removable coating includes a hydrogel.

Arrays can be prepared by a variety of methods. In some embodiments, arrays are prepared through the synthesis (e.g., in-situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection.

In some embodiments, the arrays are “spotted” or “printed” with oligonucleotides and these oligonucleotides (e.g., capture probes) are then attached to the substrate. The oligonucleotides can be applied by either noncontact or contact printing. A noncontact printer can use the same method as computer printers (e.g., bubble jet or inkjet) to expel small droplets of probe solution onto the substrate. The specialized inkjet-like printer can expel nanoliter to picoliter volume droplets of oligonucleotide solution, instead of ink, onto the substrate. In contact printing, each print pin directly applies the oligonucleotide solution onto a specific location on the surface. The oligonucleotides can be attached to the substrate surface by the electrostatic interaction of the negative charge of the phosphate backbone of the DNA with a positively charged coating of the substrate surface or by UV-cross-linked covalent bonds between the thymidine bases in the DNA and amine groups on the treated substrate surface. In some embodiments, the substrate is a glass slide. In some embodiments, the oligonucleotides (e.g., capture probes) are attached to the substrate by a covalent bond to a chemical matrix, e.g., epoxy-silane, amino-silane, lysine, polyacrylamide, etc.

The arrays can also be prepared by in situ-synthesis. In some embodiments, these arrays can be prepared using photolithography. The method typically relies on UV masking and light-directed combinatorial chemical synthesis on a substrate to selectively synthesize probes directly on the surface of the array, one nucleotide at a time per spot, for many spots simultaneously. In some embodiments, a substrate contains covalent linker molecules that have a protecting group on the free end that can be removed by light. UV light is directed through a photolithographic mask to deprotect and activate selected sites with hydroxyl groups that initiate coupling with incoming protected nucleotides that attach to the activated sites. The mask is designed in such a way that the exposure sites can be selected, and thus specify the coordinates on the array where each nucleotide can be attached. The process can be repeated, a new mask is applied activating different sets of sites and coupling different bases, allowing arbitrary oligonucleotides to be constructed at each site. This process can be used to synthesize hundreds of thousands of different oligonucleotides. In some embodiments, maskless array synthesizer technology can be used. It uses an array of programmable micromirrors to create digital masks that reflect the desired pattern of UV light to deprotect the features.

In some embodiments, the inkjet spotting process can also be used for in-situ oligonucleotide synthesis. The different nucleotide precursors plus catalyst can be printed on the substrate, and are then combined with coupling and deprotection steps. This method relies on printing picoliter volumes of nucleotides on the array surface in repeated rounds of base-by-base printing that extends the length of the oligonucleotide probes on the array.

Arrays can also be prepared by active hybridization via electric fields to control nucleic acid transport. Negatively charged nucleic acids can be transported to specific sites, or capture spots, when a positive current is applied to one or more test sites on the array. The surface of the array can contain a binding molecule, e.g., streptavidin, which allows for the formation of bonds (e.g., streptavidin-biotin bonds) once electronically addressed biotinylated probes reach their targeted location. The positive current is then removed from the active capture spots, and new test sites can be activated by the targeted application of a positive current. The process are repeated until all sites on the array are covered.

An array for spatial analysis can be generated by various methods as described herein. In some embodiments, the array has a plurality of capture probes comprising spatial barcodes. These spatial barcodes and their relationship to the locations on the array can be determined. In some cases, such information is readily available, because the oligonucleotides are spotted, printed, or synthesized on the array with a predetermined pattern. In some cases, the spatial barcode can be decoded by methods described herein, e.g., by in-situ sequencing, by various labels associated with the spatial barcodes etc. In some embodiments, an array can be used as a template to generate a daughter array. Thus, the spatial barcode can be transferred to the daughter array with a known pattern.

In some embodiments, an array comprising barcoded probes can be generated through ligation of a plurality of oligonucleotides. In some instances, an oligonucleotide of the plurality contains a portion of a barcode, and the complete barcode is generated upon ligation of the plurality of oligonucleotides. For example, a first oligonucleotide containing a first portion of a barcode can be attached to a substrate (e.g., using any of the methods of attaching an oligonucleotide to a substrate described herein), and a second oligonucleotide containing a second portion of the barcode can then be ligated onto the first oligonucleotide to generate a complete barcode. Different combinations of the first, second and any additional portions of a barcode can be used to increase the diversity of the barcodes. In instances where the second oligonucleotide is also attached to the substrate prior to ligation, the first and/or the second oligonucleotide can be attached to the substrate via a surface linker which contains a cleavage site. Upon ligation, the ligated oligonucleotide is linearized by cleaving at the cleavage site.

To increase the diversity of the barcodes, a plurality of second oligonucleotides comprising two or more different barcode sequences can be ligated onto a plurality of first oligonucleotides that comprise the same barcode sequence, thereby generating two or more different species of barcodes. To achieve selective ligation, a first oligonucleotide attached to a substrate containing a first portion of a barcode can initially be protected with a protective group (e.g., a photocleavable protective group), and the protective group can be removed prior to ligation between the first and second oligonucleotide. In instances where the barcoded probes on an array are generated through ligation of two or more oligonucleotides, a concentration gradient of the oligonucleotides can be applied to a substrate such that different combinations of the oligonucleotides are incorporated into a barcoded probe depending on its location on the substrate.

Barcoded probes on an array can also be generated by adding single nucleotides to existing oligonucleotides on an array, for example, using polymerases that function in a template-independent manner. Single nucleotides can be added to existing oligonucleotides in a concentration gradient, thereby generating probes with varying length, depending on the location of the probes on the array.

Arrays can also be prepared by modifying existing arrays, for example, by modifying the oligonucleotides attached to the arrays. For instance, probes can be generated on an array that comprises oligonucleotides that are attached to the array at the 3′ end and have a free 5′ end. The oligonucleotides can be in situ synthesized oligonucleotides, and can include a barcode. The length of the oligonucleotides can be less than 50 nucleotides (nts). To generate probes using these oligonucleotides, a primer complementary to a portion of an oligonucleotide (e.g., a constant sequence shared by the oligonucleotides) can be used to hybridize with the oligonucleotide and extend (using the oligonucleotide as a template) to form a duplex and to create a 3′ overhang. The 3′ overhang thus allows additional nucleotides or oligonucleotides to be added on to the duplex. A capture probe can be generated by, for instance, adding one or more oligonucleotides to the end of the 3′ overhang (e.g., via splint oligonucleotide mediated ligation), where the added oligonucleotides can include the sequence or a portion of the sequence of a capture domain.

In some embodiments, a capture spot on the array includes a bead. In some embodiments, two or more beads are dispersed onto a substrate to create an array, where each bead is a capture spot on the array. Beads can optionally be dispersed into wells on a substrate, e.g., such that only a single bead is accommodated per well.

(i) Capture Spot Sizes

Capture spots on an array can be a variety of sizes. In some embodiments, a capture spot of an array has a diameter or maximum dimension between about 1 μm to 100 μm micrometers (e.g., 65 μm). In some embodiments, where the capture spot is a bead, the bead can have a diameter or maximum dimension no larger than 100 μm. In some embodiments, a plurality of beads has an average diameter no larger than 100 μm. In some embodiments, the volume of the bead can be 1 μm³ or greater. The capture spot can include one or more cross-sections that can be the same or different sizes (e.g., 0.0001 μm or greater). In some embodiments, capture spots can be of a nanometer scale (e.g., capture spots can have a diameter or maximum cross-sectional dimension of about 100 nanometers (nm) to about 900 nanometers (nm). In some embodiments, a capture spot has a diameter or size that is about the size of a single cell (e.g., a single cell under evaluation).

Capture spots can be of uniform size or heterogeneous size. “Polydispersity” generally refers to heterogeneity of sizes of molecules or particles. The polydispersity (PDI) can be calculated using the equation PDI=Mw/Mn, where Mw is the weight-average molar mass and Mn is the number-average molar mass. In certain embodiments, capture spots can be provided as a population or plurality of capture spots having a relatively monodisperse size distribution. Where it can be desirable to provide relatively consistent amounts of reagents, maintaining relatively consistent capture spot characteristics, such as size, can contribute to the overall consistency.

In some embodiments, the beads provided herein can have size distributions that have a coefficient of variation in their cross-sectional dimensions of less than 50%, less than 40%, less than 30%, less than 20%, less than 15%, less than 10%, less than 5%, or lower. In some embodiments, a plurality of beads provided herein has a polydispersity index of less than 50%, less than 45%, less than 40%, less than 35%, less than 30%, less than 25%, less than 20%, less than 15%, less than 10%, less than 5%, or lower.

(ii) Capture Spot Density

In some embodiments, an array comprises a plurality number of capture spots. In some embodiments, an array (e.g, two-dimensional array) includes between 4000 and 3,000,000 capture spots.

In some embodiments, the capture spots of the array can be arranged in a pattern. In some embodiments, the center of a capture spot of an array is between 1 μm and 100 μm from the center of another capture spot of the array. In some embodiments, the capture spots of an array can be uniformly positioned and the size and/or shape of a plurality of capture spots of an array can be approximately uniform.

In some embodiments, an array is approximately 8 mm by 8 mm. In some embodiments, an array is approximately 10 mm by 10 mm or larger.

In some embodiments, the array can be a high-density array. In some embodiments, the high-density array can be arranged in a pattern. In some embodiments, the high-density pattern of the array is produced by compacting or compressing capture spots together in one or more dimensions. In some embodiments, a high-density pattern may be created by spot printing or other techniques described herein. In some embodiments, the center of a capture spots of the array is between 50 μm and 120 μm from the center of another capture spot of the array. In some embodiments, the center of a capture spot of the array is between 55 μm and 115 μm, between 60 μm and 110 μm, 80 μm and 105 μm, or any range within the disclosed sub-ranges from the center of another capture spot of the array. In some embodiments, the center of a capture spot of the array is approximately 100 μm from the center of another capture spot of the array. In some embodiments, the center of a capture spot of the array is approximately 60 μm from the center of another capture spot of the array. In some embodiments, the number of capture spots in a single array is approximately 500,000 to 3,000,000 capture spots.

(iii) Array Resolution

As used herein, a “low resolution” array (e.g., a low resolution spatial array) refers to an array with capture spots having an average diameter of about 20 microns or greater. In some embodiments, substantially all (e.g., 80% or more) of the capture probes within a single capture spot include the same barcode (e.g., spatial barcode) such that upon deconvolution, resulting sequencing data from the detection of one or more analytes can be correlated with the spatial barcode of the capture spot, thereby identifying the location of the capture spot on the array, and thus determining the location of the one or more analytes in the biological sample.

A “high-resolution” array refers to an array with capture spots having an average diameter of about 1 micron to about 10 microns. This range in average diameter of capture spots corresponds to the approximate diameter of a single mammalian cell. Thus, a high-resolution spatial array is capable of detecting analytes at, or below, mammalian single-cell scale.

In some embodiments, resolution of an array can be improved by constructing an array with smaller capture spots. In some embodiments, resolution of an array can be improved by increasing the number of capture spots in the array. In some embodiments, the resolution of an array can be improved by packing capture spots closer together. For example, arrays including 5,000 capture spots were determined to provide higher resolution as compared to arrays including 1,000 capture spots (data not shown).

In some embodiments, the capture spots of the array may be arranged in a pattern, and in some cases, high-density pattern. In some embodiments, the high-density pattern of the array is produced by compacting or compressing capture spots together in one or more dimensions. In some embodiments, a high-density pattern may be created by spot printing or other techniques described herein. The number of median genes captures per cell and the median UMI counts per cell were higher when an array including 5,000 capture spots was used as compared to array including 1,000 capture spots (data not shown).

In some embodiments, an array includes a capture spot, where the capture spot incudes one or more capture probes (e.g., any of the capture probes described herein).

(e) Analyte Capture

In this section, general aspects of systems and methods for capturing analytes are described. Individual method steps and system features can be present in combination in many different embodiments; the specific combinations described herein do not in any way limit other combinations of steps and features.

Generally, analytes can be captured when contacting a biological sample with, e.g., a substrate comprising capture probes (e.g., substrate with capture probes embedded, spotted, printed on the substrate or a substrate with capture spots (e.g., beads, wells) comprising capture probes).

As used herein, “contact,” “contacted,” and/or “contacting,” a biological sample with a substrate comprising capture spots refers to any contact (e.g., direct or indirect) such that capture probes can interact (e.g., capture) with analytes from the biological sample. For example, the substrate may be near or adjacent to the biological sample without direct physical contact, yet capable of capturing analytes from the biological sample. In some embodiments the biological sample is in direct physical contact with the substrate. In some embodiments, the biological sample is in indirect physical contact with the substrate. For example, a liquid layer may be between the biological sample and the substrate. In some embodiments, the analytes diffuse through the liquid layer. In some embodiments the capture probes diffuse through the liquid layer. In some embodiments reagents may be delivered via the liquid layer between the biological sample and the substrate. In some embodiments, indirect physical contact may be the presence of a second substrate (e.g., a hydrogel, a film, a porous membrane) between the biological sample and the first substrate comprising capture spots with capture probes. In some embodiments, reagents may be delivered by the second substrate to the biological sample.

(i) Diffusion-Resistant Media/Lids

To increase efficiency by encouraging analyte diffusion toward the spatially-labelled capture probes, a diffusion-resistant medium can be used. In general, molecular diffusion of biological analytes occurs in all directions, including toward the capture probes (i.e. toward the spatially-barcoded array), and away from the capture probes (i.e. into the bulk solution). Increasing diffusion toward the spatially-barcoded array reduces analyte diffusion away from the spatially-barcoded array and increases the capturing efficiency of the capture probes.

In some embodiments, a biological sample is placed on the top of a spatially-barcoded substrate and a diffusion-resistant medium is placed on top of the biological sample. For example, the diffusion-resistant medium can be placed onto an array that has been placed in contact with a biological sample. In some embodiments, the diffusion-resistant medium and spatially-labelled array are the same component. For example, the diffusion-resistant medium can contain spatially-labelled capture probes within or on the diffusion-resistant medium (e.g., coverslip, slide, hydrogel, or membrane). In some embodiments, a sample is placed on a support and a diffusion-resistant medium is placed on top of the biological sample. Additionally, a spatially-barcoded capture probe array can be placed in close proximity over the diffusion-resistant medium. For example, a diffusion-resistant medium may be sandwiched between a spatially-labelled array and a sample on a support. In some embodiments, the diffusion-resistant medium is disposed or spotted onto the sample. In other embodiments, the diffusion-resistant medium is placed in close proximity to the sample.

In general, the diffusion-resistant medium can be any material known to limit diffusivity of biological analytes. For example, the diffusion-resistant medium can be a solid lid (e.g., coverslip or glass slide). In some embodiments, the diffusion-resistant medium may be made of glass, silicon, paper, hydrogel polymer monoliths, or other material. In some embodiments, the glass side can be an acrylated glass slide. In some embodiments, the diffusion-resistant medium is a porous membrane. In some embodiments, the material may be naturally porous. In some embodiments, the material may have pores or wells etched into solid material. In some embodiments, the pore size can be manipulated to minimize loss of target analytes. In some embodiments, the membrane chemistry can be manipulated to minimize loss of target analytes. In some embodiments, the diffusion-resistant medium (i.e. hydrogel) is covalently attached to a solid support (i.e. glass slide). In some embodiments, the diffusion-resistant medium can be any material known to limit diffusivity of polyA transcripts. In some embodiments, the diffusion-resistant medium can be any material known to limit the diffusivity of proteins. In some embodiments, the diffusion-resistant medium can be any material know to limit the diffusivity of macromolecular constituents.

In some embodiments, a diffusion-resistant medium includes one or more diffusion-resistant media. For example, one or more diffusion-resistant media can be combined in a variety of ways prior to placing the media in contact with a biological sample including, without limitation, coating, layering, or spotting. As another example, a hydrogel can be placed onto a biological sample followed by placement of a lid (e.g., glass slide) on top of the hydrogel.

In some embodiments, a force (e.g., hydrodynamic pressure, ultrasonic vibration, solute contrasts, microwave radiation, vascular circulation, or other electrical, mechanical, magnetic, centrifugal, and/or thermal forces) is applied to control diffusion and enhance analyte capture. In some embodiments, one or more forces and one or more diffusion-resistant media are used to control diffusion and enhance capture. For example, a centrifugal force and a glass slide can used contemporaneously. Any of a variety of combinations of a force and a diffusion-resistant medium can be used to control or mitigate diffusion and enhance analyte capture.

In some embodiments, the diffusion-resistant medium, along with the spatially-barcoded array and sample, is submerged in a bulk solution. In some embodiments, the bulk solution includes permeabilization reagents. In some embodiments, the diffusion-resistant medium includes at least one permeabilization reagent. In some embodiments, the diffusion-resistant medium (i.e. hydrogel) is soaked in permeabilization reagents before contacting the diffusion-resistant medium to the sample. In some embodiments, the diffusion-resistant medium can include wells (e.g., micro-, nano-, or picowells) containing a permeabilization buffer or reagents. In some embodiments, the diffusion-resistant medium can include permeabilization reagents. In some embodiments, the diffusion-resistant medium can contain dried reagents or monomers to deliver permeabilization reagents when the diffusion-resistant medium is applied to a biological sample. In some embodiments, the diffusion-resistant medium is added to the spatially-barcoded array and sample assembly before the assembly is submerged in a bulk solution.

In some embodiments, the diffusion-resistant medium is added to the spatially-barcoded array and sample assembly after the sample has been exposed to permeabilization reagents. In some embodiments, the permeabilization reagents are flowed through a microfluidic chamber or channel over the diffusion-resistant medium. In some embodiments, the flow controls the sample's access to the permeabilization reagents. In some embodiments, the target analytes diffuse out of the sample and toward a bulk solution and get embedded in a spatially-labelled capture probe-embedded diffusion-resistant medium.

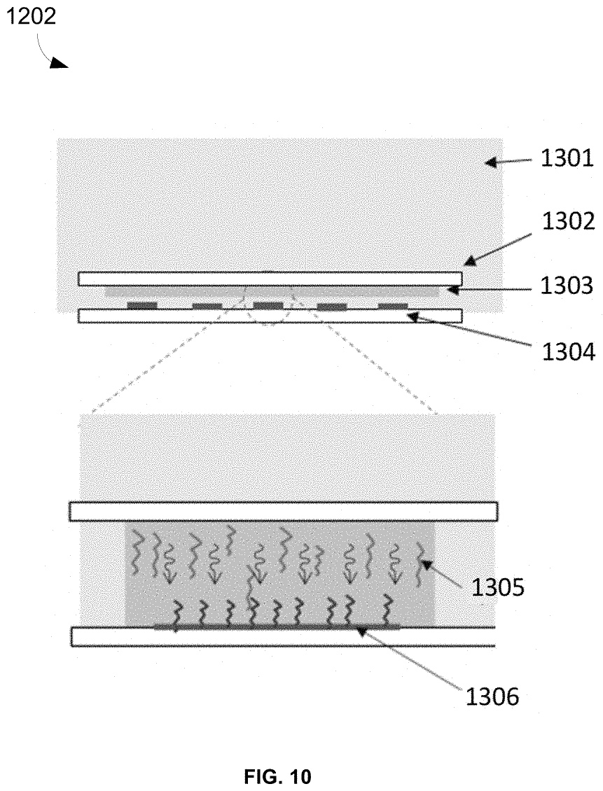

FIG. 10 is an illustration of an exemplary use of a diffusion-resistant medium. A diffusion-resistant medium 1302 can be contacted with a sample 1303. In FIG. 10, a glass slide 1304 is populated with spatially-barcoded capture probes 1306, and the sample 1303, 1305 is contacted with the array 1304, 1306. A diffusion-resistant medium 1302 can be applied to the sample 1303, wherein the sample 1303 is sandwiched between a diffusion-resistant medium 1302 and a capture probe coated slide 1304. When a permeabilization solution 1301 is applied to the sample, using the diffusion-resistant medium/lid 1302 directs migration of the analytes 1305 toward the capture probes 1306 by reducing diffusion of the analytes out into the medium. Alternatively, the lid may contain permeabilization reagents.

(ii) Conditions for Capture

Capture probes on the substrate (or on a capture spot on the substrate) interact with released analytes through a capture domain, described elsewhere, to capture analytes. In some embodiments, certain steps are performed to enhance the transfer or capture of analytes by the capture probes of the array. Examples of such modifications include, but are not limited to, adjusting conditions for contacting the substrate with a biological sample (e.g., time, temperature, orientation, pH levels, pre-treating of biological samples, etc.), using force to transport analytes (e.g., electrophoretic, centrifugal, mechanical, etc.), performing amplification reactions to increase the amount of biological analytes (e.g., PCR amplification, in situ amplification, clonal amplification), and/or using labeled probes for detecting of amplicons and barcodes.

In some embodiments, capture of analytes is facilitated by treating the biological sample with permeabilization reagents. If a biological sample is not permeabilized sufficiently, the amount of analyte captured on the substrate can be too low to enable adequate analysis. Conversely, if the biological sample is too permeable, the analyte can diffuse away from its origin in the biological sample, such that the relative spatial relationship of the analytes within the biological sample is lost. Hence, a balance between permeabilizing the biological sample enough to obtain good signal intensity while still maintaining the spatial resolution of the analyte distribution in the biological sample is desired. Methods of preparing biological samples to facilitation are known in the art and can be modified depending on the biological sample and how the biological sample is prepared (e.g., fresh frozen, FFPE, etc.).

(iii) Passive Capture Methods

In some embodiments, analytes are migrated from a sample to a substrate. Methods for facilitating migration can be passive (e.g., diffusion) and/or active (e.g., electrophoretic migration of nucleic acids). Non-limiting examples of passive migration can include simple diffusion and osmotic pressure created by the rehydration of dehydrated objects.

Passive migration by diffusion uses concentration gradients. Diffusion is movement of untethered objects toward equilibrium. Therefore, when there is a region of high object concentration and a region of low object concentration, the object (capture probe, the analyte, etc.) moves to an area of lower concentration. In some embodiments, untethered analytes move down a concentration gradient.

In some embodiments, different reagents are added to the biological sample, such that the biological sample is rehydrated while improving capture of analytes. In some embodiments, the biological sample is rehydrated with permeabilization reagents. In some embodiments, the biological sample is rehydrated with a staining solution (e.g., hematoxylin and eosin stain).

(iv) Active Capture Methods

In some examples of any of the methods described herein, an analyte in a cell or a biological sample can be transported (e.g., passively or actively) to a capture probe (e.g., a capture probe affixed to a solid surface).

For example, analytes in a cell or a biological sample can be transported to a capture probe (e.g., an immobilized capture probe) using an electric field (e.g., using electrophoresis), a pressure gradient, fluid flow, a chemical concentration gradient, a temperature gradient, and/or a magnetic field. For example, analytes can be transported through, e.g., a gel (e.g., hydrogel matrix), a fluid, or a permeabilized cell, to a capture probe (e.g., an immobilized capture probe).

In some examples, an electrophoretic field can be applied to analytes to facilitate migration of the analytes towards a capture probe. In some examples, a sample contacts a substrate and capture probes fixed on a substrate (e.g., a slide, cover slip, or bead), and an electric current is applied to promote the directional migration of charged analytes towards the capture probes fixed on the substrate. An electrophoresis assembly, where a cell or a biological sample is in contact with a cathode and capture probes (e.g., capture probes fixed on a substrate), and where the capture probes (e.g., capture probes fixed on a substrate) is in contact with the cell or biological sample and an anode, can be used to apply the current.

Electrophoretic transfer of analytes can be performed while retaining the relative spatial alignment of the analytes in the sample. As such, an analyte captured by the capture probes (e.g., capture probes fixed on a substrate) retains the spatial information of the cell or the biological sample.

In some examples, a spatially-addressable microelectrode array is used for spatially-constrained capture of at least one charged analyte of interest by a capture probe. The microelectrode array can be configured to include a high density of discrete sites having a small area for applying an electric field to promote the migration of charged analyte(s) of interest. For example, electrophoretic capture can be performed on a region of interest using a spatially-addressable microelectrode array.

(v) Region of Interest

A biological sample can have regions that show morphological feature(s) that may indicate the presence of disease or the development of a disease phenotype. For example, morphological features at a specific site within a tumor biopsy sample can indicate the aggressiveness, therapeutic resistance, metastatic potential, migration, stage, diagnosis, and/or prognosis of cancer in a subject. A change in the morphological features at a specific site within a tumor biopsy sample often correlate with a change in the level or expression of an analyte in a cell within the specific site, which can, in turn, be used to provide information regarding the aggressiveness, therapeutic resistance, metastatic potential, migration, stage, diagnosis, and/or prognosis of cancer in a subject. A region or area within a biological sample that is selected for specific analysis (e.g., a region in a biological sample that has morphological features of interest) is often described as “a region of interest.”

A region of interest in a biological sample can be used to analyze a specific area of interest within a biological sample, and thereby, focus experimentation and data gathering to a specific region of a biological sample (rather than an entire biological sample). This results in increased time efficiency of the analysis of a biological sample.

A region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, and confocal microscopy, and combinations thereof. For example, the staining and imaging of a biological sample can be performed to identify a region of interest. In some examples, the region of interest can correspond to a specific structure of cytoarchitecture. In some embodiments, a biological sample can be stained prior to visualization to provide contrast between the different regions of the biological sample. The type of stain can be chosen depending on the type of biological sample and the region of the cells to be stained. In some embodiments, more than one stain can be used to visualize different aspects of the biological sample, e.g., different regions of the sample, specific cell structures (e.g., organelles), or different cell types. In other embodiments, the biological sample can be visualized or imaged without staining the biological sample.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, calorimetric, and colorimetric labels.

In some embodiments, a fiducial marker can be present on a substrate to provide orientation of the biological sample. In some embodiments, a microsphere can be coupled to a substrate to aid in orientation of the biological sample. In some examples, a microsphere coupled to a substrate can produce an optical signal (e.g., fluorescence). In another example, a microsphere can be attached to a portion (e.g., corner) of an array in a specific pattern or design (e.g., hexagonal design) to aid in orientation of a biological sample on an array of capture spots on the substrate. In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Such a marker nucleic acid molecule can be contacted with an array before, contemporaneously with, or after the tissue sample is stained to visualize or image the tissue section. Although not required, it can be advantageous to use a marker that can be detected using the same conditions (e.g., imaging conditions) used to detect a labelled cDNA.

In some embodiments, fiducial markers are included to facilitate the orientation of a tissue sample or an image thereof in relation to an immobilized capture probes on a substrate. Any number of methods for marking an array can be used such that a marker is detectable only when a tissue section is imaged. For instance, a molecule, e.g., a fluorescent molecule that generates a signal, can be immobilized directly or indirectly on the surface of a substrate. Markers can be provided on a substrate in a pattern (e.g., an edge, one or more rows, one or more lines, etc.).

In some embodiments, a fiducial marker can be randomly placed in the field of view. For example, an oligonucleotide containing a fluorophore can be randomly printed, stamped, synthesized, or attached to a substrate (e.g., a glass slide) at a random position on the substrate. A tissue section can be contacted with the substrate such that the oligonucleotide containing the fluorophore contacts, or is in proximity to, a cell from the tissue section or a component of the cell (e.g., an mRNA or DNA molecule). An image of the substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a substrate). In this instance, a fiducial marker can be stamped, attached, or synthesized on the substrate and contacted with a biological sample. Typically, an image of the sample and the fiducial marker is taken, and the position of the fiducial marker on the substrate can be confirmed by viewing the image.

In some examples, fiducial markers can surround the array. In some embodiments the fiducial markers allow for detection of, e.g., mirroring. In some embodiments, the fiducial markers may completely surround the array. In some embodiments, the fiducial markers may not completely surround the array. In some embodiments, the fiducial markers identify the corners of the array. In some embodiments, one or more fiducial markers identify the center of the array. In some embodiments, the fiducial markers comprise patterned spots, wherein the diameter of one or more patterned spot fiducial markers is approximately 100 micrometers. The diameter of the fiducial markers can be any useful diameter including, but not limited to, 50 micrometers to 500 micrometers in diameter. The fiducial markers may be arranged in such a way that the center of one fiducial marker is between 100 micrometers and 200 micrometers from the center of one or more other fiducial markers surrounding the array. In some embodiments, the array with the surrounding fiducial markers is approximately 8 mm by 8 mm. In some embodiments, the array without the surrounding fiducial markers is smaller than 8 mm by 8 mm. In some embodiments, the array without the surrounding fiducial markers is larger than 10 mm by 10 mm.

In some embodiments, staining and imaging a biological sample prior to contacting the biological sample with a spatial array is performed to select samples for spatial analysis. In some embodiments, the staining includes applying a fiducial marker as described above, including fluorescent, radioactive, chemiluminescent, calorimetric, or colorimetric detectable markers. In some embodiments, the staining and imaging of biological samples allows the user to identify the specific sample (or region of interest) the user wishes to assess.

In some embodiments, a lookup table (LUT) can be used to associate one property with another property of a capture spot. These properties include, e.g., locations, barcodes (e.g., nucleic acid barcode molecules), spatial barcodes, optical labels, molecular tags, and other properties.

In some embodiments, a lookup table can associate a nucleic acid barcode molecule with a capture spot. In some embodiments, an optical label of a capture spot can permit associating the capture spot with a biological particle (e.g., cell or nuclei). The association of a capture spot with a biological particle can further permit associating a nucleic acid sequence of a nucleic acid molecule of the biological particle to one or more physical properties of the biological particle (e.g., a type of a cell or a location of the cell). For example, based on the relationship between the barcode and the optical label, the optical label can be used to determine the location of a capture spot, thus associating the location of the capture spot with the barcode sequence of the capture spot. Subsequent analysis (e.g., sequencing) can associate the barcode sequence and the analyte from the sample. Accordingly, based on the relationship between the location and the barcode sequence, the location of the biological analyte can be determined (e.g., in a specific type of cell or in a cell at a specific location of the biological sample).

In some embodiments, a capture spot can have a plurality of nucleic acid barcode molecules attached thereto. The plurality of nucleic acid barcode molecules can include barcode sequences. The plurality of nucleic acid molecules attached to a given capture spot can have the same barcode sequences, or two or more different barcode sequences. Different barcode sequences can be used to provide improved spatial location accuracy.

In some embodiments, a substrate is treated in order to minimize or reduce non-specific analyte hybridization within or between capture spots. For example, treatment can include coating the substrate with a hydrogel, film, and/or membrane that creates a physical barrier to non-specific hybridization. Any suitable hydrogel can be used.

Treatment can include adding a functional group that is reactive or capable of being activated such that it becomes reactive after receiving a stimulus (e.g., photoreactive). Treatment can include treating with polymers having one or more physical properties (e.g., mechanical, electrical, magnetic, and/or thermal) that minimize non-specific binding (e.g., that activate a substrate at certain locations to allow analyte hybridization at those locations).

In some examples, an array (e.g., any of the exemplary arrays described herein) can be contained with only a portion of a biological sample (e.g., a cell, a feature, or a region of interest). In some examples, a biological sample is contacted with only a portion of an array (e.g., any of the exemplary arrays described herein). In some examples, a portion of the array can be deactivated such that it does not interact with the analytes in the biological sample (e.g., optical deactivation, chemical deactivation, heat deactivation, or blocking of the capture probes in the array (e.g., using blocking probes)). In some examples, a region of interest can be removed from a biological sample and then the region of interest can be contacted to the array (e.g., any of the arrays described herein). A region of interest can be removed from a biological sample using microsurgery, laser capture microdissection, chunking, a microtome, dicing, trypsinization, labelling, and/or fluorescence-assisted cell sorting.

(f) Analysis of Captured Analytes

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a method for spatially detecting an analyte (e.g., detecting the location of an analyte, e.g., a biological analyte) from a biological sample (e.g., present in a biological sample), comprises: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the support.

In some embodiments, a method for spatially detecting an analyte (e.g., detecting the location of an analyte, e.g., a biological analyte) from a biological sample (e.g., present in a biological sample) is provides that comprises: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, a method for spatially detecting a biological analyte of interest from a biological sample is provided that comprises: (a) staining and imaging a biological sample on a support; (b) providing a solution comprising a permeabilization reagent to the biological sample on the support; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the support.

In some embodiments, the method further includes selecting a region of interest in the biological sample to subject to spatial transcriptomic analysis. In some embodiments, one or more of the one or more capture probes include a capture domain. In some embodiments, one or more of the one or more capture probe pluralities comprise a unique molecular identifier (UMI). In some embodiments, one or more of the one or more capture probe pluralities comprise a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

After analytes from the sample have hybridized or otherwise been associated with capture probes, analyte capture agents, or other barcoded oligonucleotide sequences according to any of the methods described above in connection with the general spatial cell-based analytical methodology, the barcoded constructs that result from hybridization/association are analyzed via sequencing to identify the analytes.

In some embodiments, the methods described herein can be used to assess analyte levels and/or expression in a cell or a biological sample over time (e.g., before or after treatment with an agent or different stages of differentiation). In some examples, the methods described herein can be performed on multiple similar biological samples or cells obtained from the subject at a different time points (e.g., before or after treatment with an agent, different stages of differentiation, different stages of disease progression, different ages of the subject, or before or after development of resistance to an agent).

### III. Specific Embodiments of Systems and Methods for Binary Tissue Classification

This disclosure also provides methods and systems for binary tissue classification. Provided below are detailed descriptions and explanations of various embodiments of the present disclosure. These embodiments are non-limiting and do not preclude any alternatives, variations, changes, and substitutions that can occur to those skilled in the art from the scope of this disclosure.

(a) Systems for Binary Tissue Classification

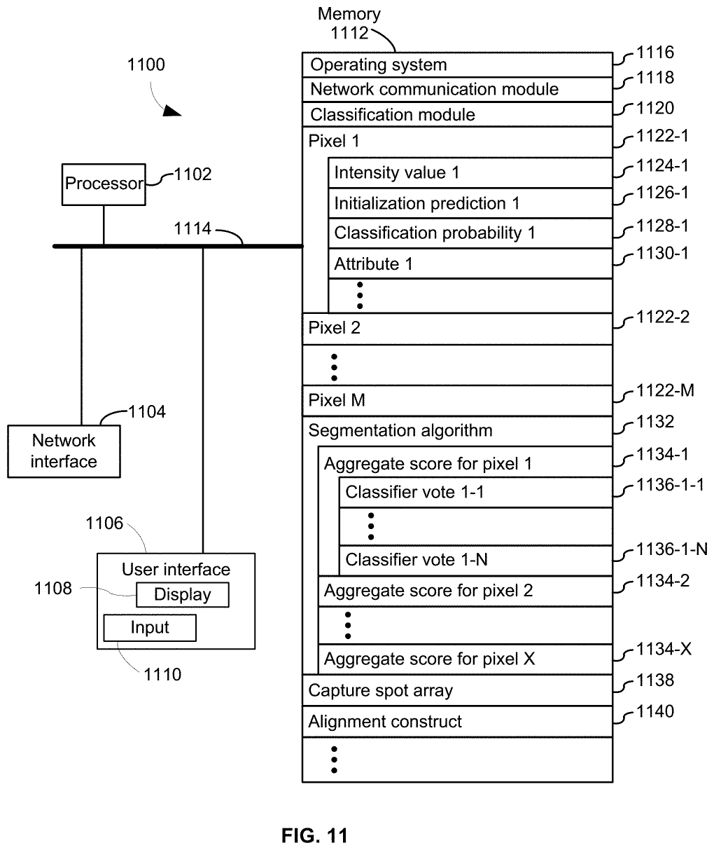

FIG. 11 is a block diagram illustrating an exemplary, non-limiting system for binary tissue classification in accordance with some implementations. The system 1100 in some implementations includes one or more processors 1102, one or more network interfaces 1104, a user interface 1106, a memory 1112, and one or more communication buses 1114 for interconnecting these components. In some embodiments, the one or more processors 1102 may be implemented with one or more graphics processing units (GPUs), with each GPU comprising a plurality of processing cores (e.g., thousands).

The communication buses 1114 optionally include circuitry (sometimes called a chipset) that interconnects and controls communications between system components. The memory 1112 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, other random access solid state memory devices, or any other medium which can be used to store desired information; and optionally includes non-volatile memory, such as one or more magnetic disk storage devices, optical disk storage devices, flash memory devices, or other non-volatile solid state storage devices. The memory 1112 optionally includes one or more storage devices remotely located from the processor 1102. The memory 1112, or alternatively the non-volatile memory device(s) within the memory 1112, comprises a non-transitory computer readable storage medium. In some implementations, the memory 1112 or alternatively the non-transitory computer readable storage medium stores the following programs, modules and data structures, or a subset thereof:
an optional operating system 1116 that includes procedures for handling various basic system services and for performing hardware dependent tasks;
an optional network communication module (or instructions) 1118 for connecting the device 1100 with other devices, or a communication network;
an optional classification module 1120 for classifying a pixel of an image as tissue or background;
a plurality of pixels in electronic form 1122-1, 1122-2, . . . , 1122-M, each respective pixel 1122 in the plurality of pixels in an image of a sectioned tissue sample overlayed on a substrate, from a subject, obtained using transmission light microscopy and each respective pixel 1122 in the plurality of pixels associated with at least a pixel intensity value 1124 (e.g., 1124-1), an initialization prediction 1126 (e.g., 1126-1), a classification probability 1128 (e.g., 1128-1), and an optional attribute 1130 (e.g., 1130-1);
a segmentation algorithm 1132 comprising a plurality of aggregate scores 1134-1, 1134-2, . . . , 1134-X, each aggregate score for a respective pixel in the plurality of pixels and comprising a plurality of classifier votes 1136 (e.g., 1136-1-1, . . . , 1136-1-N) obtained using one or more heuristic classifiers;
an optional capture spot array 1138 comprising a representation of a set of capture spots in the form of a two-dimensional array of positions on the substrate, each respective capture spot at a different position in the two-dimensional array and associating with one or more analytes from the tissue, and each respective capture spot characterized by at least one different corresponding spatial barcode in a plurality of spatial barcodes; and
an optional alignment construct 1140 for assigning each respective representation of a capture spot in the plurality of capture spots in the optional capture spot array 1138 with a first attribute or a second attribute 1130 based upon the assignment of pixels in the vicinity of the respective representation of the capture spot in the image.

For example, in some implementations of the present disclosure, the pixel intensity value 1124 for a respective pixel 1122 (e.g., pixel 1) in the plurality of pixels is used by one or more heuristic classifiers that, in turn, provide one or more classifier votes in a plurality of classifier votes 1136. The plurality of classifier votes 1136-1-1, . . . , 1136-1-N, forms an aggregated score 1134 for the respective pixel, which is then used to determine an initialization prediction 1126 (e.g., obvious first class, likely first class, likely second class, obvious second class) for the respective pixel 1122. In some implementations, the segmentation algorithm 1132 subsequently uses the pixel intensity value 1124 and the initialization prediction 1126 for a respective pixel 1122 to assign a classification probability 1128, indicating whether the pixel exhibits a greater probability of representing tissue or, conversely, a greater probability of representing background. In some optional implementations, the classification probability 1128 of a respective pixel 1122 determines whether the pixel is assigned a first or second attribute 1130 that is represented as a tissue mask overlayed on the image for each pixel in the plurality of pixels. Finally, in some optional implementations, the tissue mask containing a first or second attribute 1130 for each pixel in the plurality of pixels is further assigned to a capture spot array 1138, for each capture spot in the capture spot array 1138 in the vicinity of the respective pixel that is determined using an optional alignment construct 1140.

In some implementations, the user interface 1106 includes an input device (e.g., a keyboard, a mouse, a touchpad, a track pad, and/or a touch screen) 1110 for a user to interact with the system 1100 and a display 1108.

In some implementations, one or more of the above identified elements are stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 1112 optionally stores a subset of the modules and data structures identified above. Furthermore, in some embodiments, the memory stores additional modules and data structures not described above. In some embodiments, one or more of the above identified elements is stored in a computer system, other than that of system 1100, that is addressable by system 1100 so that system 1100 may retrieve all or a portion of such data when needed.

Although FIG. 11 shows an exemplary system 1100, the figure is intended more as a functional description of the various features that may be present in computer systems than as a structural schematic of the implementations described herein. In practice, and as recognized by those of ordinary skill in the art, items shown separately could be combined and some items could be separated.

(b) Methods for Binary Tissue Classification

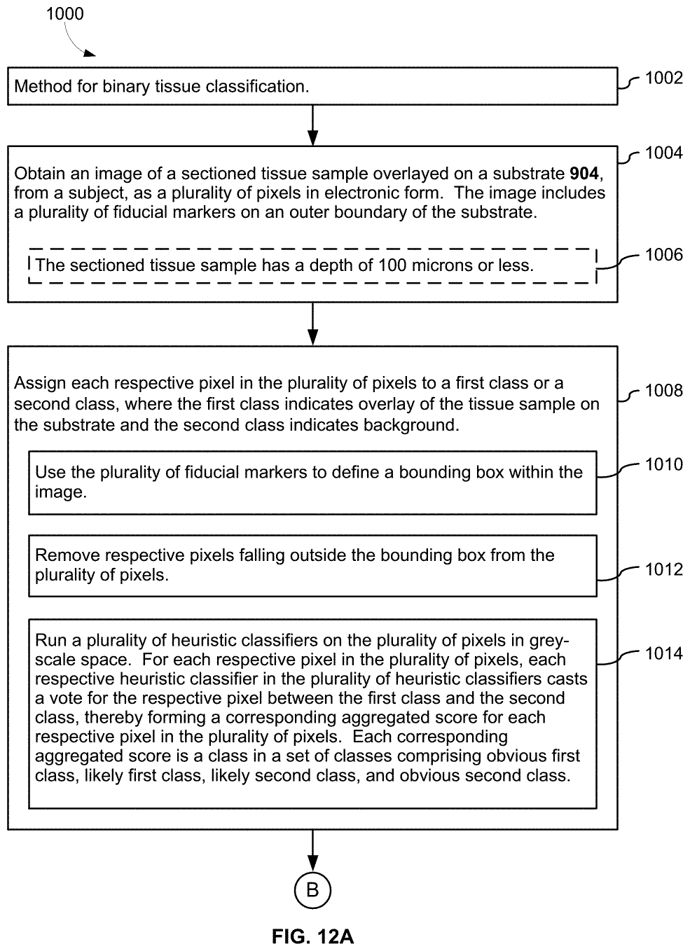

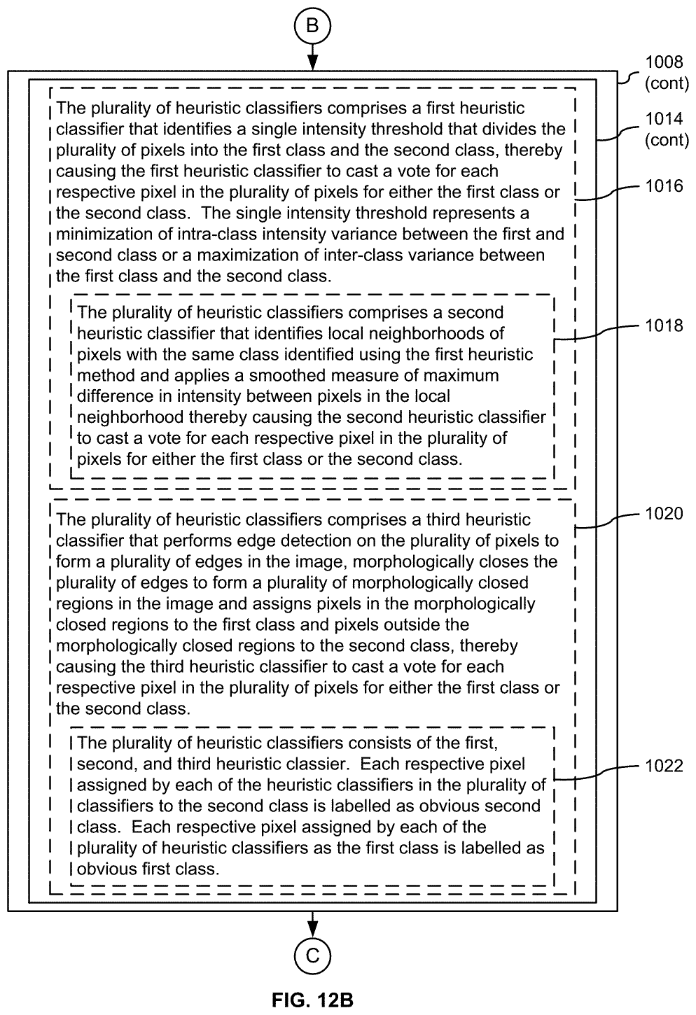

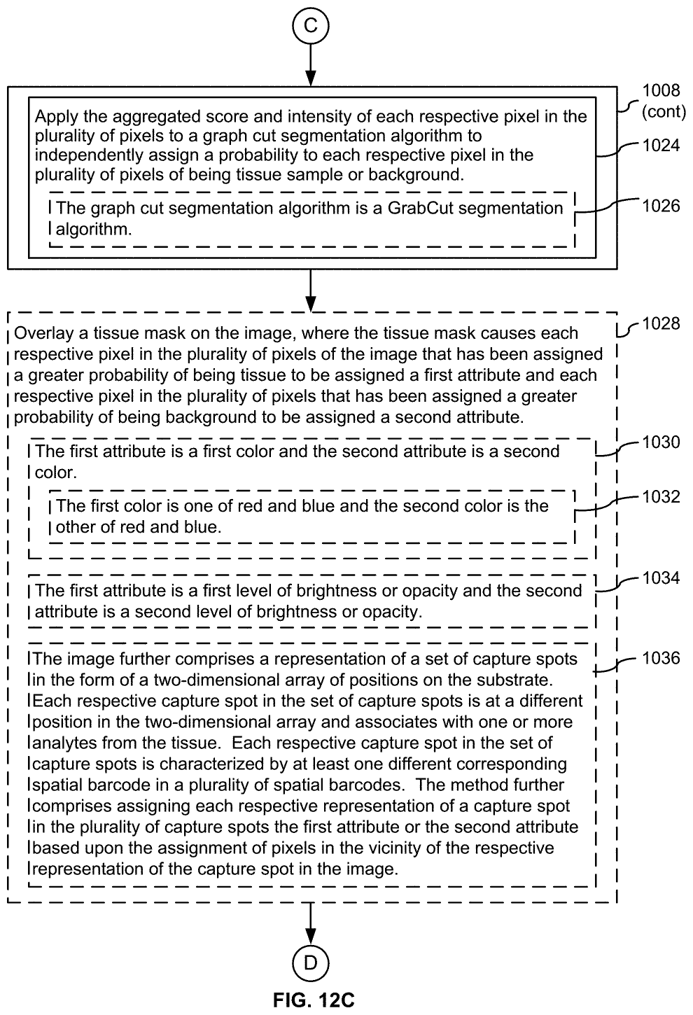

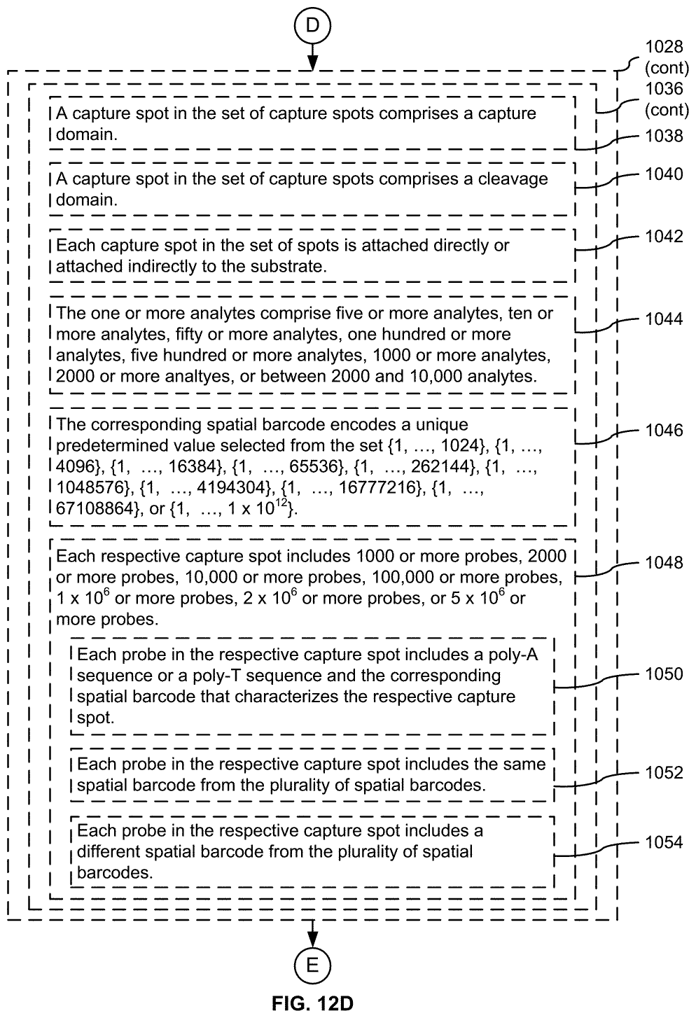

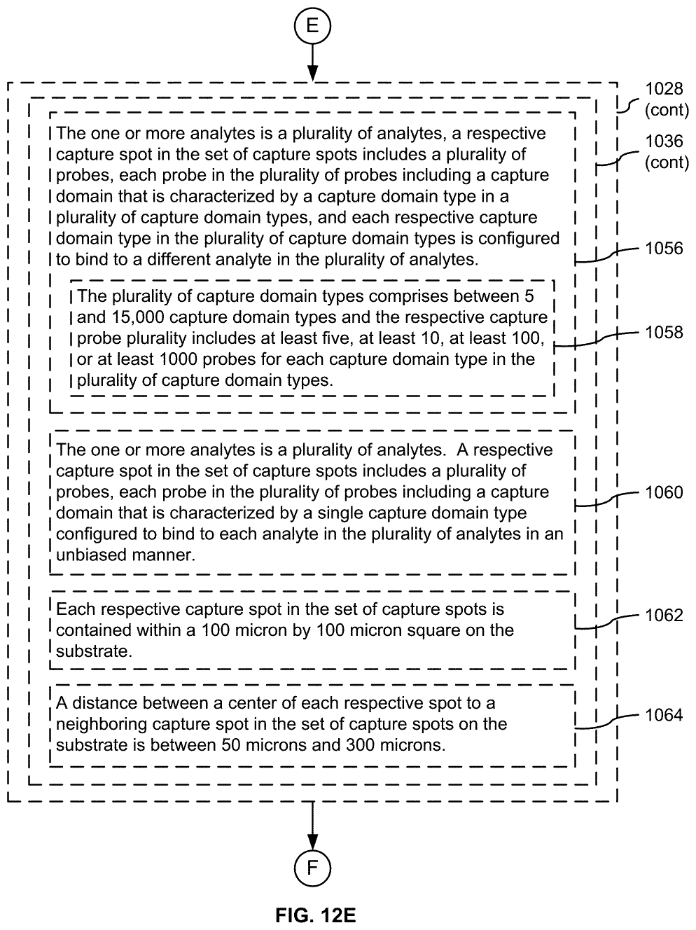

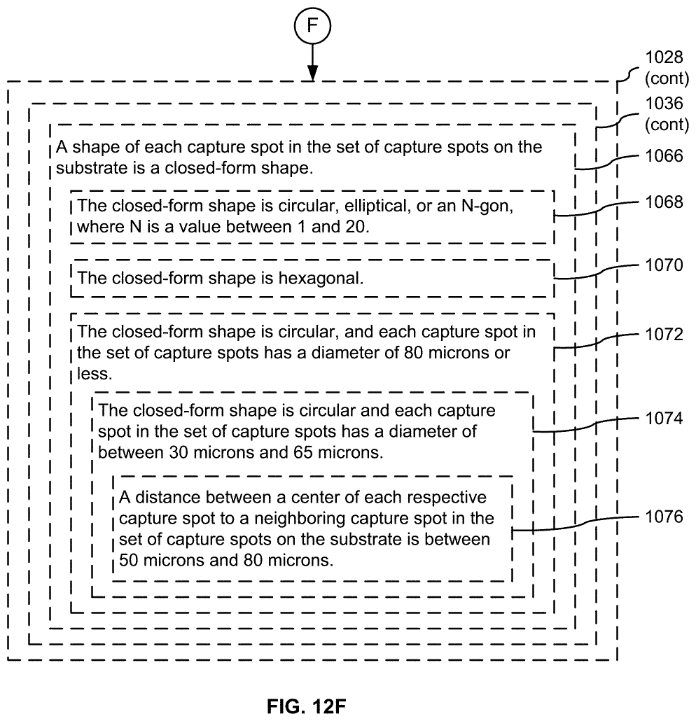

FIG. 12 is a flow chart 1000 illustrating a method for binary tissue classification 1002 in accordance with the present disclosure. In some embodiments, the method takes place at a computer system 1100 having one or more processors 1102, and memory 1112 storing one or more programs for execution by the one or more processors 1102.

(i) Substrates for Spatial Analysis

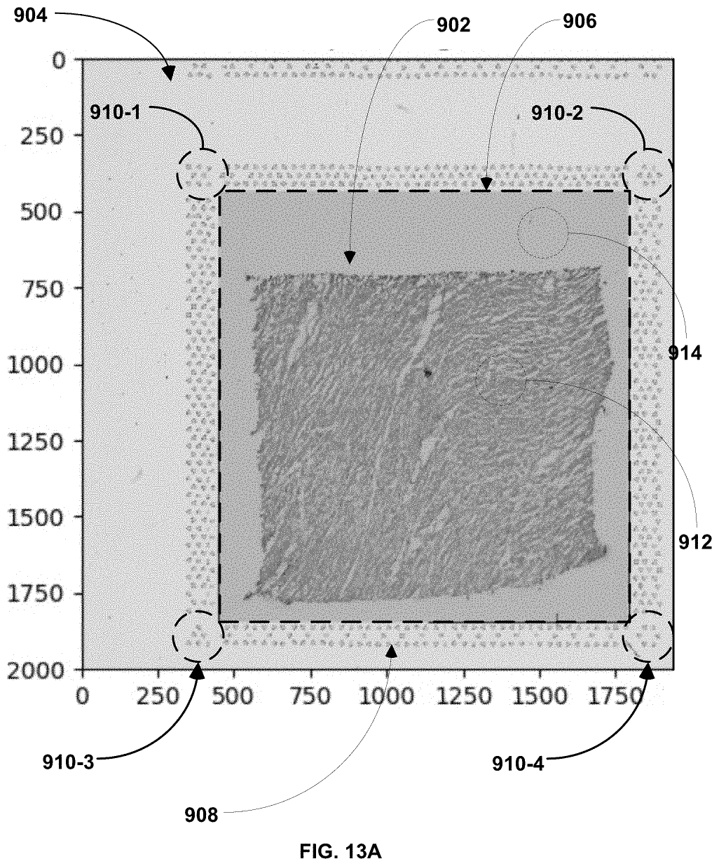

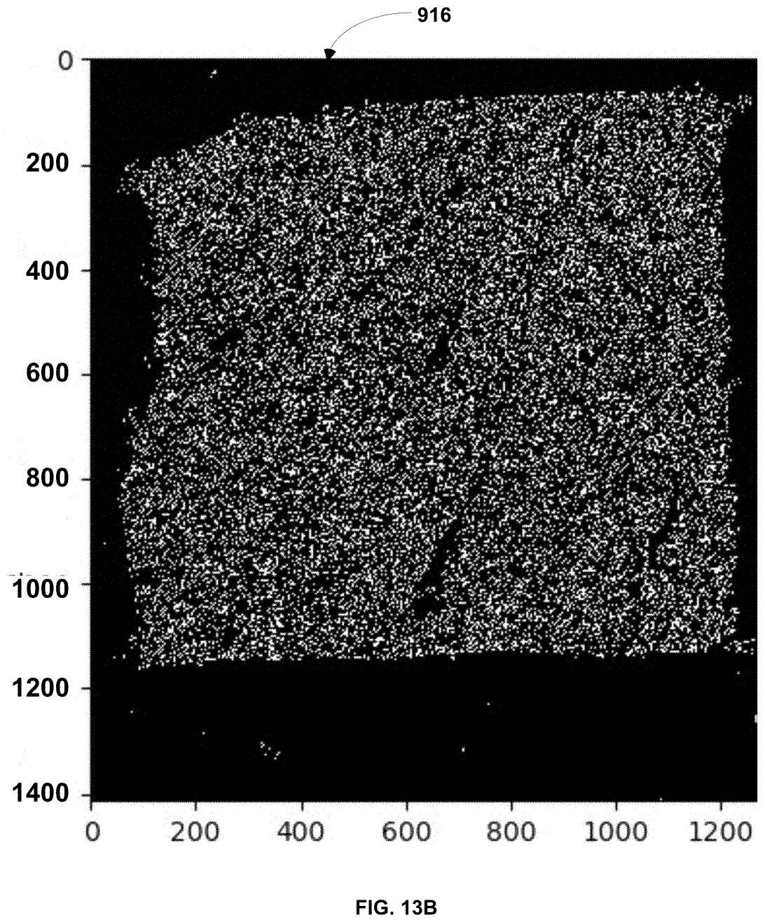

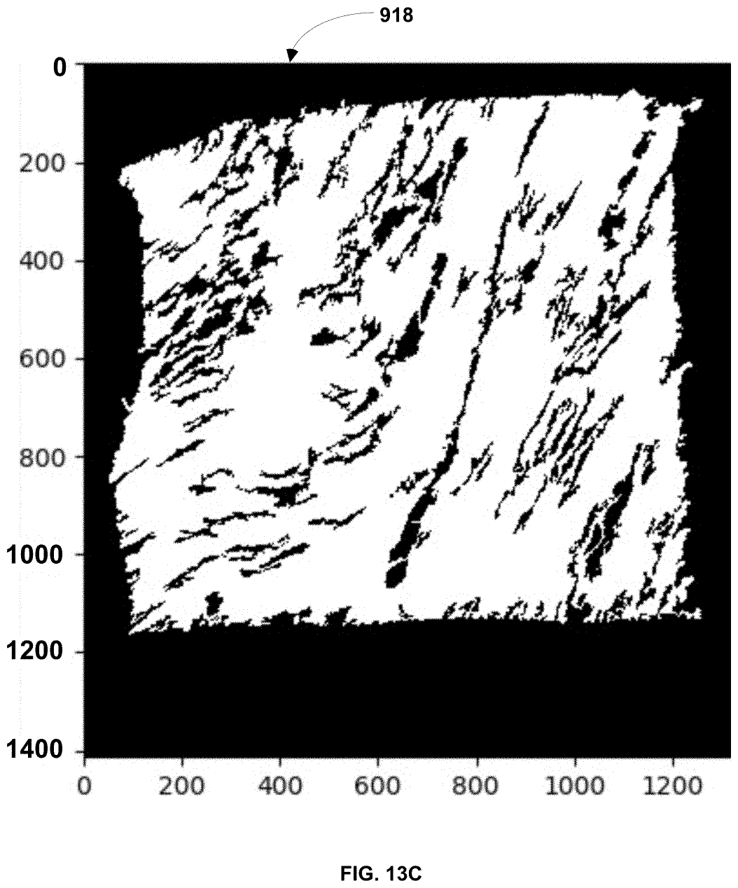

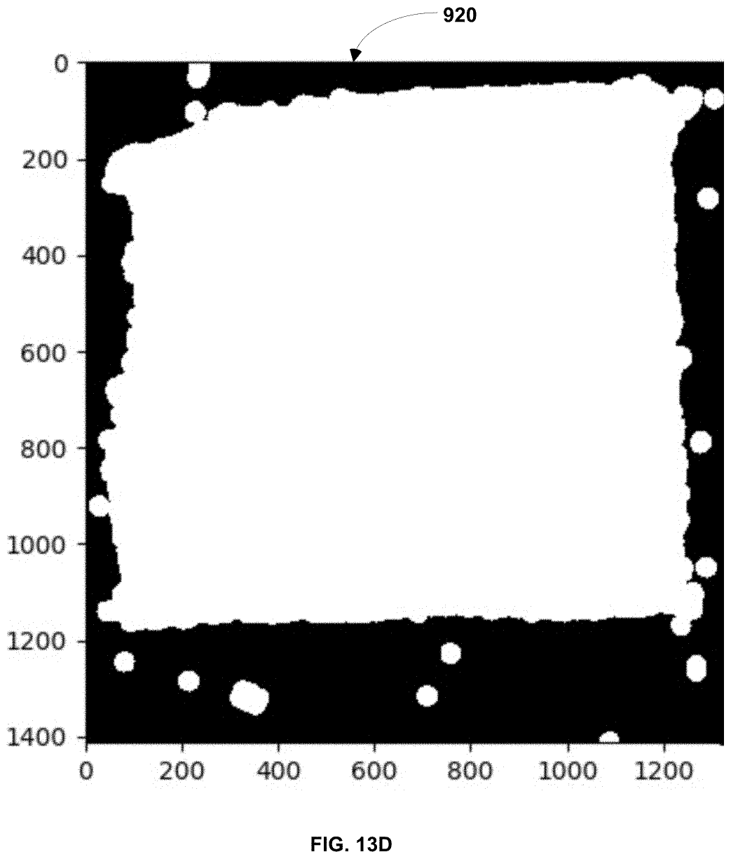

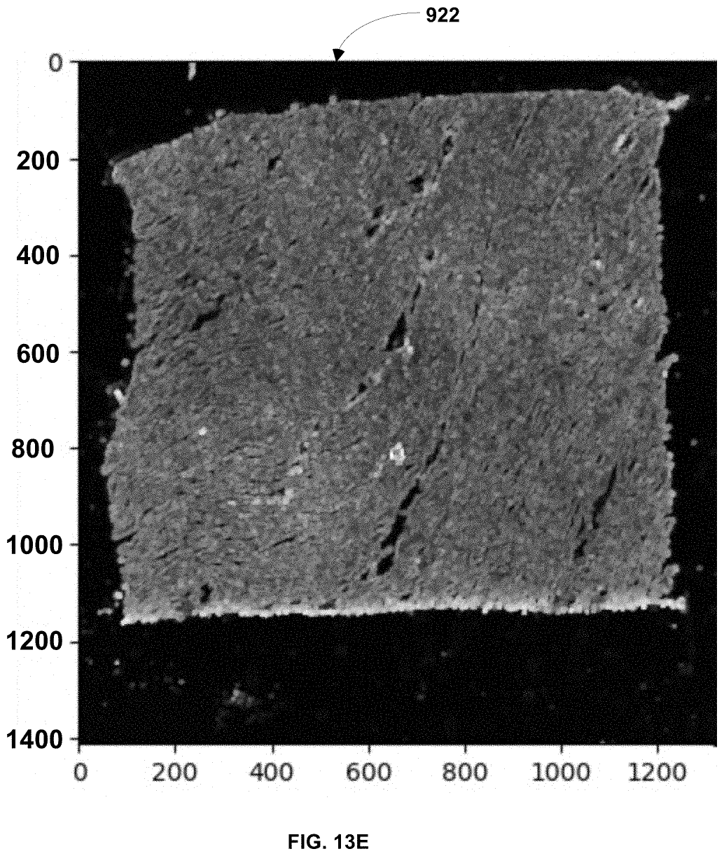

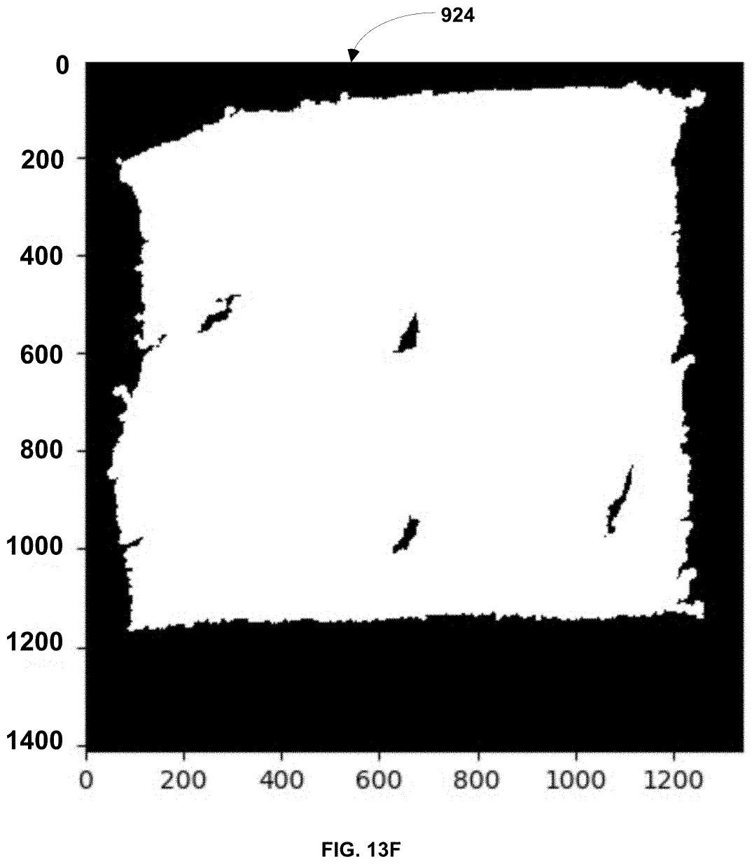

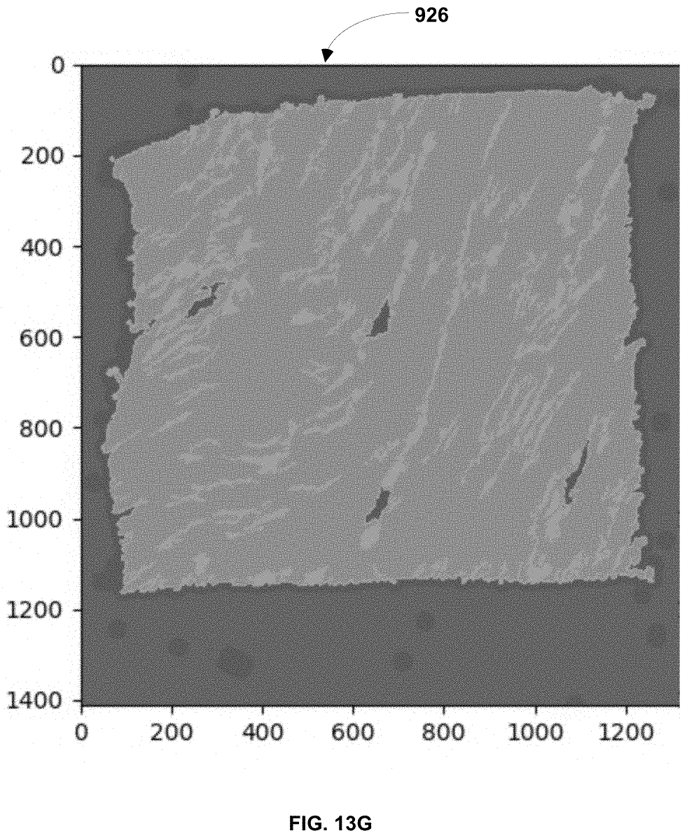

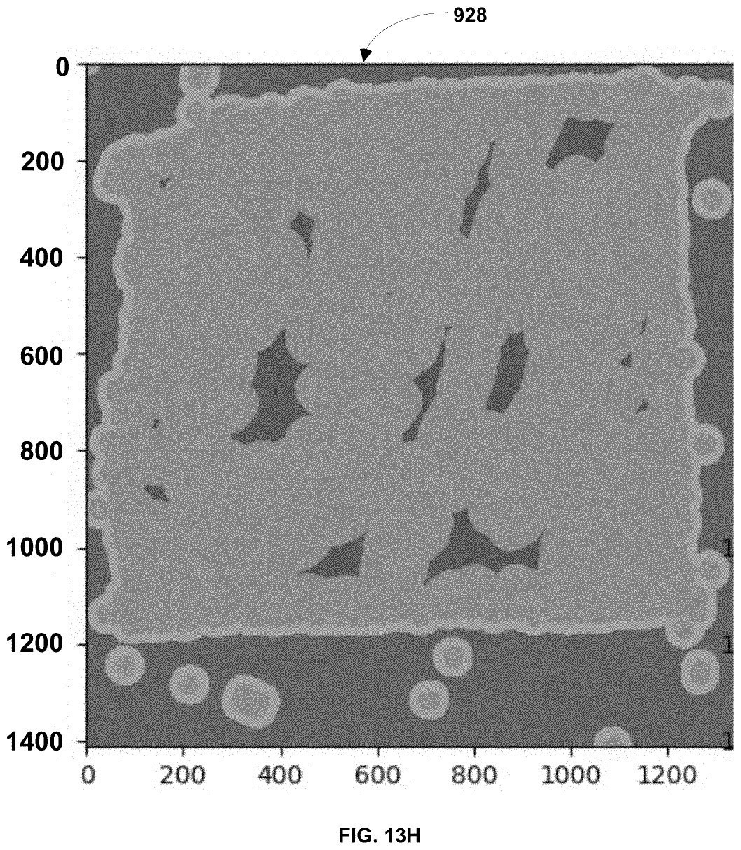

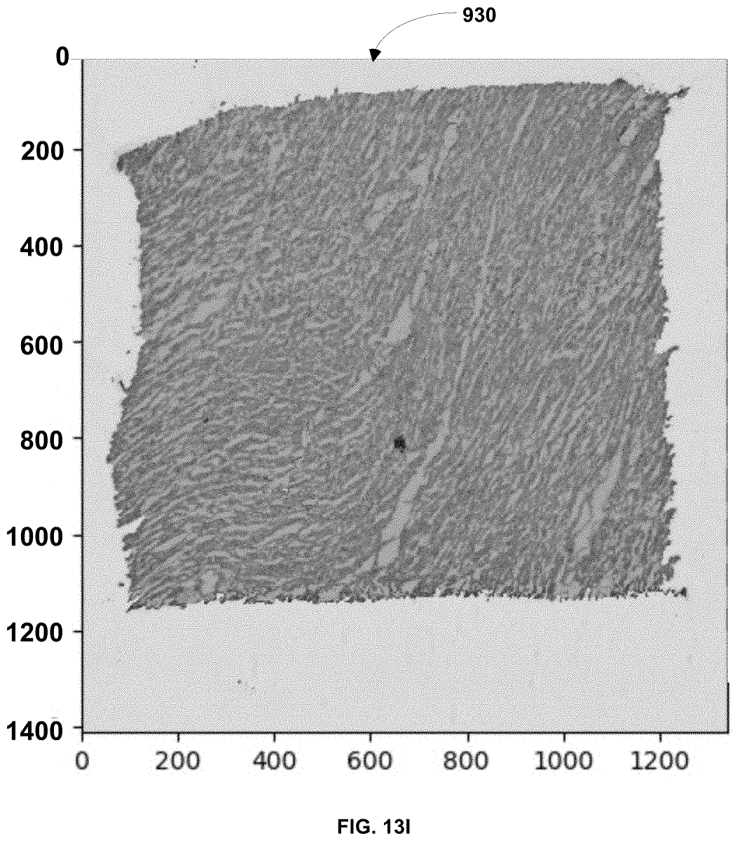

Referring to block 1004, the disclosed method comprises obtaining an image of a sectioned tissue sample overlayed on a substrate. As an illustration, FIG. 13A shows an example of a sectioned tissue sample 902 overlayed on a substrate 904, where the substrate is a slide in accordance with some embodiments. In some embodiments, substrates are used to provide support to a biological sample, particularly, for example, a thin tissue section. In some embodiments, a substrate is a support that allows for positioning of biological samples, analytes, capture spots, and/or capture probes on the substrate. In some embodiments, a substrate can be any suitable support material, including, but not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™ cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate. In some embodiments, a substrate can be printed, patterned, or otherwise modified to comprise capture spots that allow association with analytes upon contacting a biological sample (e.g., a tissue section). Further detailed embodiments of substrate properties, structure, and/or modifications are described above in the Detailed Description (e.g., under II. General Spatial Array-Based Analytical Methodology; (c) Substrate).

As illustrated by the dashed boxed 906 in FIG. 13A, in some embodiments, the substrate comprises a capture area 906. A capture area 906 comprises a plurality of barcoded capture spots for one or more reactions and/or assays. Each such reaction involves spatial analysis of one or more tissue types. The substrate can comprise one or more capture areas 906 for a plurality of reactions and/or assays. What is illustrated in FIG. 13A is a single capture area 906. By contrast, FIG. 9 illustrates a substrate that has six capture areas (906-1, 906-2, 906-3, 906-4, 906-5, and 906-6).

In some embodiments, the substrate is a spatial gene expression slide (e.g., Visium) comprising four capture areas 906, each capture area having the dimensions 6.5 mm×6.5 mm, such that the slide comprises a capacity for four reactions and up to four tissue types. In some such embodiments, each capture area 906 comprises 5,000 barcoded capture spots, where each capture spot is 55 μm in diameter and the distance between the centers of two respective capture spots is 100 μm. Further specific embodiments of capture spots are detailed below in the present disclosure.

(ii) Tissue Samples for Spatial Analysis

Referring again to block 1004, the sectioned tissue sample is obtained from a subject. As defined above, in some embodiments, a subject is a mammal such as a rodent, mouse, rat, rabbit, guinea pig, ungulate, horse, sheep, pig, goat, cow, cat, dog, primate (e.g., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum. These examples are non-limiting and do not preclude substitution of any alternative subjects that will occur to one skilled in the art.

In some embodiments, tissue samples are obtained from any tissue and/or organ derived from any subject, including but not limited to those subjected listed above. In some embodiments, a tissue sample is obtained from, e.g., heart, kidney, ovary, breast, lymph node, adipose, brain, small intestine, stomach, liver, quadriceps, lung, testes, thyroid, eyes, tongue, large intestine, spleen, and/or mammary gland, skin, muscle, diaphragm, pancreas, bladder, prostate, among others. Tissue samples can be obtained from healthy or unhealthy tissue (e.g., inflamed, tumor, carcinoma, or other). Additional examples of tissue samples are shown in Table 1.

TABLE 1
Examples of tissue samples

| Organism | Tissue | Healthy/Diseased |
|---|---|---|
| Human | Brain | Cerebrum Glioblastoma Multiforme |
| Human | Breast | Healthy |
| Human | Breast | Invasive Ductal Carcinoma |
| Human | Breast | Invasive Lobular Carcinoma |
| Human | Heart | Healthy |
| Human | Kidney | Healthy |
| Human | Kidney | Nephritis |
| Human | Large Intestine | Colorectal Cancer |
| Human | Lung | Papillary Carcinoma |
| Human | Lymph Node | Healthy |
| Human | Lymph Node | Inflamed |
| Human | Ovaries | Tumor |
| Human | Spleen | Inflamed |
| Mouse | Brain | Healthy |
| Mouse | Eyes | Healthy |
| Mouse | Heart | Healthy |
| Mouse | Kidney | Healthy |
| Mouse | Large Intestine | Healthy |
| Mouse | Liver | Healthy |
| Mouse | Lungs | Healthy |
| Mouse | Ovary | Healthy |
| Mouse | Quadriceps | Healthy |
| Mouse | Small Intestine | Healthy |
| Mouse | Spleen | Healthy |
| Mouse | Stomach | Healthy |
| Mouse | Testes | Healthy |
| Mouse | Thyroid | Healthy |
| Mouse | Tongue | Healthy |
| Rat | Brain | Healthy |
| Rat | Heart | Healthy |
| Rat | Kidney | Healthy |
| Mouse | Tongue | Healthy |
| Rat | Brain | Healthy |
| Rat | Heart | Healthy |
| Rat | Kidney | Healthy |

In some embodiments, the sectioned tissue is prepared by tissue sectioning, as described above in the Detailed Description (e.g., under I. Introduction; (d) Biological Samples; (ii) Preparation of Biological Samples; (1) Tissue Sectioning). Briefly, in some embodiments, thin sections of tissue are prepared from a biological sample (e.g., using a mechanical cutting apparatus such as a vibrating blade microtome, or by applying a touch imprint of a biological sample to a suitable substrate material). In some embodiments, a biological sample is frozen, fixed and/or cross-linked, or encased in a matrix (e.g., a resin or paraffin block) prior to sectioning to preserve the integrity of the biological sample during sectioning. Further implementations of biological sample preparation are provided above in the Detailed Description (e.g., under I. Introduction; (d) Biological Samples; (ii) Preparation of Biological Samples; (2) Freezing, (3) Formalin Fixation and Paraffin Embedding, (4) Fixation, and (5) Embedding). As an example, referring to FIG. 3, preparation of a biological sample using tissue sectioning comprises a first step 301 of an exemplary workflow for spatial analysis.

Referring to block 1006 of FIG. 12A, in some embodiments, the sectioned tissue sample has a depth of 100 microns or less. Further embodiments of sectioned tissue samples are provided above in the Detailed Description (e.g., under I. Introduction; (d) Biological Samples; (ii) Preparation of Biological Samples; (1) Tissue Sectioning). In some embodiments, a tissue section is a similar size and shape to the substrate on which it is overlayed. In some embodiments, a tissue section is a different size and shape from the substrate on which it is overlayed. In some embodiments, a tissue section overlays all or a portion of the substrate. For example, FIG. 13A illustrates a tissue section with dimensions roughly comparable to the substrate, such that a large proportion of the substrate is in contact with the tissue section.

In some embodiments, a tissue section overlayed on a substrate is a single section. In some alternative embodiments, multiple tissue sections are overlayed on a substrate. In some such embodiments, a single capture area on a substrate can contain multiple tissue sections, where each tissue section is obtained from either the same biological sample and/or subject or from different biological samples and/or subjects. In some embodiments, a tissue section is a single tissue section that comprises one or more regions where no cells are present (e.g., holes, tears, or gaps in the tissue). Thus, in some embodiments such as the above, an image of a tissue section overlayed on a substrate can contain regions where tissue is present and regions where tissue is not present.

(iii) Spatial Substrate Image Acquisition

Referring again to block 1004 of FIG. 12A and with further reference to FIG. 11, the image is obtained as a plurality of pixels 1122 in electronic form. As an example, referring to FIG. 3, imaging of a tissue sample and/or an array on a substrate comprises a second step 302 of an exemplary workflow for spatial analysis. An image can be obtained in any electronic image file format, including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CD5, CPT, PSD, PSP, XCF, PDN, CGM, SVG, PostScript, PCT, WMF, EMF, SWF, XAML, and/or RAW.

In some embodiments, the image is obtained in any electronic color mode, including but not limited to grayscale, bitmap, indexed, RGB, CMYK, HSV, lab color, duotone, and/or multichannel. In some embodiments, the image is manipulated (e.g., stitched, compressed and/or flattened). In some embodiments, the image is represented as an array (e.g., matrix) comprising a plurality of pixels, such that the location of each respective pixel in the plurality of pixels in the array (e.g., matrix) corresponds to its original location in the image. In some embodiments, the image is represented as a vector comprising a plurality of pixels, such that each respective pixel in the plurality of pixels in the vector comprises spatial information corresponding to its original location in the image.

Referring again to block 1004 of FIG. 12A and with further reference to FIG. 13A and FIG. 11, the image includes a plurality of fiducial markers 908 on an outer boundary of the substrate. Fiducial markers are described in further detail in the Detailed Description above (e.g., at II. General Spatial Array-Based Analytical Methodology; (c) Substrate and (e) Analyte Capture; (v) Region of Interest). Briefly, in some embodiments, fiducial markers are included on the substrate 904 as one or more markings on the surface of the substrate. In some embodiments, fiducial markers 908 serve as guides for correlating spatial information with the characterization of the analyte of interest. In some embodiments, fiducial markers 908 are prepared on the substrate 904 using any one of the following non-limiting techniques: chrome-deposition on glass, gold nanoparticles, laser-etching, tubewriter-ink, microspheres, Epson 802, HP 65 Black XL, permanent marker, fluorescent oligos, amine iron oxide nanoparticles, amine thulium doped upconversion nanophosphors, and/or amine Cd-based quantum dots. Other techniques for fiducial marker preparation include sand-blasting, printing, depositing, or physical modification of the substrate surface.

In some embodiments, the fiducial markers 908 are non-transiently attached to the outer boundary of the substrate 904 and the sample is overlayed within the boundary of the fiducial markers. In some embodiments, the fiducial markers are transiently attached to the outer boundary of the substrate (e.g., by attachment of an adaptor, a slide holder, and/or a cover slip), however, the fiducial markers may be placed anywhere on the substrate.

FIG. 13A illustrates an image of a tissue overlayed on a substrate 904, where the image includes a plurality of fiducial markers 908, in accordance with some embodiments. The fiducial markers are arranged along the external border of the substrate, surrounding the capture spot array and the tissue overlay. In some such embodiments, the fiducial markers 908 comprise a collection of patterned spots (e.g., patterns 910-1, 910-2, 910-3, 910-4), and the patterned spots 910 indicate specific edges and corners of the capture spot array. For instance, in some embodiments, each corner of the capture spot array has a unique pattern of fiducial markers (e.g., hourglass 910-1, diamond 910-2, pyramid 910-3, and circle 910-4). As such, in some such embodiments, a different pattern 910 of fiducial markers is provided at each corner, allowing the image to be correlated with spatial information using any orientation (e.g., rotated and/or mirror image).

In some embodiments, the image is acquired using transmission light microscopy. In some embodiments, the image is stained prior to imaging using, e.g., fluorescent, radioactive, chemiluminescent, calorimetric, or colorimetric detectable markers. In some embodiments, the image is stained using live/dead stain (e.g., trypan blue). In some embodiments, biological samples are stained as indicated in the Detailed Description above (e.g., at I. Introduction; (d) Biological Samples; (ii) Preparation of Biological Samples; (6) Staining). In some embodiments, the image is acquired using optical microscopy (e.g., bright field, dark field, dispersion staining, phase contrast, differential interference contrast, interference reflection, fluorescence, confocal, single plane illumination, wide-field multiphoton, deconvolution, transmission electron microscopy, and/or scanning electron microscopy). In some embodiments, the image is acquired after staining the tissue section but prior to analyte capture.

Referring to block 1008 and with further reference to FIG. 13A, the method further comprises assigning each respective pixel in the plurality of pixels to a first class or a second class. The first class indicates overlay of the tissue sample 902 on the substrate 904 and the second class indicates background (meaning no overlay of the tissue sample 902 on the substrate). Thus, for instance, in FIG. 13A, most of the pixels within example region 912 should be assigned the first class and the pixels in example region 914 should be assigned the second class. In some embodiments, the assigning of each respective pixel as tissue (first class) or background (second class) provides information as to the regions of interest, such that any subsequent spatial analysis of the image can be accurately performed using capture spots and/or analytes that correspond to tissue rather than to background. For example, in some instances, obtained images include imaging artifacts including but not limited to debris, background staining, holes or gaps in the tissue section, and/or air bubbles (e.g., under a cover slip and/or under the tissue section preventing the tissue section from contacting the capture array). Then, in some such instances, the ability to distinguish pixels corresponding to tissue from pixels corresponding to background in the obtained image improves the resolution of spatial analysis, e.g., by removing background signals that can impact or obscure downstream analysis, thus limiting the analysis of the plurality of capture probes and/or analytes to a subset of capture probes and/or analytes that correspond to a region of interest (e.g., tissue).

In some embodiments, a region of an image that is not classified as tissue is classified as a hole or an object (e.g., debris, hair, crystalline stain particles, and/or air bubbles). In some such embodiments, small holes and/or objects in an image are defined using a threshold size. In some embodiments, the threshold size is the maximum length (e.g., longest side length) of the image divided by two (e.g., in pixels, inches, centimeters, millimeters, and/or arbitrary units), under which any enclosed shape is considered a hole or an object. In some embodiments, the threshold size is the maximum length of the image divided by N, where N is any positive value greater than or equal to 1. In some embodiments, small holes and objects are removed from the image (e.g., “filled in”) during the assigning of pixels in the image to the first class or the second class, such that an overall region of the image that corresponds to tissue is represented as a contiguous region, and an overall region of the image that corresponds to background is represented as a contiguous region. In some embodiments, small holes and objects are retained in the image during the assigning of pixels in the image to the first class or the second class, such that the region or regions of the image that correspond to tissue do not include small holes and objects, and the region or regions of the image that correspond to background include small holes and objects.

In some embodiments, the assigning of each respective pixel as tissue or background is performed using an algorithm (e.g., implemented via a programming language including but not limited to Python, R, C, C++, Java, and/or Perl), for instance an algorithm implemented by classification module 1120.

(iv) Defining Bounding Boxes using Fiducial Markers

Referring to block 1010 of FIG. 12A and with further reference to FIG. 13A, the assignment of each respective pixel 1122 in the plurality of pixels to a first class or a second class comprises using the plurality of fiducial markers 908 to define a bounding box within the image. In some embodiments, the bounding box 906 has a thickness of one or more pixels. In some embodiments, the bounding box 906 has a shape that is the same shape or a different shape as the original image (e.g., a rectangle, square, circle, oblong shape, or a polygon). In some embodiments, the bounding box 906 has a color (e.g., blue) or is monochromatic (e.g., white, black, gray).

In some embodiments, the bounding box 906 is defined in the same location as (e.g., on top of) the plurality of fiducial markers (e.g., the fiducial frame). In some embodiments, the bounding 906 box is defined within or inside the boundary of the fiducial frame. In some such embodiments, the bounding box 906 is defined as a threshold distance inside of the boundary of the fiducial frame (e.g., one or more pixels, or more than 10, more than 20, more than 30, more than 40, more than 50, or more than 100 pixels inside the fiducial frame). In some embodiments, the bounding box 906 is defined via user input (e.g., a drawn box around the area of interest). In some embodiments, the bounding box 906 is defined using multiple fiducial markers located on at least two opposing corners of the fiducial frame.

In some embodiments, the bounding box 906 is defined using fiducial markers present on the substrate 904 prior to obtaining the image. In some embodiments, the bounding box 906 is defined using fiducial markers 908 added to the image after obtaining the image (e.g., via user input or by one or more heuristic functions). In some embodiments, fiducial alignment is performed to align the obtained image with a pre-defined spatial template using the plurality of fiducial markers as a guide. In some such embodiments, the plurality of fiducial markers in the obtained image are aligned to a corresponding plurality of fiducial markers in the spatial template. In some embodiments, the spatial template comprises additional elements with known locations in the spatial template (e.g., capture spots with known locations relative to the fiducial markers). In some embodiments, the fiducial alignment is performed prior to defining the bounding box (e.g., prior to the assigning of each pixel to the first class or the second class). In some embodiments, fiducial alignment is not performed prior to the defining of the bounding box.

In some embodiments, the bounding box is defined by the edges of the obtained image (e.g., the dimensions of the image) and/or by the field of view (e.g., scope) of the microscope used for obtaining the image. In some embodiments, the bounding box is defined as the adjacent edges at the boundary of the obtained image. In some embodiments, the bounding box is defined as a threshold distance inside the boundary of the obtained image (e.g., one or more pixels inside the boundary of the image). In some embodiments, the bounding box is defined as a set of coordinates (e.g., x-y coordinates) corresponding to each of four corners of the bounding box (e.g., [0+set distance, 0+set distance], [W_{image}−set distance, 0+set distance], [0+set distance, H_{image}−set distance], [W_{image}−set distance, H_{image}−set distance], where W_{image} and H_{image} are the width and height dimensions of the obtained image, respectively, and set distance is a threshold distance inside the boundary of the obtained image). In some embodiments, the threshold distance is pre-defined (e.g., via default and/or user input) or determined heuristically.

In some embodiments, the bounding box is axis-aligned. In some embodiments, the bounding box is centered on the center of the obtained image and/or centered on the center of the region enclosed by the fiducial markers. In some embodiments, the bounding box is not axis-aligned and/or is not centered on either the center of the obtained image or the region enclosed by the fiducial markers. In some embodiments, the threshold distance between each edge of the bounding box and the respective edges of the obtained image and/or the fiducial frame is the same for each respective edge. In some embodiments, the distance between each edge of the bounding box and the respective edges of the obtained image and/or the fiducial frame is different for one or more edges. In some embodiments, the bounding box is rotated on the obtained image to achieve a different alignment of the bounding box against the obtained image.

In some embodiments, no bounding box is defined and the assigning of each respective pixel in the plurality of pixels to a first class or a second class occurs using the obtained image in its entirety. In some such embodiments, a bounding box is defined as “none”.

Referring to block 1012, the assignment of each respective pixel in the plurality of pixels to a first class or a second class further comprises removing respective pixels falling outside the bounding box 906 from the plurality of pixels. Thus, in some embodiments, the method for binary tissue classification only considers pixels inside the bounding box 906. In some embodiments, the removing of pixels falling outside the bounding box 906 is performed by creating a new image from the obtained image, comprising only the respective pixels from the obtained image that fall within the bounding box. In some embodiments, the bounding box is defined as being inside the fiducial frame and the removing of the pixels from the plurality of pixels (e.g., to form image 916) includes removing the fiducial markers from the obtained image. In some embodiments, no bounding box is defined and no pixels are removed from the plurality of pixels.

(v) Application of Heuristic Classifiers to a Tissue Section Image

Referring to block 1014, the assignment of each respective pixel in the plurality of pixels to a first class or a second class further comprises running, after the removing in block 1012, a plurality of heuristic classifiers on the plurality of pixels in grey-scale space. For each respective pixel 1122 in the plurality of pixels, each respective heuristic classifier in the plurality of heuristic classifiers casts a vote 1136 for the respective pixel 1122 between the first class and the second class. Because of this, each pixel 1122 has a series of votes (e.g., 1136-1-1, . . . , 1136-1-N), one from each heuristic classifier. By summing the votes made for a given pixel, an aggregated score 1134 is formed for the given pixel. Thus, a corresponding aggregated score 1134 is formed for each respective pixel 1122 in the plurality of pixels from the individual heuristic classifier votes. In some embodiments, the corresponding aggregated score for each respective pixel is used to convert the aggregated score into a class in a set of classes. In some embodiments, this set of classes comprises obvious first class, likely first class, likely second class, and obvious second class.

In some embodiments, a pixel 1122 comprises one or more pixel values (e.g., intensity value 1124). In some embodiments, each respective pixel in the plurality of pixels comprises one pixel intensity value 1124, such that the plurality of pixels represents a single-channel image comprising a one-dimensional integer vector comprising the respective pixel values for each respective pixel. For example, an 8-bit single-channel image (e.g., grey-scale) can comprise 2⁸ or 256 different pixel values (e.g., 0-255). In some embodiments, each respective pixel 1122 in the plurality of pixels of an image comprises a plurality of pixel values, such that the plurality of pixels represents a multi-channel image comprising a multi-dimensional integer vector, where each vector element represents a plurality of pixel values for each respective pixel. For example, a 24-bit 3-channel image (e.g., RGB color) can comprise 2²⁴ (e.g., 2⁸3) different pixel values, where each vector element comprises 3 components, each between 0-255. In some embodiments, an n-bit image comprises up to 2ⁿ different pixel values, where n is any positive integer.

In some embodiments, the plurality of pixels is in, or is converted to, grey-scale space by obtaining the image in grey-scale (e.g., a single-channel image), or by obtaining the image in color (e.g., a multi-channel image) and converting the image to grey-scale after the obtaining and prior to the running of the heuristic classifiers. In some embodiments, each respective pixel 1122 in the plurality of pixels in grey-scale space has an integer value between 0 and 255 (e.g., 8-bit unsigned integer value or “uint8”). In some embodiments, the integer value for each respective pixel in the plurality of pixels in grey-scale space is transformed using e.g., addition, subtraction, multiplication, or division by a value N, where N is any real number. For example, in some embodiments, each respective pixel in the plurality of pixels in grey-scale space has an integer value between 0 and 255, and each integer value for each respective pixel is divided by 255, thus providing integer values between 0 and 1. In some embodiments, the plurality of pixels of the image is in grey-scale space and is transformed using contrast enhancement or tone curve alignment. In some embodiments, the running of the plurality of heuristic classifiers on the plurality of pixels comprises rotating, transforming, resizing, or cropping the obtained image in grey-scale space.

In some embodiments, the plurality of heuristic classifiers comprises a core tissue detection function, and the plurality of heuristic classifiers comprises one or more heuristic classifiers. In some embodiments, the core tissue detection function makes initial predictions about the placement of the tissue overlayed on the substrate.

Referring to block 1016, in some embodiments, the plurality of heuristic classifiers comprises a first heuristic classifier that identifies a single intensity threshold that divides the plurality of pixels into the first class and the second class. The first heuristic classifier then casts a vote for each respective pixel in the plurality of pixels for either the first class or the second class. The single intensity threshold represents a minimization of intra-class intensity variance between the first and second class or a maximization of inter-class variance between the first class and the second class.

In some embodiments, the single intensity threshold is determined using Otsu's method, where the first heuristic classifier identifies a threshold that minimizes intra-class variance or equivalently maximizes inter-class variance. In some such embodiments, Otsu's method uses a discriminative analysis that determines an intensity threshold such that binned subsets of pixels in the plurality of pixels are as clearly separated as possible. Each respective pixel in the plurality of pixels is binned or grouped into different classes depending on whether the respective intensity value of the respective pixel falls over or under the intensity threshold. For example, in some embodiments, bins are represented as a histogram, and the intensity threshold is identified such that the histogram can be assumed to have a bimodal distribution (e.g., two peaks) and a clear distinction between peaks (e.g., valley).

In some such embodiments, the plurality of pixels in the obtained image is filtered such that pixels comprising a pixel intensity above the intensity threshold are considered to be foreground and are converted to white (e.g., uint8 value of 1), while pixels comprising a pixel intensity below the intensity threshold are considered to be background and are converted to black (e.g., uint8 value of 0). An example of an outcome of a heuristic classifier using Otsu's method is illustrated in FIG. 13C, which depicts a thresholded image 918 (e.g., a mask or a layer) after conversion of the acquired image, where each pixel 1122 in the plurality of pixels is represented as either a white or a black pixel. Here, Otsu's method is an example of a binarization method using global thresholding. In some embodiments, Otsu's method is robust when the variances of the two classes (e.g., foreground and background) are smaller than the mean variance over the obtained image as a whole.

In some embodiments, the first heuristic classifier uses Otsu's method of global thresholding, and the running of the first heuristic classifier is followed by removal of small holes and objects from the thresholded image (e.g., mask). In some such embodiments, the first heuristic classifier provides a more uniform, binary outcome without small perturbations in the mask. In some embodiments, small holes and objects are not removed from the mask such that small holes and objects can be distinguished from tissue.

In some embodiments, the first heuristic classifier is a binarization method other than Otsu's method. In some such embodiments, the first heuristic classifier is a global thresholding method other than Otsu's method or an optimization-based binarization method. In some such embodiments, a global thresholding method is performed by determining the intensity threshold value manually (e.g., via default or user input). For example, an intensity threshold can be determined at the middle value of the grey-scale range (e.g., 128 between 0-255).

In some embodiments, the intensity threshold value is determined automatically using a histogram of grey-scale pixel values (e.g., using the mode method and/or P-tile method). For example, using the mode method, a histogram of grey-scale pixel values can include a plurality of bins (e.g., up to 256 bins for each possible grey-scale pixel value 0-255), and each respective bin is populated with each respective pixel having the respective grey-scale pixel value. In some embodiments, the plurality of bins has a bimodal distribution and the intensity threshold value is the grey-scale pixel value at which the histogram reaches a minimum (e.g., at the bottom of the valley). Using the P-tile method, each respective bin in a histogram of grey-scale pixel values is populated with each respective pixel having the respective grey-scale pixel value, and a cumulative tally of pixels is calculated for each bin from the highest grey-scale pixel value to the lowest grey-scale pixel value. Given a pre-defined number of pixels P above the intensity threshold value, the threshold value is determined at the bin value at which the cumulative sum of pixels exceed P.

In some embodiments, an intensity threshold value is determined by estimating the level of background noise (e.g., in imaging devices including but not limited to fluorescence microscopy). Background noise can be determined using control samples and/or unstained samples during normalization and pre-processing.

In some embodiments, such as when using optimization-based binarization, the assignment of a respective pixel to one of two classes (e.g., conversion to either black or white) is determined by calculating the relative closeness of the converted pixel value to the original pixel value, as well as the relative closeness of the converted pixel value of the respective pixel to the converted pixel values of neighboring pixels (e.g., using a Markov random field). Optimization-based methods thus comprise a smoothing filter that reduces the appearance of small punctate regions of black and/or white and ensures that local neighborhoods exhibit relatively congruent results after binarization.

Referring to block 1018, in some embodiments, the plurality of heuristic classifiers comprises a second heuristic classifier that identifies local neighborhoods of pixels with the same class identified using the first heuristic method. The second heuristic classifier applies a smoothed measure of maximum difference in intensity between pixels in the local neighborhood. The second heuristic classifier thus casts a vote for each respective pixel in the plurality of pixels for either the first class or the second class.

In some embodiments, the local neighborhood of pixels is represented by a disk comprising a radius of fixed length (e.g., one or more pixels). In some embodiments, the disk is used to determine the local intensity gradient, where the local intensity gradient is determined by subtracting the local minimum pixel intensity value (e.g., from the subset of pixels within the disk) from the local maximum pixel intensity value (e.g., from the subset of pixels within the disk), giving a value for each pixel in the subset of pixels within the disk that is a difference of pixel intensities within the local neighborhood. In some such embodiments, a high local intensity gradient indicates tissue, while a low local intensity gradient indicates background.

FIG. 13E illustrates a mask 922 of an obtained image where each pixel 1122 in the plurality of pixels in the obtained image is converted to a grey-scale value that is a difference in local intensity values. Unlike the global thresholding methods (e.g., Otsu's method) described above, local intensity gradients are a measure of granularity rather than intensity. For example, whereas global thresholding methods distinguish subsets of pixels that are relatively “light” from subsets of pixels that are relatively “dark,” local intensity gradients distinguish regions with patterns of alternating lightness and darkness (e.g., texture) from regions with relatively constant intensities (e.g., smoothness). Local intensity gradient methods are therefore robust in some instances where images comprise textured tissue and moderate resolution, and/or where global thresholding techniques fail to distinguish between classes due to various limitations. These include, in some embodiments, small foreground size compared to background size, small mean difference between foreground and background intensities, high intra-class variance (e.g., inconsistent exposure or high contrast within foreground and/or background regions), and/or background noise (e.g., due to punctate staining, punctate fluorescence, or other intensely pigmented areas resulting from overstaining, overexposure, dye residue and/or debris).

In some embodiments, the first or second heuristic classifier comprises a smoothing method to minimize or reduce noise between respective pixels 1122 in a local neighborhood by filtering for differences in pixel intensity values. In some embodiments, smoothing is performed in a plurality of pixels in grey-scale space. In some embodiments, applicable smoothing methods include, but are not limited to, blurring filters, median filters, and/or bilateral filters. For example, in some embodiments, a blurring filter minimizes differences within a local neighborhood by replacing the pixel intensity values 1124 at each respective pixel 1122 with the average intensity values of the local neighborhood around the respective pixel 1122. In some embodiments, a median filter utilizes a similar method, but replaces the pixel intensity values 1124 at each respective pixel with the median pixel values of the local neighborhood around the respective pixel 1122. Whereas, in some embodiments, blurring filters and median filters cause image masks to exhibit “fuzzy” edges, in some alternative embodiments, a bilateral filter preserves edges by determining the difference in intensity between pixels 1122 in a local neighborhood and reducing the smoothing effect in regions where a large difference is observed (e.g., at an edge).

Thus, in some embodiments, a second heuristic classifier comprises a local intensity gradient filter for a disk with a fixed-length radius also functions as a smoothing filter for the plurality of pixels 1122 in the obtained image. The size of the local area defines the smoothing, such that increasing the radius of the disk would increasing the smoothing effect, while decreasing the radius of the disk would increase the resolution of the classifier.

In some embodiments, a global thresholding method is further applied to an image mask comprising the outcome of a local intensity gradient filter represented as an array (e.g., a matrix) of grey-scale pixel values. In some such embodiments, the local intensity gradient array is binarized into two classes using Otsu's method, such that each pixel in the plurality of pixels is converted to a white or a black pixel (e.g., having pixel value of 1 or 0, respectively), representing foreground or background, respectively. FIG. 13F illustrates an example 924 of the characterization of pixels into the first and second class using Otsu's method applied to a local intensity gradient filter from an obtained image, such that binarization is applied to regions of high and low granularity rather than regions of high and low pixel intensity. This provides an alternative method for classifying foreground and background regions over global thresholding methods.

In some embodiments, binarized local intensity gradients can be further processed by removing small holes and objects, as described previously. In some embodiments, small holes and objects are not removed from binarized local intensity gradient arrays. In some embodiments, a local intensity gradient filter is applied to a thresholded image generated using Otsu's method. In some embodiments, a plurality of heuristic classifiers is applied sequentially to an obtained image such that a second heuristic classifier is applied to a mask resulting from a first heuristic classifier, and a third heuristic classifier is applied to a mask resulting from the second heuristic classifier. In some alternative embodiments, a plurality of heuristic classifiers is applied to an obtained image such that each respective heuristic classifier is independently applied to the obtained image and the independent results are combined. In some embodiments, a plurality of heuristic classifiers is applied to an obtained image using a combination of sequentially and independently applied heuristic classifiers.

In some embodiments, a second heuristic classifier is a two-dimensional Otsu's method, which, in some instances, provides better image segmentation for images with high background noise. In the two-dimensional Otsu's method, the grey-scale intensity value of a respective pixel 1122 is compared with the average intensity of a local neighborhood. Rather than determining a global intensity threshold over the entire image, an average intensity value is calculated for a local neighborhood within a fixed distance radius around the respective pixel 1122, and each pair of intensity values (e.g., a value averaged over the local neighborhood and a value for the respective pixel 1122) are binned into a discrete number of bins. The number of instances of each pair of average intensity values for the local neighborhood and for the respective pixel 1122, divided by the number of pixels in the plurality of pixels, determines a joint probability mass function in a 2-dimensional histogram. In some embodiments, the local neighborhood is defined by a disk comprising a radius of fixed length (e.g., one or more pixels 1122).

Referring to block 1020, in some embodiments, the plurality of heuristic classifiers comprises a third heuristic classifier that performs edge detection on the plurality of pixels to form a plurality of edges in the image and morphologically closes the plurality of edges to form a plurality of morphologically closed regions in the image. The third heuristic classifier then assigns pixels 1122 in the morphologically closed regions to the first class and pixels 1122 outside the morphologically closed regions to the second class, thereby causing the third heuristic classifier to cast a vote for each respective pixel 1122 in the plurality of pixels for either the first class or the second class.

In some embodiments, a Canny edge detection algorithm is used to detect edges on a grey-scale image. In some such embodiments, edges are identified using a convolution algorithm that identifies the pixel intensity value 1124 for each respective pixel 1122 in a plurality of pixels in an array (e.g., an image or a mask) and compares two or more pixels to an edge detection filter (e.g., a box operator that represents a threshold difference in pixel intensity). An edge is thus defined as a set of pixels with a large difference in pixel intensities. Identification of edges is determined by calculating the first-order or second-order derivatives of neighboring pixel intensity values. In some embodiments, the Canny edge detection algorithm results in a binary image where a particular first assigned color value (e.g., white) is applied to pixels that represent edges whereas pixels that are not part of an edge are assigned a second color value (e.g., black). FIG. 13B illustrates an image mask 916 comprising the output of a Canny edge detection algorithm on an obtained image.

In some embodiments, edge detection is performed using an edge detection filter other than a Canny edge detection algorithm, including but not limited to Laplacian, Canny, Sobel, Canny-Deriche, Log Gabor, and/or Marr-Hildreth. In some embodiments, a smoothing filter is applied prior to applying the edge detection filter to suppress background noise.

In some embodiments, edges in the plurality of edges are closed to form a plurality of morphologically closed regions. In some embodiments, morphological closing is performed on the plurality of pixels in grey-scale space. In some embodiments, morphological closing comprises a dilation followed by an erosion. In some embodiments, the plurality of pixels in the morphologically closed regions are expressed as an array of 1's and 0's, where pixels assigned to a first class are expressed as 1's (e.g., closed regions) and pixels assigned to a second class are expressed as 0's (e.g., unclosed regions). In some embodiments, the array of 1's and 0's comprise a mask of the image that stores the results of the edge detection and subsequent morphological closing. FIG. 13D illustrates an image mask 920 in which closed regions are formed by morphologically closing a plurality of edges identified using a Canny edge detection algorithm, as pictured in FIG. 13B. Closed and unclosed regions comprise a plurality of pixels that are expressed as pixel values 1 and 0, respectively, and are visualized as, for example, white and black pixels, respectively.

In some embodiments, the plurality of heuristic classifiers comprises one or more heuristic classifier described above or any combination thereof. These embodiments are non-limiting and do not preclude substitution of any alternative heuristic classifiers for image manipulation, transformation, binarization, filtration, and segmentation as will be apparent to one skilled in the art.

Referring to block 1022, in some embodiments, the plurality of heuristic classifiers consists of a first, second, and third heuristic classier, each respective pixel 1122 assigned by each of the heuristic classifiers in the plurality of classifiers to the second class is labelled as obvious second class, and each respective pixel 1122 assigned by each of the plurality of heuristic classifiers as the first class is labelled as obvious first class. For example, in some such embodiments, the plurality of heuristic classifiers consists of a first, second and third heuristic classifier, and each respective classifier casts a vote 1136 for each respective pixel 1122 in the plurality of pixels for either the first class or the second class (e.g., tissue or background, respectively). In some such embodiments, the plurality of votes is aggregated and the aggregate score 1134 determines whether the respective pixel 1122 is classified as obvious first class, likely first class, likely second class, or obvious second class. In some embodiments, for each respective pixel 1122 in a plurality of pixels in grey-scale space, each respective vote 1136 for the first class (e.g., foreground and/or tissue) is 1, and each respective vote 1136 for the second class (e.g., background) is 0. Thus, for example, an aggregate score 1134 of 0 indicates three votes for background, an aggregate score of 1 indicates one vote for tissue and two votes for background, an aggregate score of 2 indicates two votes for tissue and one vote for background, and an aggregate score of 3 indicates three votes for tissue. FIG. 13G illustrates an image mask 926 representing a sum of a plurality of heuristic classifiers, where each aggregate score 1134 is represented as one of a set of four unique classes comprising 0, 1, 2, and 3. In some embodiments, small holes and objects are detected using the image mask of the aggregated scores using a morphological detection algorithm (e.g., in Python).

In some embodiments, a respective pixel 1122 in the plurality of pixels is classified as obvious first class, likely first class, likely second class, or obvious second class based on the number and/or type of heuristic classifier votes 1136 received. For example, in some embodiments, a respective pixel 1122 that receives three votes 1136 for background is classified as obvious background, and a respective pixel 1122 that receives one vote 1136 for tissue in classified as probable background. In some alternative embodiments, a respective pixel 1122 that receives one vote 1136 for tissue is classified as probable tissue, and a respective pixel 1122 that receives two or more votes 1136 for tissue is classified as obvious tissue.

In some embodiments, a respective pixel 1122 that is classified by at least one heuristic classifier as a hole or object is classified as probable background (e.g., to ensure that that “holes” of non-covered areas surrounded by tissue are initialized with non-“obvious” labels). In some embodiments, a region (a number of pixels in the region) of an obtained image that is classified as obvious tissue based on at least two heuristic classifier votes 1136 is reduced in size (e.g., a border of a detected region is resized inward) by a first fixed-length margin. In some embodiments, the first fixed-length margin is one or more pixels 1122. In some embodiments, the first fixed-length margin is a percentage of a length of a side of the obtained image. In some embodiments, the first fixed-length margin is between 0.5% and 10% of the length of the longest side of the obtained image. In some embodiments, a region of an obtained image that is classified as obvious tissue based on at least three heuristic classifier votes is reduced in size by a second fixed-length margin that is smaller than the first fixed-length margin. In some embodiments, the second fixed-length margin has a length that is one-half the length of the first fixed-length margin.

In some embodiments, a respective heuristic classifier is given priority and/or greater weight in the aggregated score. For example, in some embodiments, the first heuristic classifier is global thresholding by Otsu's method. In some such embodiments, a region of an obtained image that is classified as tissue by at least one other heuristic classifier and is not classified as a hole or an object is nevertheless classified as probable background if it is not classified as tissue by the first heuristic classifier (e.g., Otsu's method). In some embodiments, a respective heuristic classifier in the plurality of heuristic classifiers is given priority and/or greater weight in the aggregated score depending on the order in which the respective heuristic classifier is applied (e.g., first, second, or third), or depending on the type of classifier applied (e.g., Otsu's method). In some embodiments, each respective heuristic classifier in the plurality of heuristic classifiers is given equal weight in the aggregated score.

In some embodiments, the aggregated score 1134 formed from the plurality of votes 1136 from the plurality of heuristic classifiers is a percentage of votes for a first class out of a total number of votes. In some such embodiments, each class in the set of classes comprising obvious first class, likely first class, likely second class, and obvious second class corresponds to a percentage of votes for a first class out of the total number of votes. In some alternative embodiments, each class in the set of classes comprising obvious first class, likely first class, likely second class, and obvious second class corresponds to a number of votes above a threshold number of votes out of the plurality of votes from the plurality of heuristic classifiers. In some embodiments, a specific “truth table” is pre-defined (e.g., via default or user input), giving the respective class assignments for each respective aggregated score.

In some embodiments, a respective pixel 1122 that is not assigned a class by any prior method is classified as probable background.

In some embodiments, the classifying of each respective pixel 1122 in the plurality of pixels to a class in a set of classes comprising obvious first class, likely first class, likely second class, and obvious second class based on the aggregated score generates a separate array (e.g., image mask), where each pixel 1122 in the array comprises a respective separate value or attribute corresponding to the assigned class in the set of classes. FIG. 13H illustrates an image mask 928 where each pixel 1122 is represented by an attribute corresponding to obvious first class, likely first class, likely second class, and obvious second class. Notably, the image masks in FIG. 13G and FIG. 13H differ in that the image mask 926 in FIG. 13G represents a raw aggregate of the plurality of votes from the plurality of heuristic classifiers, whereas the image mask 928 in FIG. 13H represents the subsequent classification of each respective pixel 1122 based on the aggregated score 1134. As described above, in some embodiments, classification of a respective pixel 1122 based on the aggregated score 1134 is not dependent solely on the raw sum of the plurality of votes 1136 but is, in some instances, dependent on the order and/or importance of a respective heuristic classifier in the plurality of heuristic classifiers. Thus, the image masks depicted in FIG. 13G and FIG. 13H are similar but not identical, in accordance with some embodiments.

In some embodiments an image mask is generated for quality control purposes (e.g., to provide visual confirmation of classification outcomes to a user or practitioner). In some embodiments, an image mask is generated in grey-scale or in multispectral color (e.g., RGB, 24-bit RGB, and/or float64-bit RGB). In some embodiments, the image mask is re-embedded on the original obtained image for comparison and/or quality control purposes. In some embodiments, an image mask generated at any stage and/or following any number of one or more heuristic classifiers is re-embedded on the original obtained image, and the re-embedding comprises rotating, resizing, transforming, or overlaying a cropped image mask onto the original obtained image.

In some embodiments, the image mask 928 generated by the classification of each respective pixel 1122 in the plurality of pixels to a class in the set of classes, as depicted in the example of FIG. 13H, is used as markers for downstream image segmentation (e.g., GrabCut markers). In some embodiments, the image mask used for markers for downstream image segmentation is generated prior to applying the plurality of heuristic classifiers to the obtained image and is iteratively constructed and reconstructed based on the aggregated scores for the plurality of heuristic classifiers after applying each respective heuristic classifier in the plurality of heuristic classifiers. Thus, in some such embodiments, a pixel 1122 is in some instances assigned a first classification that is changed to a second classification after the application of subsequent heuristic classifiers.

In some embodiments, the plurality of heuristic classifiers comprises a core tissue detection function that provides initial estimates of the tissue placement, and these estimates are combined into an initialization prediction that is passed to a subsequent segmentation algorithm.

(vi) Image Segmentation

Referring to block 1024, the method for binary tissue classification further comprises applying the aggregated score 1134 and intensity 1124 of each respective pixel 1122 in the plurality of pixels to a graph cut segmentation algorithm to independently assign a probability to each respective pixel in the plurality of pixels of being tissue sample or background.

Graph cut performs segmentation of a monochrome image based on an initial trimap T={T_{B}, T_{U}, T_{F}}, where T_{B} indicates background regions, T_{F} indicates foreground regions, and T_{U} indicates unknown regions. The image is represented as an array z=(z₁, . . . , zₙ, . . . , z_{N}) comprising grey-scale pixel values for a respective pixel n in a plurality of N pixels. As in Bayes matting models, the graph cut segmentation algorithm attempts to compute the alpha values for To given input regions for T_{B} and T_{F}, by creating an alpha-matte that reflects the proportion of foreground and background for each respective pixel in a plurality of pixels as an alpha value between 0 and 1, where 0 indicates background and 1 indicates foreground. In some embodiments, an alpha value is computed by transforming a grey-scale pixel value (e.g., for an 8-bit single-channel pixel value between 0 and 255, the pixel value is divided by 255). Graph cut is an optimization-based binarization technique as described above, which uses polynomial-order computations to achieve robust segmentation even when foreground and background pixel intensities are poorly segregated.

In some embodiments, the trimap is user specified. In some embodiments, the trimap is initialized using the plurality of heuristic classifiers as an initial tissue detection function. In some such embodiments, the set of classes comprising obvious first class, likely first class, likely second class, and obvious second class are provided to the graph cut segmentation algorithm as a trimap comprising T_{F}={obvious first class} (e.g., obvious foreground), T_{B}={obvious second class} (e.g., obvious background), and T_{U}={likely first class, likely second class} (e.g., concatenation of likely foreground and likely background). In some embodiments, the T_{F}={obvious first class, probable first class} (e.g., obvious foreground and probable foreground), T_{B}={obvious second class, probable second class} (e.g., obvious background and probable background), and T_{U} is any unclassified pixels in the plurality of pixels in the obtained image. In some embodiments, the set of classes is provided to the graph cut segmentation algorithm using an alternate trimap that is a combination or substitution of the above implementations that will be apparent to one skilled in the art.

Referring to block 1026, in some embodiments, the graph cut segmentation algorithm is a GrabCut segmentation algorithm. The GrabCut segmentation algorithm is based on a graph cut segmentation algorithm, but includes an iterative estimation and incomplete labelling function that limits the level of user input required and utilizes a an alpha computation method used for border matting to reduce visible artefacts. Furthermore, GrabCut uses a soft segmentation approach rather than a hard segmentation approach. Unlike graph cut segmentation algorithms, GrabCut uses Gaussian Mixture Models (GMMs) instead of histograms of labelled trimap pixels, where a GMM for a background and a GMM for a foreground are full-covariance Gaussian mixtures with K components. To make the GMM a tractable computation, a unique GMM component is assigned to each pixel in the plurality of pixels from either the background or the foreground model (e.g., 0 or 1).

In some embodiments, the GrabCut segmentation algorithm can operate either on a multi-spectral, multi-channel image (e.g., a 3-channel image) or on a single-channel image. In some embodiments, a grey-scale image is provided to the segmentation algorithm. In some embodiments, a grey-scale image is first converted to a multi-spectral, multi-channel image (e.g., RGB, HSV, CMYK) prior to input into the segmentation algorithm. In some embodiments, a multi-spectral, multi-channel color image is applied directly to the segmentation algorithm.

In some embodiments, the GrabCut segmentation algorithm is applied to the image as a convolution method, such that local neighborhoods are first assigned to a classification (e.g., foreground or background) and assignations are then applied to a larger area. In some embodiments, an image comprising a plurality of pixels is provided to the GrabCut algorithm as a color image, using the initialization labels obtained from the plurality of heuristic classifiers, and the binary classification output of the GrabCut algorithm is used for downstream spatial analysis (e.g., on barcoded capture spots). In some embodiments, the plurality of pixels assigned with a greater probability of tissue or background is used to generate a separate construct (e.g., a matrix, array, list or vector) indicating the positions of tissue and the positions of background in the plurality of pixels. For example, FIG. 13I illustrates an image mask resulting from the GrabCut algorithm for an obtained image FIG. 13A given an input trimap based on GrabCut markers as illustrated in FIG. 13H. The GrabCut segmentation algorithm performs binary identification of tissue and background, which is evident from the clear isolation of the tissue section overlay from the background regions.

In some embodiments, the aggregated score and intensity of each respective pixel in the plurality of pixels is applied to a segmentation algorithm other than a graph cut segmentation algorithm or a GrabCut segmentation algorithm, including but not limited to, Magic Wand, Intelligent Scissors, Bayes Matting, Knockout 2, level sets, binarization, background subtraction, watershed method, region growing, clustering, active contour model (e.g., SNAKES), template matching and recognition-based method, Markov random field. In some embodiments, the aggregated score and intensity of each respective pixel in the plurality of pixels is applied to a feature extraction algorithm (e.g., intuition and/or heuristics, gradient analysis, frequency analysis, histogram analysis, linear projection to a trained low-dimensional subspace, structural representation, and/or comparison with another image). In some embodiments, the aggregated score and intensity of each respective pixel in the plurality of pixels is applied to a pattern classification method including but not limited to nearest neighbor classifiers, discriminant function methods (e.g., Bayesian classifier, linear classifier, piecewise linear classifier, quadratic classifier, support vector machine, multilayer perception/neural network, voting), and/or classifier ensemble methods (e.g., boosting, decision tree/random forest).

(vii) Applications of Tissue Detection for Spatial Analysis

Referring to block 1028, in some embodiments, the method further comprises overlaying a tissue mask on the image, where the tissue mask causes each respective pixel in the plurality of pixels of the image that has been assigned a greater probability of being tissue to be assigned a first attribute and each respective pixel in the plurality of pixels that has been assigned a greater probability of being background to be assigned a second attribute.

In some embodiments, the assigning of a first or a second attribute to a respective pixel requires a threshold value for the respective pixel, such that a pixel value above or below the threshold value is assigned a greater probability of being tissue or a greater probability of being background, respectively (e.g., a pixel value between 0 and 1, or a pixel value between 0 and 255). In some embodiments a greater probability of being tissue or a greater probability of being background is assigned based on the aggregated score corresponding to the class in the set of classes that is obvious first class and/or likely first class, or obvious second class and/or likely second class, respectively. In some embodiments, a greater probability of being tissue or a greater probability of being background is determined using an image segmentation algorithm, which applies a binary classification to each respective pixel in a plurality of pixels in an obtained image.

Referring to block 1030, in some such embodiments, the first attribute is a first color and the second attribute is a second color. Referring to block 1032, in some such embodiments, the first color is one of red and blue and the second color is the other of red and blue. In some embodiments, the first color is any one of a group comprising red, orange, yellow, green, blue, violet, white, black, gray, and/or brown, and the second color is any one of the same group that is a different color than the first color. Referring to block 1034, in some embodiments, the first attribute is a first level of brightness or opacity and the second attribute is a second level of brightness or opacity. In some embodiments, the first and second attributes are any contrasting attributes for a visual representation of binary class (e.g., zeros and ones, colors, contrasting shades and/or pixel intensities, symbols (e.g., X's and O's), and/or patterns (e.g., hatch patterns)).

In some embodiments, attributes are assigned based on both class assignment (e.g., tissue or background) and probability (e.g., obvious or likely). For example, in some embodiments, a respective pixel in a plurality of pixels in an obtained image is assigned a first attribute and a second attribute for a first parameter that indicates whether the respective pixel corresponds to a region of overlay of the tissue sample or a region of background (e.g., a red color and a blue color), and a first attribute and a second attribute for a second parameter that indicates the probability and/or likelihood of the class assignation (e.g., a level of brightness or opacity). Thus, in some such embodiments, a respective pixel comprises a plurality of attributes (e.g., dark red, light red, light blue, dark blue).

In some embodiments, attributes are assigned based on both class assignment (e.g., tissue or background) and pixel intensity. In some embodiments, respective pixel in a plurality of pixels in an obtained image is assigned two or more attributes for a plurality of parameters.

Referring to block 1036 and with further reference to FIG. 9, in some embodiments, the image further comprises a representation of a set of capture spots (e.g., 1202-1, . . . , 1202-4, . . . , 1202-13, . . . , 1202-M) in the form of a two-dimensional array 1138 of positions on the substrate 904. Each respective capture spot 1202 in the set of capture spots is (i) at a different position in the two-dimensional array 1138 and (ii) associates with one or more analytes from the tissue. Each respective capture spot 1202 in the set of capture spots is characterized by at least one different corresponding spatial barcode in a plurality of spatial barcodes. FIG. 10 illustrates one such capture spot 1202.

In some such embodiments, the method further comprises assigning each respective representation of a capture spot 1202 in the plurality of capture spots the first attribute or the second attribute based upon the assignment of pixels in the vicinity of the respective representation of the capture spot in the image. For instance, referring to FIG. 9, capture spots 1202-1, . . . , 1202-4, . . . , 1202-13, . . . , 1202-M would be assigned to background because they fall outside the region sectioned tissue 1204 is overlayed onto.

In some embodiments, the assignment of a first or second attribute to a respective representation of a capture spot 1202 in the plurality of capture spots is represented as a tissue position construct (e.g., a matrix, array, list or vector) indicating the positions of tissue and background respective to the plurality of pixels and/or respective to the plurality of capture spots, thus indicating the subset of pixels corresponding to the subset of capture spots that is overlayed with the tissue section. In some embodiments, the assignment of a first or second attribute to a respective representation of a capture spot is performed using an algorithm, function and/or a script (e.g., Python). In some such embodiments the assignment is performed using the classification module 1120. In some embodiments, the algorithm returns a tissue position construct (e.g., a matrix, array, list or vector) comprising spatial coordinates as integers in row and column form, and barcode sequences for barcoded capture spots as values. In some embodiments, a tissue position construct is generated based on a plurality of parameters for an obtained image, including but not limited to a list of tissue positions, a list of barcoded capture spots, a list of the coordinates of the centers of each respective barcoded capture spot, one or more scaling factors for the obtained image (e.g., 0.0-1.0), one or more image masks generated by the heuristic classifiers and/or image segmentation algorithm, the diameter of a respective capture spot (e.g., in pixels), a data frame with row and column coordinates for the subset of capture spots overlayed with tissue, and/or a matrix comprising barcode sequences. In some such embodiments, the function for generating the tissue position construct determines which capture spots overlap the tissue section based on the spot positions and the tissue mask, where the overlap is determined as the fraction of capture spot pixels that overlap the mask. In some such embodiments, the calculation uses the radius of the capture spots and the scaling factor of the obtained image to estimate the overlap. In some embodiments, the function for generating the tissue position construct further returns an output including but not limited to a list of barcode sequences overlapping the tissue section, a set of scaled capture spot coordinates overlapping tissue, and/or a set of scaled capture spot coordinates corresponding to background.

In some embodiments, the plurality of capture spots 1202 are located directly below the tissue overlay image, while in some alternative embodiments, the plurality of capture spots 1202 are provided on a substrate that is different from the substrate 904 on which the tissue section overlay is imaged. In some embodiments, the tissue section is overlayed directly onto the capture spots on a substrate, either prior to or after the imaging, and the association of the capture spots with the one or more analytes from the tissue occurs through direct contact of the tissue with the capture spots. In some embodiments, the tissue section is not overlayed directly onto the capture spots and the association of the capture spots with the one or more analytes from the tissue occurs through transfer of analytes from the tissue to the capture spots using a porous membrane or transfer membrane.

Referring to block 1038, in some embodiments, a capture spot 1202 in the set of capture spots comprises a capture domain. Referring to block 1040, in some embodiments, a capture spot 1202 in the set of capture spots comprises a cleavage domain. Referring to block 1042, in some embodiments, each capture spot in the set of spots is attached directly or attached indirectly to the substrate.

Referring to block 1044, in some embodiments, the one or more analytes comprise five or more analytes.

Referring to block 1046, in some embodiments, the corresponding spatial barcode encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

Referring to block 1048, in some embodiments, each respective capture spot 1202 includes 1000 or more probes. Referring to block 1050, each probe in the respective capture spot includes a poly-A sequence or a poly-T sequence and the corresponding spatial barcode that characterizes the respective capture spot. Referring to blocks 1052 and 1054, in some embodiments, each probe in the respective capture spot includes the same spatial barcode or a different spatial barcode from the plurality of spatial barcodes.

Referring to block 1056, in some embodiments, the one or more analytes is a plurality of analytes. A respective capture spot 1202 in the set of capture spots includes a plurality of probes. Each probe in the plurality of probes includes a capture domain that is characterized by a capture domain type in a plurality of capture domain types. Each respective capture domain type in the plurality of capture domain types is configured to bind to a different analyte in the plurality of analytes.

Thus, in some such embodiments, each capture domain type corresponds to a specific analyte (e.g., a specific oligonucleotide or binding moiety for a specific gene). In some embodiments, each capture domain type in the plurality of capture domain types is configured to bind to the same analyte (e.g., specific binding complementarity to mRNA for a single gene) or to different analytes (e.g., specific binding complementarity to mRNA for a plurality of genes).

Referring to block 1058, in some embodiments, the plurality of capture domain types comprises between 5 and 15,000 capture domain types and the respective capture probe plurality includes at least five probes for each capture domain type in the plurality of capture domain types.

Referring to block 1060, in some embodiments, the one or more analytes is a plurality of analytes. A respective capture spot 1202 in the set of capture spots includes a plurality of probes, each probe in the plurality of probes including a capture domain that is characterized by a single capture domain type configured to bind to each analyte in the plurality of analytes in an unbiased manner. Thus, in some such embodiments, the capture domain comprises a non-specific capture moiety (e.g., an oligo-dT binding moiety).

Referring to block 1062, in some embodiments, each respective capture spot 1202 in the set of capture spots is contained within a 100 micron by 100 micron square on the substrate 904. Referring to block 1064, in some embodiments, a distance between a center of each respective spot 1202 to a neighboring capture spot 1202 in the set of capture spots on the substrate 904 is between 50 microns and 300 microns. In some embodiments, a distance between a center of each respective spot 1202 to a neighboring capture spot 1202 is between 100 microns and 200 microns.

Referring to block 1066, in some embodiments, a shape of each capture spot 1202 in the set of capture spots on the substrate is a closed-form shape. Referring to block 1068, in some embodiments, the closed-form shape is circular, elliptical, or an N-gon, where N is a value between 1 and 20. Referring to block 1070, in some embodiments, the closed-form shape is hexagonal. Referring to block 1072, in some such embodiments, the closed-form shape is circular and each capture spot in the set of capture spots has a diameter of 80 microns or less. In some embodiments, the closed-form shape is circular or hexagonal, and each capture spot in the set of capture spots has a diameter of between 30 and 200 microns, and/or a diameter of 100 microns. Referring to block 1074, in some embodiments, the closed-form shape is circular and each capture spot in the set of capture spots has a diameter of between 30 microns and 65 microns. In some embodiments, the closed-form shape is circular or hexagonal and each capture spot in the set of capture spots has a diameter of 60 microns. Referring to block 1076, in some embodiments, a distance between a center of each respective capture spot to a neighboring capture spot in the set of capture spots on the substrate is between 50 microns and 80 microns.

In some embodiments, the positions of a plurality of capture spots of an array are predetermined. In some embodiments, the positioned of a plurality of capture spots of an array are not predetermined. In some embodiments, the substrate comprises fiducial markers, and the position of the fiducial markers is predetermined such that they can be mapped to a spatial location. In some embodiments, a substrate comprises 500 of more capture spots. In some embodiments, a substrate comprises between 1000 and 5000 capture spots, where capture spots are arranged on the substrate hexagonally or in a grid.

Numerous alternative combinations of capture domain types, capture spot sizes, arrays, probes, spatial barcodes analytes, and/or other features of capture spots including but not limited to dimensions, designs, and modifications are also possible, and are discussed in detail at length above (e.g., in Section (II) General Spatial Array-Based Analytical Methodology; Subsections (b) Capture Probes, (c) Substrate, and (d) Arrays).

The present embodiments can be implemented as a computer program product that comprises a computer program mechanism embedded in a nontransitory computer readable storage medium. For example, the computer program product could contain the program modules shown in FIG. 11, and/or described in FIGS. 12A, 12B, 12C, 12D, 12E, and 12F. These program modules can be stored on a CD-ROM, DVD, magnetic disk storage product, USB key, or any other non-transitory computer readable data or program storage product.

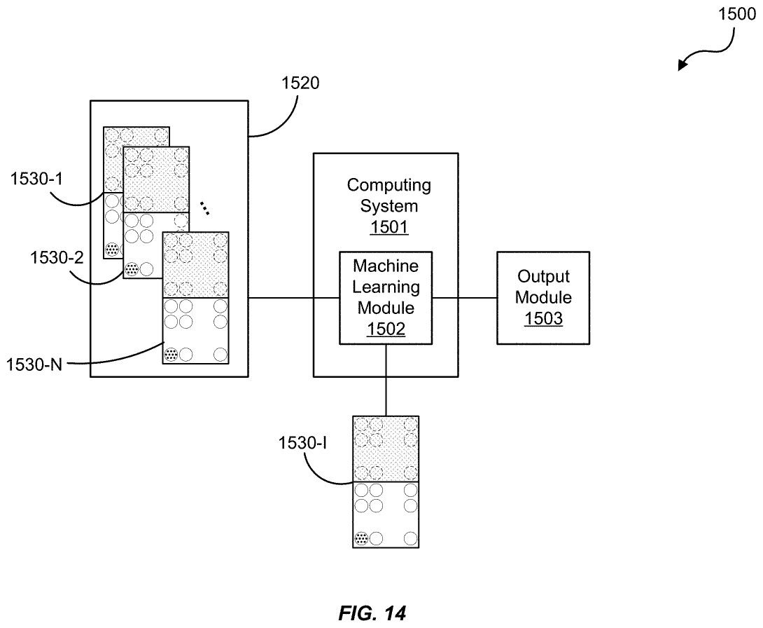

FIG. 14 is a block diagram of an exemplary system 1500 operable to identify and/or predict features, such as gene expression, protein expression, etc., in a biological sample. In this embodiment, the system 1500 is implemented with a computing system 1501 which may be representative of the system 1100 of FIG. 11. For example, the computing system 1501 may include one or more processors, storage devices (e.g., persistent and/or volatile storage devices including computer memory, solid-state drives, hard disk drives, etc.), network interfaces, graphics cards, etc. The computing system 1501 may be operable to implement a machine learning module 1502. In this regard, the machine learning module 1502 may be implemented as combination of computer hardware, software, and/or firmware configured with the computing system 1501, including graphics cards capable of parallel processing.

The computing system 1501 may be operable to process a plurality of datasets 1530-1-1530-N (where the reference “N” is an integer greater than “1” and not necessarily equal to any other “N” reference designated herein). Each dataset 1530 may include molecular measurement data of a biological sample (e.g., data pertaining to captured analytes of a biological sample) and image data of the biological sample that is registered to areas of the biological sample where the molecular measurement data is captured.

For example, the biological sample may interrogated using any of a variety of molecular measurement techniques at a plurality of capture areas, such as the capture area 801 of FIG. 8, shown and described above. The molecular measurement techniques may include barcodes that sample for mRNA target analytes. Alternatively or additionally, the molecular measurement techniques may employ random or degenerate N-mer captures for gDNA analysis, employ captures of an analyte using a capture agent 805, bind nucleic acid molecules 806 that can function in a CRISPR assay (e.g., CRISPR/Cas9), attach antibodies to molecules, use a poly-A capture technique that employs poly-dT oligos and spatial barcodes which hybridize to a poly-A tail of mRNA to capture gene expression data, capture protein expression data with a plurality of antibody probes, and the like. Any of these and/or other molecular measurement techniques may be employed at any or all of the capture areas of the biological sample.

An image of the biological sample may be obtained with fiducial markers, as shown and described above. The fiducial markers of the image may be used to align the image of the biological sample with the molecular measurement data at known locations. The image may comprise image data that includes pixel location, intensity, contrast, brightness, color (e.g., hue), grayscale, etc. for each pixel in the image. This image data may be linked to the known locations of the capture areas where the molecular measurement techniques interrogate the biological sample. Once generated, all or a portion of the datasets 1530-1-1530-N may be used to train the machine learning module 1502 to predict or otherwise identify analytes, such as gene expression and/or protein expression, in the dataset 1530-I of another biological sample.

Machine learning generally regards algorithms and statistical models that computer systems, such as the computing system 1501, use to perform a specific task without using explicit instructions, relying on patterns and inference instead. For example, machine learning algorithms may build a mathematical model based on sample data, known as “training data”, in order to make predictions or decisions without being explicitly programmed to perform the task. Thus, when a plurality of biological samples is obtained from similar tissue samples, a dataset 1530 from each biological sample may be generated to provide a data library 1520 that may be used to train the machine learning module 1502 of the computing system 1501. Typically, many datasets 1530 are used (e.g., hundreds, thousands, or more) because a larger number of datasets provides a better statistical model to predict features in another biological sample.

From there, the machine learning module 1502 may detect anomalies in the biological samples and/or identify various features (e.g., analytes) in the biological samples, such as gene and/or protein expression. In one embodiment, the machine learning module 1502 may operate on the data library 1520 to learn features in each of the datasets 1530 to determine whether a similar feature exists in a dataset 1530-I. For example, the machine learning module 1502 may be trained with each of the datasets 1530 of the data library 1520 to learn features in image data and/or learn features, such as gene expression, protein expression, etc., in the molecular measurement data. Thereafter, the machine learning module 1502 may identify similar features in the dataset 1530-I, and output those to an output module 1503. In some embodiments, the machine learning module 1502 may be operable to detect features of the biological sample 1530-I through the use of supervised learning.

In some embodiments, the training data may be, or include, simulated data. For example, the physics and biology regarding biological processes of disease tissue, healthy tissue, therapeutic responses and responders, the boundaries of tissue, etc. may be used as rules to generate data that can be formatted in a manner that would appear as actual data (e.g., with molecular measurement data registered to image data). Then, this simulated data can be used either alone or in conjunction with actual data to train the machine learning module 1502.

The machine learning module 1502 is not intended to be limited to a particular machine learning algorithm. Rather, the machine learning module 1502 may employ one or more of a variety of machine learning algorithms. Just a few examples of machine learning algorithms that may be implemented by the machine learning module 1502 include a supervised learning algorithm, a semi-supervised learning algorithm, an unsupervised learning algorithm, a regression analysis algorithm, a reinforcement learning algorithm, a self-learning algorithm, a feature learning algorithm, a sparse dictionary learning algorithm, an anomaly detection algorithm, a generative adversarial network algorithm, a transfer learning algorithm, and an association rules algorithm.

In some embodiments, the image data may be used to train the machine learning module 1502 to identify locations in a sample that may include variations in the amount of a material in the sample. For example, a portion of an imaged sample may include a higher intensity than other portions of the image. This may indicate that there is more of the target analyte (e.g., DNA) at that location. This relationship may then be used to train the machine learning module 1502 to identify analyte densities in other images.

Based on the foregoing, the computing system 1501 is any device, system, software, or combination thereof operable to implement a machine learning module 1502 and to train the machine learning module 1502 with a plurality of datasets 1530 of biological samples. From there, the computing system 1501 may process the dataset 1530-I of another biological sample through the trained machine learning module to learn various features of the biological sample. In this regard, the computing system 1501 may be programmed with software to transform the computing system 1501 into a special purpose computing system for analyzing data pertaining to biological samples.

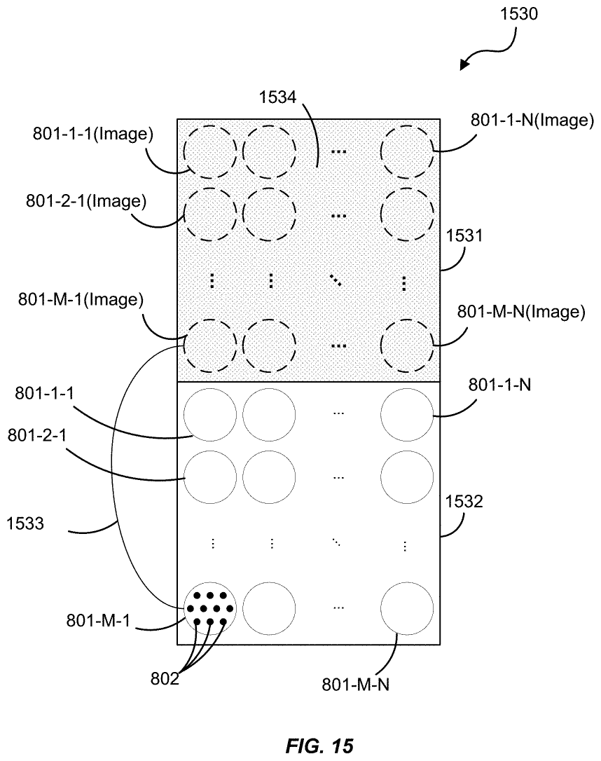

In FIG. 15, one exemplary dataset 1530 is illustrated. In this embodiment, the dataset 1530 comprises an image 1531 of a biological sample made up of an array of pixels 1534. The dataset 1530 also comprises data from an M×N array 1532 of capture areas 801 where the biological sample in interrogated (wherein the references “M” and “N” are integers greater than “1” and not necessarily equal to any other “M” and “N” reference is designated herein). Here, only the array of pixels 1534 is shown without colors or intensities to simplify the understanding of the registration process that links the capture areas 801 (e.g., and the molecular measurement data captured therefrom) to the pixels of the image.

To illustrate, the capture area 801-M−1 of the sample may comprise data from a plurality of capture points 802 where the molecular measurement data was obtained (e.g., via barcoding, antibodies, etc.). This capture area 801-M−1 is linked (1533) to a corresponding location 801-M−1 (Image) in the image 1531 of the biological sample, thereby registering the molecular measurement data to the pixel data of the image 1531. This registration generally involves mapping either the molecular measurement data of the capture points 802 to the image data, or vice versa, and establishing a common coordinate system for both sets of data such that co-analysis of molecular measures, such as gene expression and/or protein expression, and imaging measures can be performed.

In some embodiments, this registration involves aligning the molecular data coordinate system to the image data coordinate system. Generally, the “resolution” of the molecular data is much lower than the resolution of the image data. For example, the capture area 801-M−1 may comprise data pertaining to 50 or more barcoded analytes. However, the image data in the capture area 801-M−1 may be on the order of thousands of pixels (e.g., depending on the resolution of the imaging device). Accordingly, the computing system 1501 may either summarize the image data at the lower resolution of the molecular data or interpolate the molecular data to the resolution of the image data. And, with the capture points 802 linked to the pixel locations of the image, various molecular features can be visualized to identify, for example, gene expression, protein expression, diseased tissue, healthy tissue, boundaries of diseased tissue, boundaries of healthy tissue, etc.).

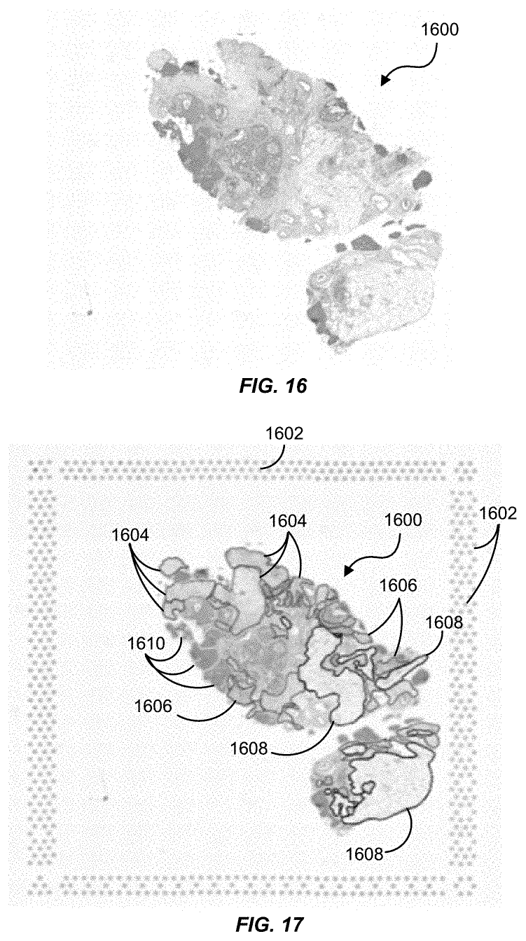

In some embodiments, the image data may be processed to identify or otherwise extract certain features from the image. To illustrate, a tissue sample is shown in FIG. 16 that was obtained from a Ductal Carcinoma In Situ (DCIS) pathology analysis. The tissue sample may be situated on a substrate that includes a plurality of fiducial markers 1602 that are used to register the molecular measurement data to the image data, as shown in FIG. 17. From there, the tissue sample 1600 may be imaged and processed to identify certain features. For example, the computing system 1501 may be configured (e.g., programmed) with software, such as Matlab by the MathWorks Corporation and/or Wolfram Mathematica, that may be used to extract features from the image. One example includes applying various filters, such as Gabor filters, to the image 1600.

Gabor filters are mathematical constructs that operate on neighborhoods of pixels and assign response values to those pixel neighborhoods. For example, Gabor filters have both an associated scale (i.e., size) and angle, and are configured in “banks” that assign a mathematical “fingerprint” to a “texture” in the image. In this regard, the image of the tissue sample 1600 may be convolved with each filter so as to replace the color at each pixel in the image with a vector of numbers that represents the response to each of those Gabor filters (e.g., a feature vector). Thus, by convolving the image with the Gabor filters, the computing system 1500 is operable to generate a numerical texture fingerprint centered at each pixel location in the image 1600. And, coupled with the molecular measurement data obtained at known locations in the image 1600, certain features of the tissue sample 1600, such as gene expression, protein expression, diseased tissue, etc., can be used to annotate the vector for the image. Once each dataset 1530 has been processed in similar fashion, the vectors of the datasets 1530-1-1530-N may be input to the machine learning module 1502 of the computing system 1501 as training data to predict various features, such as gene expression, protein expression, diseased tissue, etc., in a subsequent image (e.g., the image data of the dataset 1530-I of FIG. 14). Of course, it should be noted that the embodiments herein are not intended to be limited to just image processing via the use of Gabor filters, as image processing may be performed in a variety of ways as a matter of design choice.

In some embodiments, image processing techniques may illustrate various features in the image, such as regions in the tissue sample 1600. For example, in FIG. 17, the imaged tissue sample 1600 is illustrated with the regions 1604, 1606, 1608, and 1610 that correspond to different features in the tissue sample 1600. The image processing may better define these regions. And, coupled with the molecular measurement data at these regions, a pathologist may be able to determine what the regions in the tissue sample 1600 represent. In this example, the regions 1604 represent fibrous tissue cells, the regions 1606 represent immune cells, the regions 1608 represent fat cells, and the regions 1610 represent DCIS cancer cells.

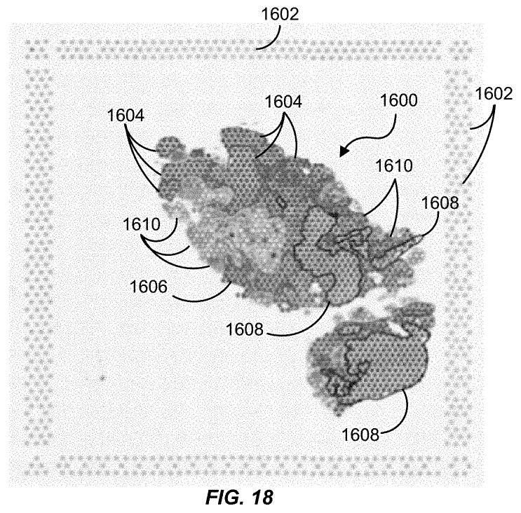

Now turning to FIG. 18, the molecular measurement data is linked to the image data via the fiducial markers 1602 in the image. For example, the capture areas where the molecular measurements were made on the tissue 1600 are registered to (e.g., aligned with) specific locations in the image of the tissue sample 1600. As can be seen in this example, the image of the tissue sample 1600 appears to be overlaid with a plurality of dots or “quasi pixels”. These quasi pixels may represent the capture areas where the molecular measurements are made and appear as a lower resolution form of the image of the tissue sample since the capture area resolution of the tissue sample is much lower resolution than the image data (i.e., pixel data) of the tissue sample. More specifically in this example, these quasi pixels may represent “counts” of gene expression that can be represented by integer values. The numbers of molecules indicate mRNA that was expressed for an ERBB2 gene. And, the regions 1610 illustrate increased counts for the ERBB2 gene (e.g., in the 20 to 50 range), thereby allowing a pathologist or other suitable professional to identify the region 1610 as cancerous.

With the regions 1604, 1606, 1608, and 1610 identified, the pixels in the image of the tissue sample can be labeled for training the machine learning module 1501 of FIG. 14. Again, in this example, there are four different regions identified: fibrous tissue cells (region 1604); immune cells (region 1606); fat cells (region 1608); and DCIS cancer cells (region 1610). Thus, the pixels in each of the regions may be annotated with labels identifying those regions. For example, assume that the pixel data of the image can be represented by four colors, one for each of the four identified regions. Then, the regions can self-annotate by the colors themselves and can be represented by two bits of data (i.e., 00 for region 1604, 01 for region 1606, 10 for region 1608, and 11 for region 1610).

And, as such can be done with each image of a dataset 1530, the machine learning module 1502 can be trained with the image data in the datasets 1530-1-1530-N to identify similar regions in subsequent datasets, such as the dataset 1530-I. That is, the machine learning module 1502, through supervised learning, may learn the labeled features of the images from the datasets 1530-1-1530-N and predict or otherwise identify similar features in the image data of the dataset 1530-I. Of course, the pixels are typically represented by many more bits and annotation can be represented by additional bits.

Alternatively or additionally, the molecular measurement data may be used to predict an image of a tissue sample. For example, the molecular measurement data may be obtained at known locations of the tissue sample 1600 (e.g., via capture probes capturing molecular measurement data at the capture points 802 in the capture areas 801). More specifically, the fiducial alignment process disclosed herein may also be employed by the molecular measurement techniques such that the captured molecular measurement data at the capture areas 801 may align with the fiducial markers of an image, such as the fiducial markers 1602 in FIG. 17. Thus, the location of the captured molecular measurement data is known.

To illustrate in one example, assume that the captured molecular measurement data can be represented as an M×N matrix of “x” (again, where “M” and “N” are integers greater than “1” and not necessarily equal to any other “M” or “N” references designated herein), where:

x=

| Bar- | Bar- | Bar- | Bar- | Bar- | Bar- | | Bar- | |
|---|---|---|---|---|---|---|---|---|
| code | code | code | code | code | code | | code | |
| 1 | 2 | 3 | 4 | 5 | 6 | . . . | M | |
| 0 | 1 | 60 | 0 | 0 | 0 | | 90 | Gene 1 |
| 0 | 0 | 0 | 0 | 0 | 0 | | 0 | Gene 2 |
| | | | | | | | | . . . |
| 0 | 90 | 20 | 4000 | 20 | 8 | | 0 | Gene N |

This matrix illustrates counts of molecules that were observed via barcodes, where the various barcodes employed form the columns of the matrix and the molecules of the genes detected form the rows of the matrix. As mentioned, the number of barcodes is typically much smaller than the number of image positions, so the molecular measurement data is sparser in space than the image data (e.g., lower resolution). The molecular measurement data is also sparse in terms of counts. That is, not all genes are observed at all locations and many genes may not be observed at all.

Then, for each image coordinate (x, y) in a pathology image, such as that of FIG. 17, there is a value representing a pathologist's interpretation of the type of tissue that is present (e.g., fibrous tissue cells of region 1604, immune cells of region 1606, fat cells of region 1608, and DCIS cancer cells of region 1610). This may represent a categorical choice at each pixel. And, in some instances, this could be a vector of probabilities demonstrating confidence between multiple choices. Thus, when the locations of the captured molecular measurement data are known in a relatively large number of datasets 1530, the captured molecular measurement data (e.g., along with the image data) can be used as training data for the machine learning module 1502 so as to predict an image of a subsequent dataset, such as the molecular measurement data of the dataset 1530-I.

In one exemplary embodiment, gene expression data may be aligned to an image, as described herein, to convert a matrix of gene “x” to a matrix of gene “x” image position. Again, this data is generally sparse in terms of genes represented. Accordingly, the computing system 1501 may perform a dimensionality reduction (e.g., via principal component analysis, or “PCA”, and using the top “K” components). This may produce a matrix of “K” by “x” image positions, where the image positions are sparser than the number of pixels in the original image. Thus, the computing system 1501 may interpolate the captured molecular measurement data using an appropriate multidimensional interpolation, such as co-kriging. Cokriging is a geostatistical technique used for interpolation in mapping and image contouring. This may produce a matrix of the captured molecular measurement data that is the same size as the image where, for each (x, y) coordinate in the image, there is a K-dimensional vector that may be used to train the machine learning module 1502 (i.e., for each of the datasets 1530 in the data library 1520). Alternatively, training and inference of the machine learning module 1502 could be performed at image positions that have molecular measurement data.

In some embodiments, the machine learning module 1502 could be trained to label a dataset, such as the dataset 1530-1. For example, using any of the data shown and described above, the machine learning module 1502 may employ a random forest classifier that can be trained to label images, and/or label features, such as gene expression, protein expression, diseased tissue, etc., in a dataset 1530-1 using labeled results of image data, gene expression data, protein expression, pathologist annotations, and the like, from the datasets 1530-1-1530-N. However, any number of multi-category learning methods could be used. In this regard, the machine learning module 1502 may be trained using data triplets (e.g., image data, molecular measurement data, and pathologist annotations) from a relatively large number of datasets 1530. Once trained, the machine learning module 1502 may automatically label subsequent datasets by preprocessing molecular measurement data in the same way (e.g., via dimensionality reduction and/or interpolation). Then, the machine learning module 1502 can preprocess the image data in a subsequent dataset 1530-1 to extract the same or similar image measures from the image data of the dataset 1530-1 (e.g, via Gabor filter bank responses),

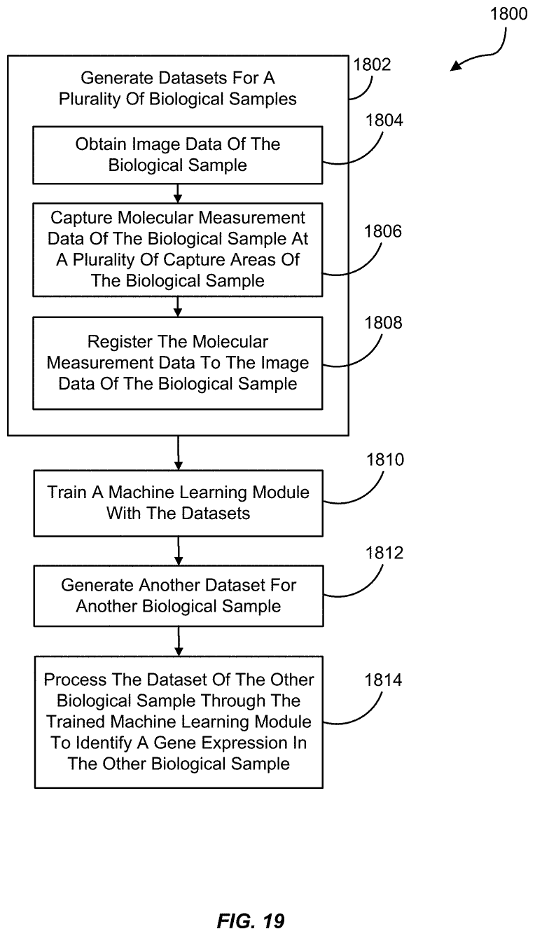

FIG. 19 is a flowchart of an exemplary process 1800 that may be performed by, or in conjunction with, the computing system 1501 of FIG. 14. In this embodiment, datasets for a plurality of biological samples are generated, in the process element 1802 (e.g., the datasets 1530-1-1530-N). For example, a tissue sample may be placed on a substrate comprising a plurality of fiducial markers and then imaged (e.g., with a high-resolution camera) to obtain image data of the tissue sample, in the process element 1804. And, molecular measurement data of the tissue sample may be captured at a plurality of capture areas of the biological sample, in the process element 1806. This capturing may include, at specific capture areas of the biological sample, barcoding analytes of the biological sample, tagging the sample with antibodies, and the like, as shown and described above. Then, the computing system 1501 may register the molecular measurement data of the capture areas to the image data of the biological sample, in the process element 1808, using the fiducial markers.

From there, the computing system 1501 may use the datasets to train the machine learning module 1502 (e.g., via a supervised learning process or a feature learning process), in the process element 1810. For example, the computing system 1501 may perform image processing on the image data to extract various features from a biological sample and convert these features into a vector of numbers. And, with the molecular measurement data of the biological sample being registered to the known locations of the image data, each vector may be annotated with certain features of the biological sample, such as gene expression, protein expression, immune cells, diseased tissue, etc. The computing system 1501 may then use these annotated vectors to train the machine learning module 1502. Alternatively, the image data itself may be annotated with the features to train the machine learning module 1502. In some embodiments, the molecular measurement data of the biological samples may be used to train the machine learning module 1502 (e.g., to predict an image of a subsequent biological sample).

Whatever the case, when another biological sample is to be analyzed, another dataset for the biological sample may be generated, in the process element 1812. Then, the machine learning module 1502 may process the dataset of the other biological sample to identify features in the other biological sample, in the process element 1804.

In some embodiments, the machine learning module 1502 may be operable to predict a likelihood of disease in a biological sample based on an identified gene expression of the biological sample, determine a change in a gene expression profile pertaining to a biological sample based on the identified gene expression of the biological sample, determine a change in morphology pertaining to a biological sample based on the identified gene expression of the biological sample, determine a change in protein expression to a biological sample based on the identified gene expression of the biological sample, and/or determine tissue susceptibility to therapeutics in a biological sample based on the identified gene expression of the biological sample.

For example, certain identified genes impact morphology. Thus, one or more clinical biomarkers on an RNA level may be identified (e.g., by the machine learning module 1502 being trained on multimodal data) that would lead to a change in the morphology on a level not previously detectable by an experienced pathologist, thus allowing better actionable decision making by the pathologist. Some examples of biomarkers include HER2, ER, PGR, PD1/PDL1. Of course, the embodiments herein are not intended to be limited to such biomarkers. Rather, more complex biomarkers could be identified from scores that are output of the trained machine learning module 1502.

Examples of therapeutic responses being linked to gene expression may also be identified, such as where a decrease in gene expression may reflect better outcomes (e.g., responsiveness). For example, a higher PD1/PDL1 protein expression (e.g., significant biomarkers currently present in a large number of clinical studies) may be directly correlated with a response to PD1/PDL1 inhibitors for treatment of several cancers.

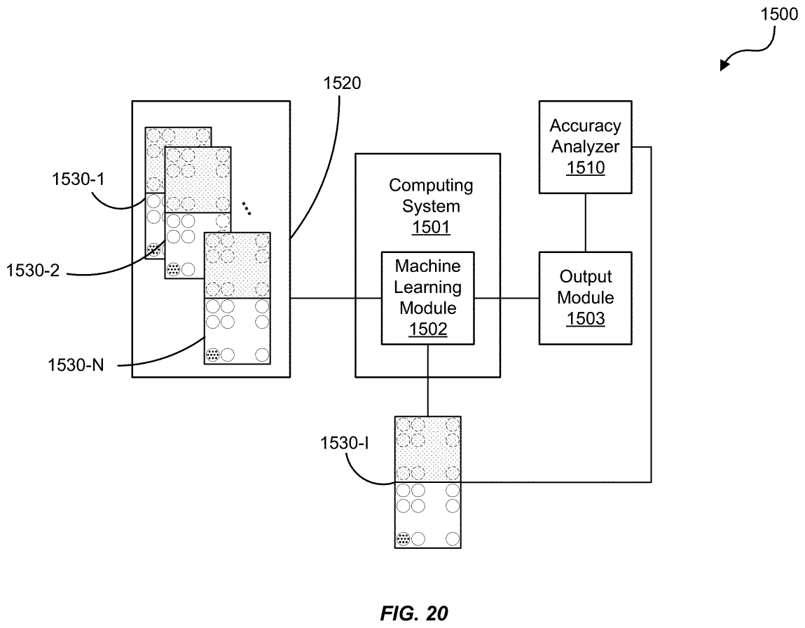

FIG. 20 is a block diagram of the system 1500 configured with an accuracy analyzer 1510 that may be operable to determine a level of accuracy for the machine learning module 1502. For example, empirical data may be obtained for another biological sample which is represented by the dataset 1530-I. Then, data from the dataset 1530-I (e.g., molecular measurement data, pathology annotations, and/or image data) may be input to the trained machine learning module 1502 such that the machine learning module 1502 can learn or otherwise predict features in the other biological sample. These learned features may then be output to the output module 1503 and compared to the empirical data of the other biological sample by the accuracy analyzer to determine a level of accuracy for the machine learning module 1502.

For example, suppose the molecular measurement data of the dataset 1530-I was found, from the previous example, to be x=

| Bar- | Bar- | Bar- | Bar- | Bar- | Bar- | | Bar- | |
|---|---|---|---|---|---|---|---|---|
| code | code | code | code | code | code | | code | |
| 1 | 2 | 3 | 4 | 5 | 6 | . . . | M | |
| 0 | 1 | 60 | 0 | 0 | 0 | | 90 | Gene 1 |
| 0 | 0 | 0 | 0 | 0 | 0 | | 0 | Gene 2 |
| | | | | | | | | . . . |
| 0 | 90 | 20 | 4000 | 20 | 8 | | 0 | Gene N |

Now suppose that the machine learning module 1502 predicted in the dataset 1530-I (e.g., based in part on the training from the image data of the datasets 1530-1-1530-N) x′=

| Bar- | Bar- | Bar- | Bar- | Bar- | Bar- | | Bar- | |
|---|---|---|---|---|---|---|---|---|
| code | code | code | code | code | code | | code | |
| 1 | 2 | 3 | 4 | 5 | 6 | . . . | M | |
| 0 | 1 | 50 | 0 | 0 | 0 | | 90 | Gene 1 |
| 0 | 0 | 0 | 0 | 0 | 0 | | 0 | Gene 2 |
| | | | | | | | | . . . |
| 0 | 90 | 20 | 4050 | 20 | 8 | | 0 | Gene N |

The accuracy analyzer 1510 could then compare the empirical molecular measurement data x of the dataset 1530-I to the predicted molecular measurement data x′ of the dataset 1530-I to determine some percentage of accuracy for the machine learning module 1502.

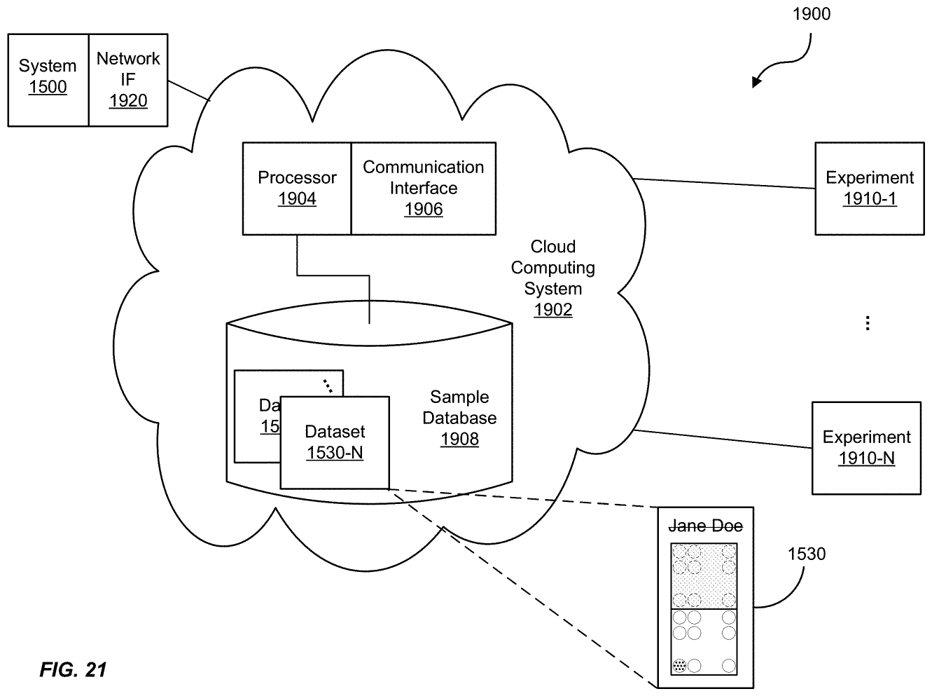

FIG. 21 is a block diagram of the system 1500 of FIG. 14 being implemented as a network-based system 1900. For example, the system 1500 may store the datasets 1530-1-1530-N in a cloud computing system 1902. In this regard, the system 1500 may include a network interface 1920 that is operable to communicate the datasets 1530 to the cloud computing system 1902. In some embodiments, the datasets 1530 are anonymized as data structures in a sample database 1908. For example, each dataset 1530 may represent image data and molecular measurement data of a biological sample of an individual. The personally identifiable information (PII), such as name, address, etc. of the individual is removed. In some embodiments, the age, ethnicity, geographical region, and the like are retained so as to categorize the biological samples accordingly. For example, the machine learning module 1502 may be trained with datasets of similar tissue samples of individuals of a similar age. Then, features of a subsequent biological sample (e.g., the dataset 1530-I) can be predicted for an individual of that age.

In some embodiments, the cloud computing system 1902 includes a processor 1904 that is operable to implement the machine learning module 1502. In this regard, external experiments 1910-1-1910-N may be performed on other biological samples. For example, molecular measurement data and/or image data of the datasets 1530 may be retrieved by the experiments 1910-1-1910-N. Then, this data may be processed through the trained machine learning module 1502 configured with the experiments 1910-1-1910-N to predict features in the molecular measurement data and/or the image data obtained in the experiments 1910 in a manner similar to that shown and described herein. The results of these experiments 1910 may also be uploaded to the cloud computing system 1902 and stored with the sample database 1908 such that the machine learning module 1502 can be retrained to improve the accuracy of the machine learning module 1502. Alternatively, the external experiments 1910 may access the trained machine learning module 1502 configured with the system 1500. In some embodiments, the sample database 1908 is secured such that only authorized experiments 1910 may be granted access to the machine learning module 1502.

Modifications and variations of the embodiments herein can be made without departing from its spirit and scope, as will be apparent to those skilled in the art. The specific embodiments described herein are offered by way of example only. The embodiments were chosen and described in order to explain certain principles and practical applications of the embodiments to thereby enable others skilled in the art to best utilize the various embodiments with various modifications that may be suited to the particular use contemplated.

What is claimed is:

1. A computer implemented method, comprising:
generating datasets for a plurality of biological samples, wherein the dataset of each biological sample comprises:
image data of the biological sample; and
molecular measurement data of the biological sample captured at a plurality of capture areas of the biological sample, wherein the capture areas of the biological sample are registered to corresponding locations in the image data of the biological sample;
training a machine learning module with the datasets;
generating another dataset for another biological sample; and
processing the other dataset of the other biological sample through the trained machine learning module to predict features in the other biological sample.

2. The method of claim 1, further comprising:
selecting the plurality of biological samples from a plurality of tissue types.

3. The method of claim 1, further comprising:
selecting the plurality of biological samples from a single tissue type.

4. The method of claim 1, wherein generating the datasets further comprises:
capturing the molecular measurement data with at least one type of antibody at the capture areas of the biological samples.

5. The method of claim 1, wherein generating the datasets further comprises:
barcoding analytes at the capture areas of the biological samples to capture the molecular measurement data.

6. The method of claim 1, wherein generating the datasets further comprises:
capturing the molecular measurement data via a poly-A capture technique using poly-dT oligos and spatial barcodes which hybridize to a poly-A tail of mRNA to capture gene expression data.

7. The method of claim 1, wherein:
the molecular measurement data comprises gene expression data and protein expression data; and
generating the datasets further comprises:
capturing the gene expression data via poly-dT oligos and spatial barcodes; and
capturing the protein expression data with a plurality of antibody probes.

8. The method of claim 1, wherein generating the datasets further comprises:
obtaining an image of each of the biological samples to generate the image data, wherein each of the biological samples is overlaid on a substrate that includes one or more fiducial markers used to register the capture areas to the image.

9. The method of claim 8, further comprising:
filtering the image data of each biological sample to extract feature data from the image data.

10. The method of claim 1, wherein:
the machine learning module comprises at least one of a supervised learning module, a semi-supervised learning module, an unsupervised learning module, a regression analysis module, a reinforcement learning module, a self-learning module, a feature learning module, a sparse dictionary learning module, an anomaly detection module, a generative adversarial network, or an association rules module.

11. The method of claim 1, further comprising:
comparing the predicted features in the other biological sample to empirical data of the other biological sample to determine a level of accuracy of the machine learning module.

12. The method of claim 1, further comprising at least one of:
predicting a likelihood of disease in the other biological sample based on the predicted features of the other biological sample.
determining a change in a gene expression profile pertaining to the other biological sample based on the predicted features of the other biological sample;
determining a change in morphology pertaining to the other biological sample based on the predicted features of the other biological sample;
determining a change in protein expression to the other biological sample based on the predicted features of the other biological sample; or
determining tissue susceptibility to therapeutics in the other biological sample based on the predicted features of the other biological sample.

13. The method of claim 1, further comprising:
storing each dataset in a database;
securing the database; and
granting access to the secured database through a communication interface for external experimentation.

14. The method of claim 13, further comprising:
receiving results of the external experimentation through the communication interface; and
storing the results in the secured database to increase a number of the datasets for subsequent training of the machine learning module.

15. The method of claim 1, wherein:
training the machine learning module comprises training the machine learning module with the image data of the generated datasets and the molecular measurement data of the generated datasets; and
the method further comprises processing image data of the other dataset of the other biological sample through the trained machine learning module to predict molecular measurement data in the other biological sample.

16. The method of claim 1, wherein:
training the machine learning module comprises training the machine learning module with the image data of the generated datasets and the molecular measurement data of the generated datasets; and
the method further comprises processing molecular measurement data of the other dataset of the other biological sample through the trained machine learning module to predict an image in the other biological sample.

17. The method of claim 1, wherein:
training the machine learning module comprises training the machine learning module with the image data of the generated datasets, the molecular measurement data of the generated datasets, and at least one pathology annotation in each of the biological samples; and
the method further comprises processing image data and molecular measurement data of the other dataset of the other biological sample through the trained machine learning module to predict a disease state in the other biological sample.

18. A non-transitory computer readable medium comprising instructions that, when directed by a processor, direct the processor to:
generate datasets for a plurality of biological samples, wherein the dataset of each biological sample comprises:
image data of the biological sample; and
molecular measurement data of the biological sample captured at a plurality of capture areas of the biological sample, wherein the capture areas of the biological sample are registered to corresponding locations in the image data of the biological sample;
train a machine learning module with the datasets;
generate another dataset for another biological sample; and
process the other dataset of the other biological sample through the trained machine learning module to predict features in the other biological sample.

19. The computer readable medium of claim 18, wherein:
the plurality of biological samples are selected from a plurality of tissue types.

20. The computer readable medium of claim 18, wherein:
the plurality of biological samples are selected from a single tissue type.

21. The computer readable medium of claim 18, wherein:
the molecular measurement data is captured with at least one type of antibody at the capture areas of the biological samples.

22. The computer readable medium of claim 18, wherein:
analytes are barcoded at the capture areas of the biological samples to capture the molecular measurement data.

23. The computer readable medium of claim 18, wherein:
the molecular measurement data comprises gene expression data captured via a poly-A capture technique using poly-dT oligos and spatial barcodes which hybridize to a poly-A tail of mRNA.

24. The computer readable medium of claim 18, wherein:
the molecular measurement data comprises gene expression data and protein expression data;
the gene expression data is captured via poly-dT oligos and spatial barcodes; and
the protein expression data is captured with a plurality of antibody probes.

25. The computer readable medium of claim 18, wherein:
the image data of the datasets is generated from images of the biological samples, where each of the biological samples is overlaid on a substrate that includes one or more fiducial markers used to register the capture areas to the image.

26. The computer readable medium of claim 25, further comprising instructions that direct the processor to:
filter the image data of each biological sample to extract feature data from the image data.

27. The computer readable medium of claim 18, wherein:
the machine learning module comprises at least one of a supervised learning module, a semi-supervised learning module, an unsupervised learning module, a regression analysis module, a reinforcement learning module, a self-learning module, a feature learning module, a sparse dictionary learning module, an anomaly detection module, a generative adversarial network, or an association rules module.

28. The computer readable medium of claim 18, further comprising instructions that direct the processor to:
compare the predicted features in the other biological sample to empirical data of the other biological sample to determine a level of accuracy of the machine learning module.

29. The computer readable medium of claim 18, further comprising instructions that direct the processor to, at least one of:
predict a likelihood of disease in the other biological sample based on the predicted features of the other biological sample.
determine a change in a gene expression profile pertaining to the other biological sample based on the predicted features of the other biological sample;
determine a change in morphology pertaining to the other biological sample based on the predicted features of the other biological sample;
determine a change in protein expression to the other biological sample based on the predicted features of the other biological sample; or
determine tissue susceptibility to therapeutics in the other biological sample based on the predicted features of the other biological sample.

30. The computer readable medium of claim 18, further comprising instructions that direct the processor to:
store each dataset in a database;
secure the database; and
grant access to the secured database through a communication interface for external experimentation.

31. The computer readable medium of claim 30, further comprising instructions that direct the processor to:
receive results of the external experimentation through the communication interface; and
store the results in the secured database to increase a number of the datasets for subsequent training of the machine learning module.

32. The computer readable medium of claim 18, further comprising instructions that direct the processor to:
train the machine learning module with the image data of the generated datasets and the molecular measurement data of the generated datasets; and
process image data of the other dataset of the other biological sample through the trained machine learning module to predict molecular measurement data in the other biological sample.

33. The computer readable medium of claim 18, further comprising instructions that direct the processor to:
train the machine learning module with the image data of the generated datasets and the molecular measurement data of the generated datasets; and
process molecular measurement data of the other dataset of the other biological sample through the trained machine learning module to predict an image in the other biological sample.

34. The computer readable medium of claim 18, further comprising instructions that direct the processor to:
train the machine learning module with the image data of the generated datasets, the molecular measurement data of the generated datasets, and at least one pathology annotation in each of the biological samples; and
process image data and molecular measurement data of the other dataset of the other biological sample through the trained machine learning module to predict a disease state in the other biological sample.

35. A system, comprising:
a storage element operable to store a plurality of a datasets generated from a corresponding plurality of biological samples, wherein the dataset of each biological sample comprises:
image data of the biological sample; and
molecular measurement data of the biological sample captured at a plurality of capture areas of the biological sample, wherein the capture areas of the biological sample are registered to corresponding locations in the image data of the biological sample; and
a processor operable to implement a machine learning module, to train the machine learning module with the datasets, to process another dataset of another biological sample through the trained machine learning module, and to predict features in the other biological sample based on said processing.

36. The system of claim 35, wherein:
the plurality of biological samples are selected from a plurality of tissue types.

37. The system of claim 35, wherein:
the plurality of biological samples are selected from a single tissue type.

38. The system of claim 35, wherein:
the molecular measurement data is captured with at least one type of antibody at the capture areas of the biological samples.

39. The system of claim 35, wherein:
analytes are barcoded at the capture areas of the biological samples to capture the molecular measurement data.

40. The system of claim 35, wherein:
the molecular measurement data comprises gene expression data captured via a poly-A capture technique using poly-dT oligos and spatial barcodes which hybridize to a poly-A tail of mRNA.

41. The system of claim 35, wherein:
the molecular measurement data comprises gene expression data and protein expression data;
the gene expression data is captured via poly-dT oligos and spatial barcodes; and
the protein expression data is captured with a plurality of antibody probes.

42. The system of claim 35, wherein:
the image data of the datasets is generated from images of the biological samples, where each of the biological samples is overlaid on a substrate that includes one or more fiducial markers used to register the capture areas to the image.

43. The system of claim 43, wherein the processor is further operable to:
filter the image data of each biological sample to extract feature data from the image data.

44. The system of claim 35, wherein:
the machine learning module comprises at least one of a supervised learning module, a semi-supervised learning module, an unsupervised learning module, a regression analysis module, a reinforcement learning module, a self-learning module, a feature learning module, a sparse dictionary learning module, an anomaly detection module, a generative adversarial network, or an association rules module.

45. The system of claim 35, wherein the processor is further operable to:
compare the predicted features in the other biological sample to empirical data of the other biological sample to determine a level of accuracy of the machine learning module.

46. The system of claim 35, wherein the processor is further operable to, at least one of:
predict a likelihood of disease in the other biological sample based on the predicted features of the other biological sample.
determine a change in a gene expression profile pertaining to the other biological sample based on the predicted features of the other biological sample;
determine a change in morphology pertaining to the other biological sample based on the predicted features of the other biological sample;
determine a change in protein expression to the other biological sample based on the predicted features of the other biological sample; or
determine tissue susceptibility to therapeutics in the other biological sample based on the predicted features of the other biological sample.

47. The system of claim 35, wherein the processor is further operable to:
store each dataset in a database configured with the storage element;
secure the database; and
grant access to the secured database through a communication interface for external experimentation.

48. The system of claim 47, wherein the processor is further operable to:
receive results of the external experimentation through the communication interface; and
store the results in the secured database to increase a number of the datasets for subsequent training of the machine learning module.

49. The system of claim 35, wherein the processor is further operable to:
train the machine learning module with the image data of the generated datasets and the molecular measurement data of the generated datasets; and
process image data of the other dataset of the other biological sample through the trained machine learning module to predict molecular measurement data in the other biological sample.

50. The system of claim 35, wherein the processor is further operable to:
train the machine learning module with the image data of the generated datasets and the molecular measurement data of the generated datasets; and
process molecular measurement data of the other dataset of the other biological sample through the trained machine learning module to predict an image in the other biological sample.

51. The system of claim 35, wherein the processor is further operable to:
train the machine learning module with the image data of the generated datasets, the molecular measurement data of the generated datasets, and at least one pathology annotation in each of the biological samples; and
process image data and molecular measurement data of the other dataset of the other biological sample through the trained machine learning module to predict a disease state in the other biological sample.
